(19) United States
(12) Patent Application Publication
(10) Pub. No.: US 2023/0281444 A1
(43) Pub. Date: Sep. 7, 2023

(54) COMPUTATIONAL SYSTEM AND ALGORITHM FOR SELECTING NUTRITIONAL MICROORGANISMS BASED ON IN SILICO PROTEIN QUALITY DETERMINATION

(71) Applicant: Cella Farms Inc, San Ramon, CA (US)

(72) Inventors: Shane Brubaker, El Cerrito, CA (US); Monica Bhatia, San Ramon, CA (US); Keith McCall, København (DK); Baljit Singh Ghotra, San Ramon, CA (US)

(21) Appl. No.: 18/117,151

(22) Filed: Mar. 3, 2023

Related U.S. Application Data
(60) Provisional application No. 63/316,848, filed on Mar. 4, 2022.

(51) Int. Cl. G06N 3/08; G16B 35/00; G16B 40/20; G16B 20/00

(52) U.S. Cl. CPC G06N 3/08; G16B 35/00; G16B 40/20; G16B 20/00

(57) ABSTRACT

Provided are in silico methods for utilizing an algorithm and machine learning model to compute a protein nutritional quality score for an organism from the organism's genome and to select an organism as a source of protein based on a computed protein nutritional quality score.

### CROSS-REFERENCE TO RELATED APPLICATIONS

This application claims benefit of priority to U.S. Provisional Patent Application No. 63/316,848, filed Mar. 4, 2022, the disclosure of which is incorporated herein by reference in its entirety for all purposes.

### FIELD

The field of this disclosure is directed to the analysis of an organism's ability to be utilized as a dietary protein source. In certain aspects, the disclosure provides in silico methods to calculate a microorganism's protein nutritional quality score, by utilizing the microorganism's in silico genomic sequence. The disclosure further provides methods to increase the protein nutritional quality score of a composition with a low protein nutritional quality score by mixing with a composition with a higher protein nutritional quality score using the microorganism's protein nutritional quality score. The disclosure provides methods for the selection of microorganisms using an in silico method for use in the creation of high protein microbial food ingredients derived from fermentation.

### BACKGROUND

Dietary protein is an essential nutrient for human health and growth. The World Health Organization (WHO) recommends that dietary protein should contribute approximately 10 to 15% of energy intake when in energy balance and weight stable. Average daily protein intakes in various countries indicate that these recommendations are consistent with the amount of protein being consumed worldwide. Meals with an average of 20 to 30% of energy from protein are representative of high-protein diets when consumed in energy balance.

The human body cannot synthesize certain amino acids that are necessary for health and growth, and instead must obtain them from food. These amino acids, called “essential amino acids” (EAA), are Histidine (H), Isoleucine (I), Leucine (L), Lysine (K), Methionine (M), Phenylalanine (F), Threonine (T), Tryptophan (W), and Valine (V). Other categories of amino acids are called “non-essential amino acid” (NEAA) and “conditionally essential amino acid” (CEAA). NEAA includes Alanine (A), Arginine (R), Asparagine (N), Aspartic Acid (D), Glutamine (Q), Glutamic Acid (E), Glycine (G), Proline (P), and Serine (S). CEAA includes Cysteine (C) and Tyrosine (Y).

The minimum fractions of each essential amino acid that make up a healthy adult diet provide a metric against which dietary protein sources can be measured. For example, the fractions can be used to measure how well a dietary protein source meets the nutritional protein needs of humans, by targeting nutritionally balanced amino acid percentages. The World Health Organization (WHO) has developed a method for ranking the nutritional quality of a protein that compares the amino acid profile of the specific food protein against the amino acid requirements for human health. This method assigns a nutritional value to the protein called the “Protein Digestibility Corrected Amino Acid Score” (PDCAAS). An alternative to the PDCAAS standard is the “Digestible Indispensable Amino Acid Score” (DIAAS).

Determining the nutritional quality of a protein, either from PDCAAS or DIAAS, has been the most labor-intensive and costly element of a digestibility analysis. Much of the cost stems from using animals to provide digestibility data. Consequently, alternative methods to measure the nutritional quality of a protein can significantly reduce costs and further alleviate animal welfare concerns.

Dietary proteins that provide all the essential amino acids are referred to as “high quality” proteins. Animal foods such as meat, fish, poultry, eggs, and dairy products are generally regarded as high quality protein sources that provide a good balance of essential amino acids. The current methods to produce high quality protein, as found in animal food, generally involve a high carbon footprint, large land use, and significant consumption of water resources.

In contrast, foods that do not provide a good balance of essential amino acids are referred to as “low quality” proteins. Most fruits and vegetables are poor sources of protein and generally require less resources to produce compared to animal foods. While some plant foods, including beans, peas, lentils, nuts and grains (such as wheat) are better sources of protein, many of these foods are high in carbohydrates.

Soy, a vegetable protein manufactured from soybeans, is considered to be a high quality protein and the only plant protein that competes with meat.

As the world's populations shifts toward high-protein diets, more sustainable methods to produce high quality protein are needed. Moreover, developing new methods to identify high quality proteins is needed to sustain the shift toward high-protein diets. A gap exists to provide high quality protein in a way that does not consume significant resources.

### SUMMARY

The present disclosure solves the aforementioned problems by providing in silico methods to calculate an organism's protein nutritional quality and to select an organism to produce a protein ingredient based on the calculated protein nutritional quality.

In one aspect, provided herein is an in silico method for selecting an organism as a source of protein, the method comprising: (a) accessing a genomic library comprising genomic information; (b) creating an adjusted relative abundance proteomic library from the genomic library; (c) creating a functionally-characterized proteomic library from the adjusted relative abundance proteomic library; (d) supplying a computational algorithm with data from the functionally characterized proteomic library; (e) computing a protein nutritional quality score with the computational algorithm; and (f) selecting an organism as a source of protein from the genomic library, wherein the computational algorithm selects the organism based on its computed protein nutritional quality scores being above a desired threshold. In some cases, the computational algorithm comprises one or more computational algorithms. In some cases, one of the one or more computational algorithms is a machine learning algorithm. In some cases, the machine learning algorithm further computes protein digestibility factors. In some cases, the protein digestibility factor is an alpha helix/beta-sheet ratio. In some cases, the machine learning algorithm improves the accuracy of computing the digestibility factors. In some cases, the protein nutritional quality score is a protein expression estimation score, a protein molecular weight calculation score, and/or an amino acid analysis score. In some cases, the genomic library comprises a plurality of nucleotide sequences from a single organism. In some cases, the genomic library comprises a plurality of nucleotide sequences from a plurality of organisms. In some cases, the genomic library comprises at least one partial whole genome nucleotide sequence of an organism. In some cases, the genomic library comprises a plurality of partial whole genome nucleotide sequences of a plurality of organisms. In some cases, the genomic library comprises a plurality of complete whole genome nucleotide sequences of a plurality of organisms. In some cases, the genomic library is from a public genomic database. In some cases, the genomic library comprises a genomic sequence from a prokaryote. In some cases, the genomic library comprises a genomic sequence from a eukaryote. In some cases, the genomic library comprises a genomic sequence from an unknown organism. In some cases, the genomic library comprises a genomic sequence obtained from de novo sequencing. In some cases, the genomic library comprises a genomic sequence obtained from isolation sequencing. In some cases, creating the adjusted relative abundance proteomic library comprises direct translation of the genomic library. In some cases, creating the adjusted relative abundance proteomic library comprises direct translation of the microbial genomic library, and subsequent characterization of relative protein abundance. In some cases, creating the adjusted relative abundance proteomic library comprises calculation of a codon adaptation index parameter for each protein in the library. In some cases, creating the adjusted relative abundance proteomic library comprises calculation of a delta factor parameter comprising the Euclidean distance between each protein and the average ribosomal protein for each protein in the library. In some cases, creating the adjusted relative abundance proteomic library comprises mass spectrometry based shotgun proteomics. In some cases, creating the functionally characterized proteomic library comprises calculating one or more functional attributes of the library. In some cases, creating the functionally characterized proteomic library comprises calculating one or more functional attributes selected from the group consisting of: overall amino acid composition, essential amino acid composition, non-essential amino acid composition, most limiting amino acid, and estimated nitrogen content. In some cases, one or more modules of the computational algorithm may utilize a machine learning method selected from the group consisting of linear regression, kernel ridge regression, logistic regression, neural networks, support vector machines, decision trees, hidden Markov models, Bayesian networks, a Gram-Schmidt process, reinforcement-based learning, self-supervised learning, cluster-based learning, hierarchical clustering, language models, bi-directional Long-Short-Term-Memory and genetic algorithms. In some cases, the protein nutritional quality score is a Protein Digestibility Corrected Amino Acid Score (PDCAAS). In some cases, the protein nutritional quality score is a Digestible Indispensable Amino Acid Score (DIAAS). In some cases, the protein nutritional quality score is an in vitro Protein Digestibility Corrected Amino Acid Score (IVPDCAAS). In some cases, the protein nutritional quality score is an in vitro Digestible Indispensable Amino Acid Score (IVDIAAS). In some cases, the desired threshold of the IVDIAAS score is at least 100. In some cases, the desired threshold of the protein nutritional quality score is PDCAAS of at least 0.75. In some cases, the desired threshold of the protein nutritional quality score is DIAAS of at least 75. In some cases, the desired threshold of the protein nutritional quality score is IVPDCAAS of at least 0.75. In some cases, the desired threshold of the protein nutritional quality score is IVDIAAS of at least 75. In some cases, the protein nutritional quality score is a PDCAAS, DIAAS, IVPDCAAS, IVDIAAS, or any combination thereof. In some cases, the desired threshold of the protein nutritional quality score is PDCAAS, IVPDCAAS, DIAAS, IVDIAAS, or any combination thereof, each with a score of at least 0.75 and 75, respectively. In some cases, the protein nutritional quality score is a Euclidean distance metric. In some cases, the desired threshold of the Euclidean distance is less than 0.1 from a target amino acid distribution. In some cases, the target amino acid distribution is 60% essential amino acids and 40% non-essential amino acids. In some cases, the target amino acid distribution is 70% essential amino acids and 30% non-essential amino acids. In some cases, the target amino acid distribution is an amino acid distribution of proteins from milk. In some cases, the target amino acid distribution is an amino acid distribution of proteins from egg. In some cases, the target amino acid distribution is an amino acid distribution of proteins from beef. In some cases, the selected organism comprises a PDCAAS of at least 0.75. In some cases, the selected organism comprises a DIAAS of at least 75. In some cases, the selected organism comprises a Euclidean distance less than 0.1 from a target amino acid distribution. In some cases, the target amino acid distribution is 60% essential amino acids and 40% non-essential amino acids. In some cases, the target amino acid distribution is 70% essential amino acids and 30% non-essential amino acids. In some cases, the target amino acid distribution is an amino acid distribution of proteins from milk. In some cases, the target amino acid distribution is an amino acid distribution of proteins from egg. In some cases, the target amino acid distribution is an amino acid distribution of proteins from beef. In some cases, the selected organism is fermented to produce a protein ingredient. In some cases, the protein ingredient is used to improve the protein nutritional quality of a food product. In some cases, the food product is a human food product. In some cases, the human food product improves muscle health, brain health, pregnancy health, elderly health, epilepsy, diabetes, or cancer. In some cases, the food product is a companion animal food product. In some cases, the food product is a farm animal food product.

In another aspect, provided herein is an in silico method for determining an organism's protein nutritional quality from a genomic library, comprising: (a) accessing a genomic library; (b) creating an adjusted relative abundance proteomic library from the genomic library; (c) creating a functionally-characterized proteomic library from the adjusted relative abundance proteomic library; and (d) supplying a computational algorithm with data from the functionally characterized proteomic library, wherein the computational algorithm computes a protein nutritional quality score for an organism from the genomic library. In some cases, the computation algorithm is a machine learning algorithm. In some cases, the machine learning algorithm further computes protein digestibility factors. In some cases, the protein digestibility factor is an alpha helix/beta-sheet ratio. In some cases, the machine learning algorithm improves the accuracy of computing the digestibility factors. In some cases, the organism protein nutritional quality score is a protein expression estimation score, a protein molecular weight calculation score, and/or an amino acid analysis score. In some cases, the genomic library comprises a plurality of nucleotide sequences from a single microorganism. In some cases, the genomic library comprises a plurality of nucleotide sequences from a plurality of organisms. In some cases, the genomic library comprises at least one partial whole genome nucleotide sequence of an organism. In some cases, the genomic library comprises a plurality of partial whole genome nucleotide sequences of a plurality of organisms. In some cases, the genomic library comprises at least one complete whole genome nucleotide sequence of an organism. In some cases, the genomic library comprises a plurality of complete whole genome nucleotide sequences of a plurality of organisms. In some cases, the genomic library is from a public genomic database. In some cases, the genomic library comprises a genomic sequence from a prokaryote. In some cases, the genomic library comprises a genomic sequence from a eukaryote. In some cases, the eukaryote is a higher plant. In some cases, the genomic library comprises a genomic sequence from an unknown organism. In some cases, the genomic library comprises a genomic sequence obtained from de novo sequencing. In some cases, the genomic library comprises a genomic sequence obtained from isolation sequencing. In some cases, creating the adjusted relative abundance proteomic library comprises direct translation of the genomic library. In some cases, creating the adjusted relative abundance proteomic library comprises direct translation of the genomic library, and subsequent characterization of relative protein abundance. In some cases, creating the adjusted relative abundance proteomic library comprises calculation of a codon adaptation index parameter for each protein in the library. In some cases, creating the adjusted relative abundance proteomic library comprises calculation of a delta factor parameter comprising the Euclidean distance between each protein and the average ribosomal protein for each protein in the library. In some cases, creating the adjusted relative abundance proteomic library comprises mass spectrometry-based shotgun proteomics. In some cases, creating the functionally characterized proteomic library comprises calculating one or more functional attributes of the library. In some cases, creating the functionally characterized proteomic library comprises calculating one or more functional attributes selected from the group consisting of: overall amino acid composition, essential amino acid composition, non-essential amino acid composition, most limiting amino acid, and estimated nitrogen content. In some cases, one or more modules of the computational algorithm may utilize a machine learning method selected from the group consisting of linear regression, kernel ridge regression, logistic regression, neural networks, support vector machines, decision trees, hidden Markov models, Bayesian networks, a Gram-Schmidt process, reinforcement-based learning, self-supervised learning, cluster-based learning, hierarchical clustering, language models, bi-directional Long-Short-Term-Memory and genetic algorithms. In some cases, the organism protein nutritional quality score is a Protein Digestibility Corrected Amino Acid Score (PDCAAS). In some cases, the organism protein nutritional quality score is a Digestible Indispensable Amino Acid Score (DIAAS). In some cases, the organism protein nutritional quality score is an in vitro Protein Digestibility Corrected Amino Acid Score (IVPDCAAS). In some cases, the organism protein nutritional quality score is an in vitro Digestible Indispensable Amino Acid Score (IVDIAAS). In some cases, the IVDIAAS score is at least 100. In some cases, the organism protein nutritional quality score is PDCAAS of at least 0.75. In some cases, the organism protein nutritional quality score is DIAAS of at least 75. In some cases, the organism protein nutritional quality score is IVPDCAAS of at least 0.75. In some cases, the organism protein nutritional quality score is IVDIAAS of at least 75. In some cases, the organism protein nutritional quality score is a PDCAAS, DIAAS, IVPDCAAS, IVDIAAS, or any combination thereof. In some cases, the organism protein nutritional quality score is PDCAAS, IVPDCAAS, DIAAS, IVDIAAS, or any combination thereof, each with a score of at least 0.75 and 75, respectively. In some cases, the organism protein nutritional quality score is a Euclidean distance metric. In some cases, the Euclidean distance is less than 0.1 from a target amino acid distribution. In some cases, the target amino acid distribution is 60% essential amino acids and 40% non-essential amino acids. In some cases, the target amino acid distribution is 70% essential amino acids and 30% non-essential amino acids. In some cases, the target amino acid distribution is an amino acid distribution of proteins from milk. In some cases, the target amino acid distribution is an amino acid distribution of proteins from egg. In some cases, the target amino acid distribution is an amino acid distribution of proteins from beef.

In another aspect, provided herein is a processor-readable non-transitory medium storing code representing instructions to be executed by a processor, the code comprising code to cause the processor to: (a) access a microbial genomic library; (b) create an adjusted relative abundance microbial proteomic library from the microbial genomic library; (c) create a functionally characterized microbial proteomic library from the adjusted relative abundance microbial proteomic library; and (d) supply a computational algorithm with data from the functionally characterized microbial proteomic library, wherein the computational algorithm computes a protein nutritional quality score for a microorganism from the microbial genomic library.

In another aspect, provided herein is an in silico method for determining an organism's protein nutritional quality from a genomic library, comprising: (a) accessing a genomic library; (b) creating an adjusted relative abundance proteomic library from the genomic library; (c) creating a functionally characterized proteomic library from the adjusted relative abundance proteomic library; and (d) supplying a machine learning model with data from the functionally characterized proteomic library, wherein the machine learning model computes a protein nutritional quality score for an organism from the genomic library. In some cases, the organism is a prokaryote, and the genomic library is a prokaryotic genomic library. In some cases, the organism is a eukaryote, and the genomic library is a eukaryotic genomic library. In some cases, the organism is a yeast, and the genomic library is a yeast genomic library. In some cases, the organism is a plant, and the genomic library is a plant genomic library.

In another aspect, provided herein is a processor-readable non-transitory medium storing code representing instructions to be executed by a processor, the code comprising code to cause the processor to: (a) access a genomic library; (b) create an adjusted relative abundance proteomic library from the genomic library; (c) create a functionally characterized proteomic library from the adjusted relative abundance proteomic library; (d) supply a machine learning model with data from the functionally characterized proteomic library; and (e) determine, utilizing the machine learning model, a protein nutritional quality score for an organism from the genomic library. In some cases, the organism is a prokaryote, and the genomic library is a prokaryotic genomic library. In some cases, the organism is a eukaryote, and the genomic library is a eukaryotic genomic library. In some cases, the is a yeast, and the genomic library is a yeast genomic library. In some cases, the organism is a plant, and the genomic library is a plant genomic library.

In another aspect, provided herein is an in silico method for determining an organism's protein nutritional quality from a proteomic library, comprising: (a) accessing a proteomic library; (b) optionally creating an adjusted relative abundance proteomic library from the proteomic library; (c) creating a functionally characterized proteomic library from the adjusted relative abundance proteomic library; and (d) supplying a computational algorithm with data from the functionally characterized proteomic library, wherein the computational algorithm computes a protein nutritional quality score for an organism from the proteomic library. In some cases, the organism is a prokaryote, and the proteomic library is a prokaryotic proteomic library. In some cases, the organism is a eukaryote, and the proteomic library is a eukaryotic proteomic library. In some cases, the organism is a yeast, and the proteomic library is a yeast proteomic library. In some cases, the organism is a plant, and the proteomic library is a plant proteomic library. In some cases, the proteomic library comprises one or more protein amino acid sequences.

In another aspect, provided herein is a processor-readable non-transitory medium storing code representing instructions to be executed by a processor, the code comprising code to cause the processor to: (a) access a proteomic library; (b) create an adjusted relative abundance proteomic library from the proteomic library; (c) create a functionally characterized proteomic library from the adjusted relative abundance proteomic library; and (d) supply a computational algorithm with data from the functionally characterized proteomic library, wherein the computational algorithm computes a protein nutritional quality score for an organism from the proteomic library. In some cases, the organism is a prokaryote, and the proteomic library is a prokaryotic proteomic library. In some cases, the organism is a eukaryote, and the proteomic library is a eukaryotic proteomic library. In some cases, the organism is a yeast, and the proteomic library is a yeast proteomic library. In some cases, the organism is a plant, and the proteomic library is a plant proteomic library. In some cases, the proteomic library comprises one or more protein amino acid sequences.

In another aspect, provided herein is an in silico method for determining a microbial organism's protein nutritional quality from a microbial genomic library, comprising: (a) accessing a microbial genomic library; (b) creating an adjusted relative abundance microbial proteomic library from the microbial genomic library; (c) creating a functionally-characterized microbial proteomic library from the adjusted relative abundance microbial proteomic library; and (d) supplying a machine learning model with data from the functionally characterized microbial proteomic library, wherein the machine learning model computes a protein nutritional quality score for a microorganism from the microbial genomic library; and, wherein the method uses a mixture prediction algorithm to increase the average protein nutritional quality score of a composition by mixing one composition with a lower protein nutritional quality score with one or more compositions to improve the amino acid balance.

### BRIEF DESCRIPTION OF THE FIGURES

FIG. 1 shows a general schematic flow diagram of the PDCAAS pipeline.

FIG. 2 shows a general schematic flow diagram of the Expression Adjustment Module (EAP).

FIG. 3 shows graphically, Codon Adaptation Index (CAI) calculations using the PDCAAS pipeline compared to a previously published dataset (see Ishihama et al, BMC Genomics (2007), Vol 9(102):1-17). The X axis displays the pipeline calculated CAI and the Y axis displays the CAI from Ishihama et al.

FIG. 4 shows graphically a comparison of calculated E. coli protein expression using the PDCAAS pipeline compared to a previously published dataset, from Ishihama et al. The X axis displays the pipeline CAI and the Y axis displays the log(copy number) from Ishihama.

FIG. 5 shows graphically a comparison of amino acid percentages. The X axis displays the PDCAAS pipeline prediction and the Y axis displays the percentages from a previously published dataset (Jach et al, Metabolites, Vol 12(1):63).

FIG. 6 shows a general schematic flow diagram PDCAAS Calculation Module.

FIG. 7 shows graphically, a distribution of nutritional scores for Bacillus species. The X axis displays the strain number and the Y axis displays the ratio of most limiting amino acid (LRAAS).

FIG. 8 shows a ranking of organisms from the PDCAAS pipeline for their nutritional value. The X axis displays the organism's name and the Y axis displays the LRAAS.

FIG. 9 shows DIAAS scores calculated for various yeast and bacteria.

FIG. 10 shows graphically, the aromaticity distribution for all proteins in one organism.

FIG. 11 shows graphically, the distribution of molecular weights for one organism, Bacillus subtilis. The X axis displays the sequence identification (seq id) and the Y axis displays the protein molecular weight.

FIG. 12 shows graphically, the instability index for one organism, Bacillus subtilis. The X axis displays the sequence identification (seq id) and the Y axis displays the Instability Index.

FIG. 13 shows graphically, the isoelectric point distribution for one organism, Bacillus subtilis. The X axis displays the sequence identification (seq id) and the Y axis displays the Isoelectric point.

FIG. 14 shows graphically the gravy score for one organism. The X axis displays the sequence identification (seq id) and the Y axis displays the Gravy score.

FIG. 15 shows graphically, the secondary structure analysis for alpha helix, beta sheets, and the alpha helix/beta sheet ratio for Candida tropicalis, Kluyveromyces marxianus, Lactobacillus sanfrancicensis, Lipomyces starkeyi, and Saccharomyces cerevisiae.

FIG. 16 shows graphically, the training and testing loss per epoch.

FIG. 17 shows a confusion matrix for biLSTM training.

FIG. 18 shows graphically, an analysis of the Euclidean distance (X axis) and unadjusted PDCAAS (Y axis) of various microorganisms compared to various amino acid profiles of beef, lamb, liquid-egg-yolk, milk, pork, wheat flour, or whole egg.

FIG. 19 shows the Medallion Labs PDCAAS report for Kluyveromyces marxianus.

FIG. 20 shows the results for EAA percentage comparison between the Medallion report and the algorithm prediction for Kluyveromyces marxianus.

FIG. 21 shows graphically, the percentage of essential amino acids for two organisms.

FIG. 22 shows graphically, the amino acid profile for a mixture two organisms.

FIG. 23 shows a table of results from a Kraken analysis of taxonomic assignments based on sequencing of flow sorted samples: S004 and Sourdough.

FIG. 24 shows a general schematic flow diagram of the procedure for sequencing of fermented foods and analysis by nutritional algorithms.

FIG. 25 shows graphically, the distribution of PDCAAS scores for Gluten and Gliadin proteins.

FIG. 26 shows graphically, the distribution of amino acid scores for different organisms in the F+Y category.

### DETAILED DESCRIPTION

### Definitions

Unless defined otherwise herein, all technical and scientific terms used herein shall have meanings that are commonly understood by those of ordinary skill in the art to which the present disclosure belongs.

The term “genomic library” can refer generically to the concept of a database or other computer accessible file containing genetic sequences from an organism of interest. The term “genomic library” can also sometimes refer to the concept of a database or other computer accessible file containing genetic sequences from a plurality of organisms of interest. In some embodiments, the genomic libraries of the present disclosure may be embodied as: i) a collection of sequence information in a database or other computer file; ii) a sequenced whole genome from an organism in a database or other computer file; and iii) a partial whole genome sequence from an organism in a database or other computer file. The genomic library can be embodied in a public database containing genomic sequences for an organism, or a plurality of organisms, e.g. NCBI. The genomic library can be a privately created proprietary database containing sequence information from an organism or a plurality of organisms that has been created by Applicants.

The term “microorganism” includes, but is not limited to, bacteria, viruses, fungi, algae, yeasts, protozoa, worms, spirochetes, single-celled, and multi-celled organisms that are included in classification schema as prokaryotes, eukaryotes, archaea, and bacteria, and those that are known to those skilled in the art.

The term “plant” includes the class of higher and lower plants including angiosperms (monocotyledonous and dicotyledonous plants), gymnosperms, ferns, and multicellular algae. It includes plants of a variety of ploidy levels, including aneuploid, polyploid, diploid, haploid, and hemizygous.

The term “higher plant” refers to a large group of plants that have vascular tissues to distribute resources through the plant.

The term “computer” refers to a machine comprising a processor, a memory, and an operating system, capable of interaction with a user or other computer, and shall include without limitation desktop computers, notebook computers, laptop computers, processors, servers, personal digital assistants (PDAs), tablet computers, handheld computers, and similar devices that store data.

The term “cloud” may be used as a metaphor to refer to the internet and storing information, e.g. genomic sequences, in an off-site online system that is accessible via a computing device.

The term “in silico method” refers to a method of using a computer or computer algorithm to model a naturally occurring or in vitro process, and in some aspects to improve or predict a protein quality score.

The term “proteomic library” can refer generically to the concept of a database or other computer accessible file containing proteomic information and/or amino acid sequences from an organism of interest. The term “proteomic library” can also sometimes refer to the concept of a database or other computer accessible file containing proteomic information and/or amino acid sequences from a plurality of organisms of interest. In some embodiments, the proteomic libraries of the present disclosure may be embodied as: i) a collection of proteomic information and/or amino acid sequences in a database or other computer file; ii) a collection of proteomic information and/or amino acid sequences derived from a sequenced whole genome from an organism in a database or other computer file; and iii) a collection of proteomic information and/or amino acid sequences derived from a partial whole genome sequence from an organism in a database or other computer file. The proteomic library can be embodied in a public database containing proteomic expression information for an organism, or a plurality of organisms, e.g. NCBI. The proteomic library can be a privately created proprietary database containing proteomic information and/or amino acid sequences from an organism or a plurality of organisms that has been created by Applicants. In some embodiments, the proteomic library is obtained from methods known in the art including, but not limited to, mass spectrometry, protein chips, or reversed-phased protein microarrays.

The term “shotgun proteomics” refers to the use of bottom-up proteomics techniques in identifying proteins in complex mixtures using a combination of high-performance liquid chromatography combined with mass spectrometry. In shotgun proteomics, the spectra generated from all detectable proteins in a sample are interpreted by database searching. In contrast, targeted proteomic analysis is programmed to analyze a preselected group of proteins.

The term “prokaryote” refers to non-eukaryotic organisms belonging to the Eubacteria (e.g., Escherichia coli, Thermus thermophilus, etc.) and Archaea (e.g., Methanococcus jannaschii, Methanobacterium thermoautotrophicum, Halobacterium spp., A. fulgidus, P. firiosus, P. horikoshii, A. pernix, etc.) phylogenetic domains.

The term “eukaryote” refers to organisms belonging to the phylogenetic domain Eukarya such as animals (e.g., mammals, insects, reptiles, birds, etc.), ciliates, plants, fungi (e.g., yeasts, etc.), flagellates, microsporidia, protists, etc.

The term “unknown organism” refers to an organism having an unknown phenotype, an unknown genomic sequence, or lacking a scientific name. An unknown organism may be used for training, and samples of the unknown organism obtained elsewhere may be identified by the method. A system trained on an unknown organism may be used either to identify the unknown organism when it occurs in a sample, or to exclude the unknown organism. In an aspect, a plurality of unknown organisms may be used to train the system and subsequent samples may be categorized so as to select an organism from the plurality of unknown organisms. Some of the phenotypes may also be associated with known genotypes. In this manner, samples of, for example, soil, may be scanned so as to identify only those organisms of a single species, or only organisms of an unknown species of a group of unknown species.

The term “isolation sequencing” refers to a biological sample subjected to dilution with a growth medium and then incubated to promote isolation and growth of a desired microorganism. The resulting culture is then subjected to sequencing, which produces a sequencing result comprising genomic reads of only one specifically cultured organism. Accordingly, isolation sequencing can be used to identify and sequence an unknown organism. In some embodiments, isolation sequencing comprises obtaining results of metagenomics sequencing performed on biological samples from respective sample sources, identifying particular isolates of the biological samples from the metagenomics sequencing results, generating a genomic comparison component comprising hit abundance score vectors for respective ones of the identified samples. The systems and calculations disclosed herein can be trained and used on unknown organisms to determine their protein quality. The system can be further trained on new classes of unknown organisms as well if there is additional information such as differences in protein expression or digestibility.

The term “de novo sequencing” refers to a sensitive and unambiguous method to obtain information on sequence variations and organism identity. De novo sequencing may be used to determine the complete genome of an organism. The expression, via growth, of an organism's genes thereby creates a proteome. Therefore, de novo sequencing may be used by Applicants to obtain partial or whole proteomes. These genomes and proteomes may be wholly or partially novel, may represent unknown organisms, or may be used to update, improve, and correct previously known organisms. The algorithms described herein refer to both the in silico analysis of these proteomes and use in selection of organisms and hence physical instantiation of these proteomes.

The term “algorithm” can refer to a finite sequence of rigorous instructions, typically used to solve a class of specific problems or to perform a computation. Algorithms can be used as specifications for performing calculations and data processing. The basic elements of an algorithm can be sequence, selection, and iteration. The algorithm can be a step-by-step process, and the sequence of those steps may be crucial to ensuring the correctness of an algorithm. The algorithm can use selection to determine a different set of steps to execute based on a Boolean expression. Algorithms can further iterate to execute steps a certain number of times or until a certain condition is met.

The term “machine learning” can refer to the field of the computer sciences that studies the design of computer programs able to infer patterns, regularities, or rules from past experiences to develop an appropriate response to future data or describe the data in some meaningful way. By “machine learning” algorithms, in the context of this disclosure, it is meant association rule algorithms (e.g. a priori, discriminative pattern mining, frequent pattern mining, closed pattern mining, colossal pattern mining, and self-organizing maps), feature evaluation algorithms (e.g. information gain, Relief, ReliefF, RReliefF, symmetrical uncertainty, gain ratio, and ranker), subset selection algorithms (e.g. wrapper, consistency, classifier, correlation-based feature selection (CFS)), support vector machines, Bayesian networks, classification rules, decision trees, neural networks, instance-based algorithms, other algorithms that use the herein listed algorithms (e.g. vote, stacking, cost-sensitive classifier) and any other algorithm in the field of the computer sciences that relates to inducing patterns, regularities, or rules from past experiences to develop an appropriate response to future data. In some embodiments, a machine learning algorithm is PARROT. PARROT is a general framework for training and applying deep learning-based predictors on large protein datasets. PARROT is capable of tackling both classification and regression tasks by using an internal recurrent neural network architecture and only requires raw protein sequences as input. In some embodiments, a machine learning algorithm is Bidirectional Long Short-Term Memory, or BiLSTM. BiLSTM enables additional training by traversing the input data twice (i.e., 1. left-to-right and 2. right-to-left).

The term “PDCAAS” refers to a Protein Digestibility-Corrected Amino Acid Score (Schaafsma, Journal of AOAC International (2005), Vol 88(3):988-994). The traditional PDCAAS method is the one most commonly used today to estimate the protein quality of food intended for human consumption by providing a protein quality score. The PDCAAS evaluates the quality of proteins according to two criteria: the essential amino acid requirements of human beings and the digestibility of proteins.

The term “IVPDCAAS” refers to an in vitro Protein Digestibility-Corrected Amino Acid Score. The IVPDCAAS is based on the traditional PDCAAS method and incorporates in vitro tests as a surrogate for animal measurements. In one example, the PDCAAS-4-Enz method is shown to have a high R² (0.9649) when correlated with in vivo measurements, (Tavano et al, Food Research International (2016), Vol 89:756-763). This shows that in vivo measurements can be correlated with in vitro measurements, hence allowing in vitro measurements to be used in analyzing the accuracy of in silico analyses. The k-PDCAAS method is another method for accurate determination of in vitro PDCAAS and is used by labs such as Medallion Labs, see in vitro PDCAAS Megazyme kit protocol. U.S. Pat. No. 9,700,071B2 demonstrates that the maximum PDCAAS is 3.61.

The term “DIAAS” refers to Digestible Indispensable Amino Acid Score and is calculated as recommended by the Food and Agriculture Organization of the United Nations using equation DIAAS (%)=100 c lowest value of the DIAA reference ratio. For example, a score of <75% is a low protein quality score, 75-99% is a good protein quality score, and ≥100% is an excellent protein quality score. This in vivo DIAAS is normally performed in pigs.

The term “IVDIAAS” refers to an in vitro Digestible Indispensable Amino Acid Score. The IVDIAAS is based on the traditional DIAAS method and incorporates in vitro tests as a surrogate to animal measurements. In vitro DIAAS is performed via a simulated enzymatic digestion procedure and can be provided by labs such as Wageningen Food Research labs.

The term “R²” or “R-Squared” is a statistical measure that measures the amount of variance for a dependent variable that is explained by an independent variable in a regression model (Seeen.wikipedia.org/wiki/Coefficient_of_determination).

An “F test” or “F statistic” is a measure of a statistical model which measures the probability that the data fits the model (See en.wikipedia.org/wiki/F-test). It is typically measured by a p-value, which measures the probability that the results would be equally significant as the test results by random chance (See en.wikipedia.org/wiki/P-value).

The BLAST algorithm is the Basic Local Alignment Search Tool (Altschul et al, Journal of Molecular Biology (1990), Vol 215(3):403-410).

The term “Expression Adjusted Proteome” refers to the inference of protein levels based on sequence characteristics. This allows for an in silico prediction of the amino acid composition of the organism grown during production.

The term “amino acid score” (AAS) is a measure of the nutritional quality of a protein that may be calculated with the following formula: AAS=(mg of first limiting amino acid in 1 g test protein) divided by (mg of the same amino acid in 1 g reference protein).

The term “Amino Acid Profile” refers to the total protein, total nitrogen content, and percent of amino acids present in the sample. The measurement of total protein and nitrogen is performed using the Dumas method preferably, or alternatively the Kjeldahl method.

The term “Adjusted Proteome DataBank” (APDB) refers to a platform upon which machine learning can be performed to derive further analyses from expression adjustments, AAS scores, and other values reported by the platform. The platform can draw from a wide variety of public data sources, as well as store custom in-house sequenced and assembled genomes/proteomes, allowing for the storage of a large number of expression-adjusted and AAS-calculated proteomes within the APDB.

The term “Process Algorithm Layer” (PAL) can refer to inputs for downstream processing, baking processes and mixing. PAL can allow for prediction of protein quality properties at the end of a process. In some embodiments, alternative machine learning methods for PAL comprise a linear regression, a polynomial regression, a decision tree, a random forest, a biLSTM, a language model, and a CNN (Convolutional Neural Network).

### Overview

In one aspect, provided herein are in silico methods for determining the nutritional quality of proteins produced by an organism as well as processor-readable, non-transitory media for performing the aforementioned in silico methods. The processor-readable non-transitory media can store code representing instructions to be executed by a processor. In some cases, the in silico method can determine the nutritional quality of the proteins from the organism from a genomic library. In some cases, the in silico method can determine the nutritional quality of the proteins from the organism from a proteomic library. In some cases, the organism is a prokaryote, and the genomic library is a prokaryotic genomic library. In some cases, the organism is a eukaryote, and the genomic library is a eukaryotic genomic library. In some cases, the organism is a yeast, and the genomic library is a yeast genomic library. In some cases, the organism is a plant, and the genomic library is a plant genomic library. In some cases, the protein nutritional score can be a Protein Digestibility Corrected Amino Acid Score (PDCAAS), a Digestible Indispensable Amino Acid Score (DIAAS), an in vitro Protein Digestibility Corrected Amino Acid Score (IVPDCAAS), or an in vitro Digestible Indispensable Amino Acid Score (IVDIAAS). In some cases, the IVDIAAS score is at least 100. In some cases, the PDCAAS is at least 0.75. In some cases, the IVPDCAAS is at least 0.75. In some cases, the DIAAS is at least 75. In some cases, the IVDIAAS is at least 75. In some cases, the protein nutritional quality score is a Euclidean distance metric. In some cases, the target amino acid distribution is an amino acid distribution of proteins from milk. In some cases, the target amino acid distribution is an amino acid distribution of proteins from egg. In one embodiment, the in silico method for determining an organism's protein nutritional quality comprises: (a) accessing a genomic library; (b) creating an adjusted relative abundance proteomic library from the genomic library; (c) creating a functionally characterized proteomic library from the adjusted relative abundance proteomic library; and (d) supplying a computational algorithm with data from the functionally characterized proteomic library, wherein the computational algorithm computes a protein nutritional quality score for an organism from the genomic library.

Also provided herein are methods for selecting an organism as a source of protein. In some cases, the organism is a prokaryote, and the genomic library is a prokaryotic genomic library. In some cases, the organism is a eukaryote, and the genomic library is a eukaryotic genomic library. In some cases, the organism is a yeast, and the genomic library is a yeast genomic library. In some cases, the organism is a plant, and the genomic library is a plant genomic library. The protein sourced from an organism selected using the methods (e.g., in silico methods) provided herein can be used in a food product. Use in the food product can serve to improve the protein nutritional quality of the food product. The food product can be a human food product or animal food product. The animal food product can be a companion animal or farm animal food product. In some cases, the food product supplemented with protein sourced from an organism selected using the methods (e.g., in silico methods) provided herein can serve to improve the health of a subject who consumes said food product. In some cases, the subject can be a human or non-human animal. In some cases, the food product can improve one or more aspects of the subject's health such as, for example, muscle health, brain health, pregnancy health, elderly health, epilepsy, diabetes or cancer.

In one embodiment, the method for selecting an organism as a source of protein can comprise an in silico method that comprises: (a) accessing a genomic library comprising genomic information; (b) creating an adjusted relative abundance proteomic library from the genomic library; (c) creating a functionally-characterized proteomic library from the adjusted relative abundance proteomic library; (d) supplying a computational algorithm with data from the functionally characterized proteomic library; (e) computing a protein nutritional quality score with the computational algorithm; and (f) selecting an organism as a source of protein, wherein the computational algorithm selects the organism based on its computed protein nutritional quality score being above a desired threshold. In some cases, the genomic library comprises a plurality of nucleotide sequences (e.g., whole or partial genome) from a single organism. Further to this embodiment, the method can further comprise repeating steps (a)-(e) on a genomic library from one or more additional organisms, wherein step (f) entails selecting one or more organisms as the source of protein, wherein the computational algorithm selects the one or more organisms based on its computed protein nutritional quality score being above a desired threshold. In some cases, the genomic library comprises a plurality of nucleotide sequences (e.g., whole or partial genome) from a plurality of organisms. Further to this embodiment, steps (b)-(e) are performed for each organism from the plurality, while step (f) entails selecting one or more organisms as the source of protein, wherein the computational algorithm selects the one or more organisms based on their computed protein nutritional quality score being above a desired threshold. In some cases, the protein nutritional score can be a Protein Digestibility Corrected Amino Acid Score (PDCAAS), a Digestible Indispensable Amino Acid Score (DIAAS), an in vitro Protein Digestibility Corrected Amino Acid Score (IVPDCAAS), or an in vitro Digestible Indispensable Amino Acid Score (IVDIAAS). In some cases, the desired threshold can be an IVDIAAS score of at least 100. In some cases, the desired threshold can be a PDCAAS of at least 0.75. In some cases, the desired threshold can be a IVPDCAAS of at least 0.75. In some cases, the desired threshold can be a DIAAS of at least 75. In some cases, the desired threshold can be a IVDIAAS of at least 75. In some cases, the selected organism comprises a PDCAAS of at least 0.75. In some cases, the selected organism comprises a DIAAS of at least 75. In some cases, the protein nutritional quality score is a Euclidean distance metric. In some cases, the selected organism comprises a Euclidean distance less than 0.1 from a target amino acid distribution of 60% essential amino acids and 40% non-essential amino acids. In some cases, the selected organism comprises a Euclidean distance is less than 0.1 from a target amino acid distribution of 70% essential amino acids and 30% non-essential amino acids.

The computation algorithm for use in the methods provided herein can comprise one or more algorithms machine learning algorithm. In some cases, one of the one or more algorithms is a machine learning algorithm. The machine learning algorithm can further compute protein digestibility factors. In some cases, the protein digestibility factor is an alpha helix/beta-sheet ratio.

In some cases, the machine learning algorithm improves the accuracy of computing the digestibility factors.

In some cases, the protein nutritional quality score is a protein expression estimation score, a protein molecular weight calculation score, and/or an amino acid analysis score.

In some cases, creating the adjusted relative abundance proteomic library in a method provided herein comprises direct translation of the genomic library. Creating the adjusted relative abundance proteomic library can comprise direct translation of the microbial genomic library, and subsequent characterization of relative protein abundance. Creating the adjusted relative abundance proteomic library can comprise calculation of a codon adaptation index parameter for each protein in the library. Creating the adjusted relative abundance proteomic library can comprise calculation of a delta factor parameter comprising the Euclidean distance between each protein and the average ribosomal protein for each protein in the library. Creating the adjusted relative abundance proteomic library can comprise mass spectrometry based shotgun proteomics. Creating the functionally characterized proteomic library can comprise calculating one or more functional attributes of the library. Creating the functionally characterized proteomic library can comprise calculating one or more functional attributes selected from the group consisting of: overall amino acid composition, essential amino acid composition, non-essential amino acid composition, most limiting amino acid, and estimated nitrogen content.

In some cases, one or more modules of the computational algorithm in a method provided herein may utilize a machine learning method selected from the group consisting of linear regression, kernel ridge regression, logistic regression, neural networks, support vector machines, decision trees, hidden Markov models, Bayesian networks, a Gram-Schmidt process, reinforcement-based learning, self-supervised learning, cluster-based learning, hierarchical clustering, language models, bi-directional Long-Short-Term-Memory and genetic algorithms.

The genomic sequence can be obtained from de novo sequencing or isolation sequencing.

In some embodiments, the method uses a mixture prediction algorithm to increase the average protein nutritional quality score of a composition by mixing one composition with a lower protein nutritional quality score and one composition with a higher protein nutritional quality score.

In some embodiments, the method uses a mixture prediction algorithm to increase the average protein nutritional quality score of a composition by mixing a first, initial composition with a lower protein nutritional quality score and a second, initial composition with a higher protein nutritional quality score to increase the average protein nutritional quality score of a subsequent composition. In some embodiments, the first composition with a lower protein nutritional quality score comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20% about 21%, about 22%, about 23%, about 24%, about 25%, about 26%, about 27%, about 28%, about 29%, about 30% about 31%, about 32%, about 33%, about 34%, about 35%, about 36%, about 37%, about 38%, about 39%, about 40% about 41%, about 42%, about 43%, about 44%, about 45%, about 46%, about 47%, about 48%, about 49%, about 50% about 51%, about 52%, about 53%, about 54%, about 55%, about 56%, about 57%, about 58%, about 59%, about 60% about 61%, about 62%, about 63%, about 64%, about 65%, about 66%, about 67%, about 68%, about 69%, about 70% about 71%, about 72%, about 73%, about 74%, about 75%, about 76%, about 77%, about 78%, about 79%, about 80% about 81%, about 82%, about 83%, about 84%, about 85%, about 86%, about 87%, about 88%, about 89%, about 90% about 91%, about 92%, about 93%, about 94%, about 95%, about 96%, about 97%, about 98%, or about 99% of the subsequent composition.

In some embodiments, the second composition with a higher protein nutritional quality score comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20% about 21%, about 22%, about 23%, about 24%, about 25%, about 26%, about 27%, about 28%, about 29%, about 30% about 31%, about 32%, about 33%, about 34%, about 35%, about 36%, about 37%, about 38%, about 39%, about 40% about 41%, about 42%, about 43%, about 44%, about 45%, about 46%, about 47%, about 48%, about 49%, about 50% about 51%, about 52%, about 53%, about 54%, about 55%, about 56%, about 57%, about 58%, about 59%, about 60% about 61%, about 62%, about 63%, about 64%, about 65%, about 66%, about 67%, about 68%, about 69%, about 70% about 71%, about 72%, about 73%, about 74%, about 75%, about 76%, about 77%, about 78%, about 79%, about 80% about 81%, about 82%, about 83%, about 84%, about 85%, about 86%, about 87%, about 88%, about 89%, about 90% about 91%, about 92%, about 93%, about 94%, about 95%, about 96%, about 97%, about 98%, or about 99% of the subsequent composition.

In some embodiments, the subsequent composition comprises at least two initial compositions, at least three initial compositions, at least four initial compositions, at least five initial compositions, at least six initial compositions, at least seven initial compositions, at least eight initial compositions, at least nine initial compositions, or at least ten initial compositions.

### Description of the Algorithmic System

The method presented herein may be referred to as “algorithm”, “pipeline”, “software”, or “system”. The method can comprise a series of software modules that implement algorithms that may be traditional, bioinformatics algorithms, or machine learning algorithms.

The components of the software may be typically implemented in Python but can be implemented in multiple ways, including any variety of computer programming languages. The method may comprise tools, modules, or tables. Tools, modules, or tables in the system are written in lower case with underscores (e.g., tool: make_subsets). Below are various descriptions for different Tools, modules, and tables that can be used in the method:

### Tables

aas_scores—Relative scores for amino acids compared to a dietary reference, after expression adjustment.
bait proteins—Proteins used for bait analysis of highly expressed proteins in expression adjustment.
cai_results—CAI calculations for each protein in each proteome.
cds—Coding DNA Sequences for genes in each organism.
craaa_results—Cumulative Relative Amino Acid Abundance values for each amino acid in each organism.
digest_pred—Predicted secondary structure values for organisms.
digest_ref—Literature-based digestibility reference values.
eucl_dist—Euclidean distance results.
org_proteins—Protein sequences and accession numbers for each proteome in each organism.
pct_aa—Totals and percentages for amino acids for each organism.
pdcaas_ref—PDCAAS literature reference values for comparison to calculated values.
pdcaas_scores—Stores results of PDCAAS calculations; least represented amino acid, PDCAAS score, unadjusted PDCAAS for given organisms and dietary reference patterns.
protparam_scores—Data on physicochemical properties for each protein and organism.
ref_pattern—Profiles of dietary reference patterns.
ref_profile—Reference amino acid profiles for common foodstuffs for Euclidean distance calculations.
single_aas_scores—Amino acid scores for individual proteins.
single_protein_pdcaas_scores— PDCAAS scores for individual proteins.
wt_pct_scores—Molecular weight percentage scores for amino acids for organisms.

### Modules

build_index_set—Build a set of index proteins for CAI calculations.
calc_cai—Calculate expression estimates using CAI.
calc_little_d—Calculate the difference between the predicted PDCAAS and literature reference, referred to as “little d”.
craaa—Calculate the Cumulative Relative Amino Acid Abundance.
diaas_calc—Perform DIAAS calculations.
eucl_dist—Euclidean Distance calculations.
get_bait_proteins—Retrieve bait proteins used to find putative highly expressed proteins in the target organism.
make_mixture—Given two organisms and a percentage ratio, calculate the characteristics of their mixture.
pdcaas_calcs—Performs primary PDCAAS calculations.
pdcaas_pipeline—Main PDCAAS pipeline which performed expression estimation, molecular weight calculations, amino acid analysis, and PDCAAS estimation.
protparam—Calculate physicochemical properties of proteins.
single_protein_pdcaas—Calculate the PDCAAS for an individual protein sequence.

### Tools

get_ncbi_datasets—Uses the NCBI Datasets tool to retrieve large numbers of organisms for running in the PDCAAS pipeline.
jpred_pipeline—Generate training data using JPred API.
jpred_to_parrot—Convert JPred results to training files for PARROT.
load_baits—Given a file of bait protein sequences, register it with the system.
loadjasta—Generic loader for the FASTA file format.
load_proteome—Register and organism's proteome with the system.
make_subsets—Create subsets of proteomes for processing of organisms with larger genomes.
parse_jpred—Parse JPred results.
parse_parrot—Parse PARROT results for entry into digestibility tables.
process_pdcaas—Run PDCAAS pipeline against a large number of organisms.
process_single_proteins—Make PDCAAS calculations on sets of individual proteins.
register_blast_db—Register a BLAST database for an organism with the system.
register_files—Register sets for files for organism, these can then be processed by the process_pdcaas tool.

### Amino Acid Content

Maize, rice, and wheat are staple foods in many regions of the world; however, proteins from these grains are limited in certain amino acids, making their protein of poor dietary quality. For example, maize protein is limited in the amino acids lysine and tryptophan. Rice and wheat are additionally limiting in lysine. Consequently, populations that rely heavily on these foods may be lacking in at least one essential amino acid and require supplementation from other foods.

In some embodiments, the methods provided herein identify an organism with high protein quality that comprises all 20 amino acids: Histidine (H), Isoleucine (I), Leucine (L), Lysine (K), Methionine (M), Phenylalanine (F), Threonine (T), Tryptophan (W), Valine (V), Alanine (A), Arginine (R), Asparagine (N), Aspartic Acid (D), Glutamine (Q), Glutamic Acid (E), Glycine (G), Proline (P), Serine (S), Cysteine (C) and Tyrosine (Y).

Among the amino acids are non-essential amino acids (NEAAs), conditionally-essential amino acids (CEAA), and essential amino acids (EAA). Among the EAA are branched-chain amino acids (BCAAs). A BCAA is an amino acid having an aliphatic side-chain with a branch (a central carbon atom bound to three or more carbon atoms). BCAAs include leucine, isoleucine, and valine.

In some embodiments, the BCAA content of a protein product of the disclosure is between 20% to 25%, between 25% to 30%, between 30% to 35%, between 35% to 40%, between 40% to 45%, between 45% to 50%, between 50% to 55%, between 55% to 60%, between 60% to 65%, between 65% to 70%, between 70% to 75%, between 75% to 80%, between 80% to 85%, between 85% to 90%, between 90% to 95%, or between 95% to 100%.

In some embodiments, the BCAA content of a protein product of the disclosure is at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, or 100%.

In amino acid analysis, the amino acids Methionine (M) and Cysteine (C) cannot be distinguished because of their similarities. Thus, in the data reported here, as well as in the algorithm described herein, they are grouped into a category called “M+C”.

In amino acid analysis, the amino acids Phenylalanine (F) and Tyrosine (Y) cannot be distinguished because of their similarities. Thus, in the data reported here, as well as in the algorithm described herein, they are grouped into a category called “F+Y”.

### L-Amino Acids and D-Amino Acids

Amino acids are made from amine (—NH₂) and carboxylic acid (—COOH) functional groups, along with a side-chain specific to each type of amino acid. All amino acids (except glycine) can occur in two isomeric forms, because of the possibility of forming two different enantiomers (stereoisomers) around the central carbon atom. By convention, these stereoisomers are referred to as “L-” and “D-” forms, analogous to left-handed and right-handed configurations. Only L-amino acids are manufactured in cells and incorporated into proteins, whereas D-amino acids are more plentifully produced by microorganisms. The L-enantiomers of amino acids are widely assumed to account for most of their biological effects, including signaling, transporter-mediated protein interactions, and as a metabolic substrate. In mammals D-amino acids are believed to play a role in neuronal signaling. For example, D-serine is a potent ligand for the glycine binding site on the N-methyl-D-aspartate (NMDA) receptor. Other D-amino acids are present in the brain.

It is understood that a food product described herein may comprise both L-amino acids and D-amino acids. Unless stated otherwise, an amino acid as described herein described may comprise both L-amino acids and D-amino acids.

In some embodiments, a food product comprising a protein ingredient provided herein may comprise at least 0.0001% L-amino acids, at least 1% L-amino acids, at least 1% L-amino acids, at least 5% L-amino acids, at least 10% L-amino acids, at least 15% L-amino acids, at least 20% L-amino acids, at least 25% L-amino acids, at least 30% L-amino acids, at least 35% L-amino acids, at least 40% L-amino acids, at least 45% L-amino acids, at least 50% L-amino acids, at least 55% L-amino acids, at least 60% L-amino acids, at least 65% L-amino acids, at least 70% L-amino acids, at least 75% L-amino acids, at least 80% L-amino acids, at least 85% L-amino acids, at least 90% L-amino acids, at least 95% L-amino acids, or at least 99% L-amino acids.

In some embodiments, a protein ingredient provided herein or a food product comprising a protein ingredient provided herein may comprise between 0.0001% to 1% L-amino acids, between 1% to 5% L-amino acids, between 5% to 10% L-amino acids, between 10% to 15% L-amino acids, between 15% to 20% L-amino acids, between 20% to 25% L-amino acids, between 25% to 30% L-amino acids, between 30% to 35% L-amino acids, between 35% to 40% L-amino acids, between 40% to 45% L-amino acids, between 45% to 50% L-amino acids, between 50% to 55% L-amino acids, between 55% to 60% L-amino acids, between 60% to 65% L-amino acids, between 65% to 70% L-amino acids, between 70% to 75% L-amino acids, between 75% to 80% L-amino acids, between 80% to 85% L-amino acids, between 85% to 90% L-amino acids, between 90% to 95% L-amino acids, or between 95% to 99.9999% L-amino acids.

In some embodiments, a protein ingredient provided herein or a food product comprising a protein ingredient provided herein may comprise at least 0.0001% D-amino acids, at least 1% D-amino acids, at least 1% D-amino acids, at least 5% D-amino acids, at least 10% D-amino acids, at least 15% D-amino acids, at least 20% D-amino acids, at least 25% D-amino acids, at least 30% D-amino acids, at least 35% D-amino acids, at least 40% D-amino acids, at least 45% D-amino acids, at least 50% D-amino acids, at least 55% D-amino acids, at least 60% D-amino acids, at least 65% D-amino acids, at least 70% D-amino acids, at least 75% D-amino acids, at least 80% D-amino acids, at least 85% D-amino acids, at least 90% D-amino acids, at least 95% D-amino acids, or at least 99% D-amino acids.

In some embodiments, a protein ingredient provided herein or a food product comprising a protein ingredient provided herein may comprise between 0.0001% to 1% D-amino acids, between 1% to 5% D-amino acids, between 5% to 10% D-amino acids, between 10% to 15% D-amino acids, between 15% to 20% D-amino acids, between 20% to 25% D-amino acids, between 25% to 30% D-amino acids, between 30% to 35% D-amino acids, between 35% to 40% D-amino acids, between 40% to 45% D-amino acids, between 45% to 50% D-amino acids, between 50% to 55% D-amino acids, between 55% to 60% D-amino acids, between 60% to 65% D-amino acids, between 65% to 70% D-amino acids, between 70% to 75% D-amino acids, between 75% to 80% D-amino acids, between 80% to 85% D-amino acids, between 85% to 90% D-amino acids, between 90% to 95% D-amino acids, or between 95% to 99.9999% D-amino acids.

### Digestibility Factors

Digestibility factors can be measured by a variety of methods and represent the true ability of an organism to process the food material as it passes through the intestines. It may often be measured in animals after passage through the small intestine or through the animal (fecal matter analysis). Digestibility may or may not be affected by processing, drying, baking, or other downstream processes. Digestibility factors known for comparable materials can be used. For example, a comparable material, mycoprotein, is listed as having a digestibility of 0.86 (86%) (Edwards & Cummings, Proceedings of the Nutrition Society (2010), Vol. 69). The least represented AAS score was multiplied by the digestibility factor to arrive at the PDCAAS.

In some embodiments, a digestibility from mycoprotein or other factors derived from literature studies is used. In some embodiments, the digestibility factor may be from yeast-derived single cell protein, bacterial single cell protein, other single cell protein, plant-derived proteins, milk, whey, casein, or other protein standards known in the art. In some embodiments the digestibility factor may be derived from laboratory (in vitro) experiments or estimated using in vitro or in silico models.

In some embodiments, the PDCAAS algorithm calculates a digestibility factor, such as, for example, the number of beta sheets and/or alpha helices present. Digestibility may be positively correlated with number of alpha helices and the alpha helix/beta-sheet ratio. In some embodiments, the methods of the disclosure identify an organism with an alpha helix/beta-sheet ratio of about 0.001, about 0.005, about 0.0075, about 0.01, about 0.05, about 0.075, about 0.1, about 0.5, about 0.75, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 9.1, about 9.2, about 9.3, about 9.4, about 9.5, about 9.6, about 9.7, about 9.8, about 9.9, or about 10.

In some embodiments, the methods of the disclosure identify an organism with an alpha helix/beta-sheet ratio of from about 0.001 to about 0.01, from about 0.01 to about 0.1, from about 0.1 to about 1, about 1 to about 1.1, from about 1.1 to about 1.2, from about 1.2 to about 1.3, from about 1.3 to about 1.4, from about 1.4 to about 1.5, from about 1.5 to about 1.6, from about 1.6 to about 1.7, from about 1.7 to about 1.8, from about 1.8 to about 1.9, from about 1.9 to about 2, from about 2 to about 2.1, from about 2.1 to about 2.2, from about 2.2 to about 2.3, from about 2.3 to about 2.4, from about 2.4 to about 2.5, from about 2.5 to about 2.6, about 2.7, from about 2.7 to about 2.8, from about 2.8 to about 2.9, from about 2.9 to about 3, from about 3 to about 3.1, from about 3.1 to about 3.2, from about 3.2 to about 3.3, from about 3.3 to about 3.4, from about 3.4 to about 3.5, from about 3.5 to about 3.6, from about 3.6 to about 3.7, from about 3.7 to about 3.8, from about 3.8 to about 3.9, from about 3.9 to about 4, from about 4 to about 4.1, from about 4.1 to about 4.2, from about 4.2 to about 4.3, from about 4.3 to about 4.4, from about 4.4 to about 4.5, from about 4.5 to about 4.6, from about 4.6 to about 4.7, from about 4.7 to about 4.8, from about 4.8 to about 4.9, from about 4.9 to about 5, from about 5 to about 5.1, from about 5.1 to about 5.2, from about 5.2 to about 5.3, from about 5.3 to about 5.4, from about 5.4 to about 5.5, from about 5.5 to about 5.6, from about 5.6 to about 5.7, from about 5.7 to about 5.8, from about 5.8 to about 5.9, from about 5.9 to about 6, from about 6 to about 6.1, from about 6.1 to about 6.2, from about 6.2 to about 6.3, from about 6.3 to about 6.4, from about 6.4 to about 6.5, from about 6.5 to about 6.6, from about 6.6 to about 6.7, from about 6.7 to about 6.8, from about 6.8 to about 6.9, from about 6.9 to about 7, from about 7 to about 7.1, from about 7.1 to about 7.2, from about 7.2 to about 7.3, from about 7.3 to about 7.4, from about 7.4 to about 7.5, from about 7.5 to about 7.6, from about 7.6 to about 7.7, from about 7.7 to about 7.8, from about 7.8 to about 7.9, from about 7.9 to about 8, from about 8 to about 8.1, from about 8.1 to about 8.2, from about 8.2 to about 8.3, from about 8.3 to about 8.4, from about 8.4 to about 8.5, from about 8.5 to about 8.6, from about 8.6 to about 8.7, from about 8.7 to about 8.8, from about 8.8 to about 8.9, from about 8.9 to about 9, from about 9 to about 9.1, from about 9.1 to about 9.2, from about 9.2 to about 9.3, from about 9.3 to about 9.4, from about 9.4 to about 9.5, from about 9.5 to about 9.6, from about 9.6 to about 9.7, from about 9.7 to about 9.8, from about 9.8 to about 9.9, or about 10.

### Organism Growth Conditions

The conditions that an organism grows under can greatly affect the resulting protein's nutritional quality. In some embodiments, the growth conditions are tailored for an organism to enhance protein nutritional quality.

### Duration

In some embodiments, the organism is grown for at least about 1 minute, at least about 10 minutes, at least about 30 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 24 hours, at least about 48 hours, at least about 3 days at least about 4 days, at least about 5 days, at least about 6 days, at least about 7 days, at least about 1 week, at least about 2 weeks, at least about 3 weeks, at least about 4 weeks, at least about 1 months, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, or at least about 5 years.

In some embodiments, the organism is grown from about 1 minute to about 10 minutes, from about 30 minutes to about 1 hour, from about 1 hour to about 2 hours, from about 2 hours to about 3 hours, from about 3 hours to about 4 hours, from about 4 hours to about 5 hours, from about 5 hours to about 6 hours, from about 6 hours to about 7 hours, from about 7 hours to about 8 hours, from about 8 hours to about 9 hours, from about 9 hours to about 10 hours, from about 10 hours to about 11 hours, from about 11 hours to about 12 hours, from about 12 hours to about 24 hours, from about 24 hours to about 2 days, from about 2 days to about 3 days, from about 3 to about 4 days, from about 4 days to about 5 days, from about 5 days to about 6 days, from about 6 days to about 1 week, from about 1 week to about 2 weeks, from about 2 weeks to about 3 weeks, from about 3 weeks to about 4 weeks, from about 4 weeks to about 1 month, from about 1 month to about 2 months, from about 2 months to about 3 months, from about 3 months to about 4 months, from about 4 months to about 5 months, from about 5 months to about 6 months, from about 6 months to about 7 months, from about 7 months to about 8 months, from about 8 months to about 9 months, from about 9 months to about 10 months, from about 10 months to about 11 months, from about 11 months to about 1 year, from about 1 year to about 2 years, from about 2 years to about 3 years, from about 3 years to about 4 years, or from about 4 years to about 5 years.

### Temperature

In some embodiments, the organism is grown at about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., or about 100° C.

In some embodiments, the organism is grown from about 1° C. to about 2° C., from about 2° C. to about 3° C., from about 3° C. to about 4° C., from about 4° C. to about 5° C., from about 5° C. to about 6° C., from about 6° C. to about 7° C., from about 7° C. to about 8° C., from about 8° C. to about 9° C., from about 9° C. to about 10° C., from about 10° C. to about 11° C., from about 11° C. to about 12° C., from about 12° C. to about 13° C., from about 13° C. to about 14° C., from about 14° C. to about 15° C., from about 15° C. to about 16° C., from about 16° C. to about 17° C., from about 17° C. to about 18° C., from about 18° C. to about 19° C., from about 19° C. to about 20° C., from about 20° C. to about 21° C., from about 21° C. to about 22° C., from about 22° C. to about 23° C., from about 23° C. to about 24° C., from about 24° C. to about 25° C., from about 25° C. to about 26° C., from about 26° C. to about 27° C., from about 27° C. to about 28° C., from about 28° C. to about 29° C., from about 29° C. to about 30° C., from about 30° C. to about 31° C., from about 31° C. to about 32° C., from about 32° C. to about 33° C., from about 33° C. to about 34° C., from about 34° C. to about 35° C., from about 35° C. to about 36° C., from about 36° C. to about 37° C., from about 37° C. to about 38° C., from about 38° C. to about 39° C., from about 39° C. to about 40° C., from about 40° C. to about 41° C., from about 41° C. to about 42° C., from about 42° C. to about 43° C., from about 43° C. to about 44° C., from about 44° C. to about 45° C., from about 45° C. to about 46° C., from about 46° C. to about 47° C., from about 47° C. to about 48° C., from about 48° C. to about 49° C., from about 49° C. to about 50° C., from about 50° C. to about 51° C., from about 51° C. to about 52° C., from about 52° C. to about 53° C., from about 53° C. to about 54° C., from about 54° C. to about 55° C., from about 55° C. to about 56° C., from about 56° C. to about 57° C., from about 57° C. to about 58° C., from about 58° C. to about 59° C., from about 59° C. to about 60° C., from about 60° C. to about 61° C., from about 61° C. to about 62° C., from about 62° C. to about 63° C., from about 63° C. to about 64° C., from about 64° C. to about 65° C., from about 65° C. to about 66° C., from about 66° C. to about 67° C., from about 67° C. to about 68° C., from about 68° C. to about 68° C., from about 69° C. to about 70° C., from about 70° C. to about 71° C., from about 71° C. to about 72° C., from about 72° C. to about 73° C., from about 73° C. to about 74° C., from about 74° C. to about 75° C., from about 75° C. to about 76° C., from about 76° C. to about 77° C., from about 77° C. to about 78° C., from about 78° C. to about 79° C., from about 79° C. to about 80° C., from about 80° C. to about 81° C., from about 81° C. to about 82° C., from about 82° C. to about 83° C., from about 83° C. to about 84° C., from about 84° C. to about 85° C., from about 85° C. to about 86° C., from about 86° C. to about 87° C., from about 87° C. to about 88° C., from about 88° C. to about 89° C., from about 89° C. to about 90° C., from about 90° C. to about 91° C., from about 91° C. to about 92° C., from about 92° C. to about 93° C., from about 93° C. to about 94° C., from about 94° C. to about 95° C., from about 95° C. to about 96° C., from about 96° C. to about 97° C., from about 97° C. to about 98° C., from about 98° C. to about 99° C., or from about 99° C. to about 100° C.

Atmosphere

In some embodiments, the organism is grown in about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of oxygen (02) in the atmosphere.

In some embodiments, the organism is grown from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of oxygen (02) in the atmosphere.

In some embodiments, the organism is grown in about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of carbon dioxide (CO₂) in the atmosphere.

In some embodiments, the organism is grown from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of carbon dioxide (CO₂) in the atmosphere.

In some embodiments, the organism is grown in about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of nitrogen (N₂) in the atmosphere.

In some embodiments, the organism is grown from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of nitrogen (N₂) in the atmosphere.

### Calculations of Protein Nutritional Quality

In some embodiments, the present disclosure provides in silico methods to determine a microbial organism's protein nutritional quality from a microbial genomic library. In some cases, the microbial genomic library can comprise genomic information for an individual microbe. In some cases, the microbial genomic library can comprise genomic information for a plurality of microbes.

In some embodiments, an expression-adjusted proteome (EAP) incorporates several parameters to more accurately reflect the amino acid composition of the proteins produced by an organism. These parameters include, but are not limited to, codon usage and sequence characteristics.

In some embodiments, the Codon Adaptation Index (CAI) is used to predict higher or lower expression of proteins (Sharp & Li, Nucleic Acids Research (1987), Vol 15:1281-1295).

In some embodiments, the method of “delta” (Moura et al, Plos One (2013), Vol 8(10): e77319) or the method of Karlin (Karlin et al, J. Bacteriology (2001), Vol 183(17):5025-5040) are used to predict higher or lower expression of proteins.

In some embodiments, the Euclidean distance is used to calculate the amino acid distribution of the proteins produced by an organism.

The Euclidean distance of a given amino acid distribution (p) to a target distribution (t) is calculated using the following formula Distance=\sqrt{Σ_{iεAA}(pᵢ−tᵢ)²}, where AA={H, I, L, K, SAA, AAA, T, W, V, A, R, N, D, Q, E, G, P, and S}, pᵢ is the fraction of an amino acid in the given protein distribution, and t is the fraction of an amino acid in the target distribution. This metric corresponds to the square root of the sum of the squared error for each amino acid (EAAs, CEAAs, and NEAAs), and measures the percent distance between a given and target amino acid distribution.

In some embodiments, the methods of the disclosure identify an organism from a ratio of most limiting amino acid (LRAAS), nitrogen content, the levels of BCAA (Leucine, Isoleucine, Valine), Distribution of molecular weights, Aromaticity distribution, Instability index, Gravy score, Isoelectric point, and/or secondary structure characteristics.

In some embodiments, the methods of the disclosure identify an organism with a similar amino acid distribution compared to a target amino acid distribution as calculated by the Euclidean distance. In some embodiments, the target amino acid distribution is the amino acid distribution of a milk, pork, beef, lamb, or egg protein or protein distribution.

In some embodiments, the Euclidean distance is less than 0.2, less than 0.1, less than 0.05, less than 0.01 or less than 0.001 from a target amino acid distribution of 99% essential amino acids and 1% non-essential amino acids.

In some embodiments, the Euclidean distance is less than about 0.2 to about 0.1, less than about 0.1 to about 0.05, less than about 0.05 to about 0.01, or less than 0.01 to about 0.001 from a target amino acid distribution of 99% essential amino acids and 1% non-essential amino acids.

In some embodiments, the Euclidean distance is less than 0.2, less than 0.1, less than 0.05, less than 0.01 or less than 0.001 from a target amino acid distribution of 90% essential amino acids and 10% non-essential amino acids.

In some embodiments, the Euclidean distance is less than about 0.2 to about 0.1, less than about 0.1 to about 0.05, less than about 0.05 to about 0.01, or less than 0.01 to about 0.001 from a target amino acid distribution of 90% essential amino acids and 10% non-essential amino acids.

In some embodiments, the Euclidean distance is less than 0.2, less than 0.1, less than 0.05, less than 0.01 or less than 0.001 from a target amino acid distribution of 80% essential amino acids and 20% non-essential amino acids.

In some embodiments, the Euclidean distance is less than about 0.2 to about 0.1, less than about 0.1 to about 0.05, less than about 0.05 to about 0.01, or less than 0.01 to about 0.001 from a target amino acid distribution of 80% essential amino acids and 20% non-essential amino acids.

In some embodiments, the Euclidean distance is less than 0.2, less than 0.1, less than 0.05, less than 0.01 or less than 0.001 from a target amino acid distribution of 70% essential amino acids and 30% non-essential amino acids.

In some embodiments, the Euclidean distance is less than about 0.2 to about 0.1, less than about 0.1 to about 0.05, less than about 0.05 to about 0.01, or less than 0.01 to about 0.001 from a target amino acid distribution of 70% essential amino acids and 30% non-essential amino acids.

In some embodiments, the Euclidean distance is less than 0.2, less than 0.1, less than 0.05, less than 0.01 or less than 0.001 from a target amino acid distribution of 60% essential amino acids and 40% non-essential amino acids.

In some embodiments, the Euclidean distance is less than about 0.2 to about 0.1, less than about 0.1 to about 0.05, less than about 0.05 to about 0.01, or less than 0.01 to about 0.001 from a target amino acid distribution of 60% essential amino acids and 40% non-essential amino acids.

In some embodiments, the Euclidean distance is less than 0.2, less than 0.1, less than 0.05, less than 0.01 or less than 0.001 from a target amino acid distribution of 50% essential amino acids and 50% non-essential amino acids.

In some embodiments, the Euclidean distance is less than about 0.2 to about 0.1, less than about 0.1 to about 0.05, less than about 0.05 to about 0.01, or less than 0.01 to about 0.001 from a target amino acid distribution of 50% essential amino acids and 50% non-essential amino acids.

In some embodiments, the Euclidean distance is less than 0.2, less than 0.1, less than 0.05, less than 0.01 or less than 0.001 from a target amino acid distribution of 40% essential amino acids and 60% non-essential amino acids.

In some embodiments, the Euclidean distance is less than about 0.2 to about 0.1, less than about 0.1 to about 0.05, less than about 0.05 to about 0.01, or less than 0.01 to about 0.001 from a target amino acid distribution of 40% essential amino acids and 60% non-essential amino acids.

In some embodiments, the Euclidean distance is less than 0.2, less than 0.1, less than 0.05, less than 0.01 or less than 0.001 from a target amino acid distribution of 30% essential amino acids and 70% non-essential amino acids.

In some embodiments, the Euclidean distance is less than about 0.2 to about 0.1, less than about 0.1 to about 0.05, less than about 0.05 to about 0.01, or less than 0.01 to about 0.001 from a target amino acid distribution of 30% essential amino acids and 70% non-essential amino acids.

In some embodiments, the Euclidean distance is less than 0.2, less than 0.1, less than 0.05, less than 0.01 or less than 0.001 from a target amino acid distribution of 20% essential amino acids and 80% non-essential amino acids.

In some embodiments, the Euclidean distance is less than about 0.2 to about 0.1, less than about 0.1 to about 0.05, less than about 0.05 to about 0.01, or less than 0.01 to about 0.001 from a target amino acid distribution of 20% essential amino acids and 80% non-essential amino acids.

In some embodiments, the Euclidean distance is less than 0.2, less than 0.1, less than 0.05, less than 0.01 or less than 0.001 from a target amino acid distribution of 10% essential amino acids and 90% non-essential amino acids.

In some embodiments, the Euclidean distance is less than about 0.2 to about 0.1, less than about 0.1 to about 0.05, less than about 0.05 to about 0.01, or less than 0.01 to about 0.001 from a target amino acid distribution of 10% essential amino acids and 90% non-essential amino acids.

In some embodiments, the Euclidean distance is less than 0.2, less than 0.1, less than 0.05, less than 0.01 or less than 0.001 from a target amino acid distribution of 1% essential amino acids and 99% non-essential amino acids.

In some embodiments, the Euclidean distance is less than about 0.2 to about 0.1, less than about 0.1 to about 0.05, less than about 0.05 to about 0.01, or less than 0.01 to about 0.001 from a target amino acid distribution of 1% essential amino acids and 99% non-essential amino acids.

In some embodiments, the Euclidean distance of the amino acid distribution profile of a nutritive protein or fragment thereof is determined relative to a target amino acid distribution profile. In some embodiments the target amino acid distribution profile comprises a target distribution for EAAs. In some embodiments the target amino acid distribution profile comprises a target distribution for NEAAs. In some embodiments the target amino acid distribution profile comprises a target distribution for CEAAs. In some embodiments the target amino acid distribution profile comprises a target distribution for at least two of EAAs, CEAAs, and NEAAs. In some embodiments the target amino acid distribution profile comprises a target distribution for EAAs, CEAAs, and NEAAs. In some embodiments the target amino acid distribution profile of EAAs and CEAAs produces a desired PDCAAS score. In some embodiments the target amino acid distribution profile of NEAAs is the NEAA amino acid distribution profile of NEAAs present in a benchmark dietary protein source.

In some embodiments the target amino acid distribution profile of EAAs and CEAAs produces a PDCAAS score of at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2.0, at least about 2.1, at least about 2.2, at least about 2.3, at least about 2.4, at least about 2.5, at least about 2.6, at least about 2.7, at least about 2.8, at least about 2.9, at least about 3.0, at least about 3.1, at least about 3.2, at least about 3.3, at least about 3.4, at least about 3.5, or about 3.6. In some embodiments the target amino acid distribution profile of EAAs and CEAAs produces a PDCAAS score of at least about 0.8 to 1, at least about 1 to 1.2, at least about 1.2 to 1.4, at least about 1.4 to about 1.6, from about 1.4 to about 1.8, from about 1.4 to about 2.0, from about 1.4 to about 2.2, from about 1.4 to about 2.4, from about 1.4 to about 2.6, from about 1.4 to about 2.8, from about 1.4 to about 3.0, from about 1.4 to about 3.2, from about 1.4 to about 3.4, or from about 1.4 to about 3.6.

In some embodiments, the target amino acid distribution profile comprises at least 50% by weight EAAs. In some embodiments the target amino acid distribution profile comprises at least 55% by weight EAAs, at least 60% by weight EAAs, at least 65% by weight EAAs, at least 70% by weight EAAs, at least by weight 75% EAAs, at least 80% by weight EAAs, at least 85% by weight EAAs, at least 90% by weight EAAs, or at least 95% by weight EAAs, at least 96% by weight EAAs, at least 97% by weight EAAs, at least 98% by weight EAAs, at least 99% by weight EAAs, or 100% EAAs. In some embodiments the target amino acid distribution profile comprises from 50 to 100% by weight EAAs, from 60 to 100% by weight EAAs, from 70 to 100% by weight EAAs, from 80 to 90% by weight EAAs, from 60 to 90% by weight EAAs, from 60 to 80% by weight EAAs, from 70 to 90% by weight EAAs, from 60 to 70% by weight EAAs, from 70 to 80% by weight EAAs, from 80 to 90% by weight EAAs, and from 90 to 100% by weight EAAs. In some embodiments the target amino acid distribution profile comprises from 90% to 100% EAAs and CEAAs. That is, the combined fraction of essential amino acids and conditionally essential amino acids in the nutritive proteins is from 90% to 100%.

In some embodiments, the methods provided herein utilize a machine learning system, such as linear regression, polynomial regression, decision tree, neural network, language model, long-short-term memory and random forest, that can be continuously trained over time. The machine learning system can derive the test result for confirming or negating a protein nutritional quality score and incorporate it into training data. The training data can improve determination of a microbial organism's protein nutritional quality score generated by the machine learning system.

In some embodiments, these calculated protein nutritional quality scores allow for calculation of PDCAAS and DIAAS for many organisms, ranging from individual proteins, bacteria, lower eukaryotes such as yeast, through higher plants. These protein nutritional quality scores (e.g., PDCAAS and/or DIAAS) allow for determination of ideal organisms used to create protein for optimal nutritional quality.

In some embodiments, these calculated protein nutritional quality scores allow for calculation of IVPDCAAS and IVDIAAS for many organisms, ranging from an individual protein, bacteria, lower eukaryotes such as yeast, through higher plants. These scores allow for determination of ideal organisms used to create protein for optimal nutritional quality.

In some embodiments, these calculated protein nutritional quality scores allow for calculation of PDCAAS and DIAAS for many organisms, ranging from an individual protein, bacteria, lower eukaryotes such as yeast, through higher plants. These scores allow for determination of a microorganism protein nutritional quality score.

In some embodiments, these calculated protein nutritional quality scores allow for calculation of IVPDCAAS and IVDIAAS for many organisms, ranging from an individual protein, bacteria, lower eukaryotes such as yeast, through higher plants. These scores allow for determination of a microorganism protein nutritional quality score.

In some embodiments, the microorganism protein nutritional quality score may have a PDCAAS of at least 0.75, or at least 0.80, or at least 0.85, or at least 0.86, or at least 0.87, or at least 0.88, or at least 0.89, or at least 0.90, or at least 0.91, or at least 0.92, or at least 0.93, or at least 0.94, or at least 0.95, or at least 0.96, or at least 0.97, or at least 0.98, or at least 0.99 or at least 1.

In some embodiments, the microorganism protein nutritional quality score may have a PDCAAS of at least 1, or at least 1.25, or at least 1.5, or at least 1.75, or at least 2 or at least 2.25, or at least 2.5, or at least 2.75, or at least 3, or at least 3.25, or at least 3.5, or at least 3.6.

In some embodiments, the microorganism protein nutritional quality score may have a PDCAAS between 0.75 and 0.80, or between 0.80 and 0.85, or between 0.85 and 0.9, or between 0.90 and 0.92, or between 0.92 and 0.94, or between 0.94 and 0.96, or between 0.96 and 0.98, or between 0.98 and 1.

In some embodiments, the microorganism protein nutritional quality score may have a PDCAAS between 1 and 1.25, or between 1.25 and 1.50, or between 1.50 and 1.75, or between 1.75 and 2, or between 2 and 2.25, or between 2.25 and 2.50, or between 2.50 and 2.75, or between 2.75 and 3, or between 3 and 3.25, or between 3.25 and 3.50, or between 3.50 and 3.6.

In some embodiments, the method calculates a protein nutritional quality score that is comparable to in vitro data and in vivo data. That is, the in silico method calculates a protein nutritional quality score that provides an equivalent score that would be obtained from data obtained from animal studies or in vitro studies. In some embodiments, an equivalent score is a score that does not differ more than 1%, about 1%, about 2%, about 3%, about 4%, about 5%, about 6%, about 7%, about 8%, about 9%, about 10%, about 11%, about 12%, about 13%, about 14%, or about 15%. Therefore, the method calculates a protein nutritional quality score that has an equivalent IVPDCAAS and/or IVDIASS.

In some embodiments, the microorganism protein nutritional quality score may have an IVPDCAAS of at least 0.75, or at least 0.80, or at least 0.85, or at least 0.86, or at least 0.87, or at least 0.88, or at least 0.89, or at least 0.90, or at least 0.91, or at least 0.92, or at least 0.93, or at least 0.94, or at least 0.95, or at least 0.96, or at least 0.97, or at least 0.98, or at least 0.99 or at least 1.

In some embodiments, the microorganism protein nutritional quality score may have an IVPDCAAS of at least 1, or at least 1.25, or at least 1.5, or at least 1.75, or at least 2 or at least 2.25, or at least 2.5, or at least 2.75, or at least 3, or at least 3.25, or at least 3.5, or at least 3.6.

In some embodiments, the microorganism protein nutritional quality score may have an IVPDCAAS between 0.75 and 0.80, or between 0.80 and 0.85, or between 0.85 and 0.9, or between 0.90 and 0.92, or between 0.92 and 0.94, or between 0.94 and 0.96, or between 0.96 and 0.98, or between 0.98 and 1.

In some embodiments, the microorganism protein nutritional quality score may have a IVPDCAAS between 1 and 1.25, or between 1.25 and 1.50, or between 1.50 and 1.75, or between 1.75 and 2, or between 2 and 2.25, or between 2.25 and 2.50, or between 2.50 and 2.75, or between 2.75 and 3, or between 3 and 3.25, or between 3.25 and 3.50, or between 3.50 and 3.6.

In some embodiments, the microorganism protein nutritional quality score may have a DIAAS of at least 75, or at least 80, or at least 85, or at least 86, or at least 87, or at least 88, or at least 89, or at least 90, or at least 91, or at least 92, or at least 93, or at least 94, or at least 95, or at least 96, or at least 97, or at least 98, or at least 99 or at least 100.

In some embodiments, the microorganism protein nutritional quality score may have a DIAAS of at least 100, at least 110, at least 120, at least 130, at least 140, at least 150, at least 160, at least 170, at least 180, at least 190, or at least 200.

In some embodiments, the microorganism protein nutritional quality score may have a DIAAS of between 100 and 110, or between 110 and 120, or between 120 and 130, or between 130 and 140, or between 140 and 150, or between 150 and 160, or between 160 and 170, or between 170 and 180, or between 180 and 190, or between 190 and 200.

In some embodiments, the microorganism protein nutritional quality score may have a DIAAS between 75 and 80, or between 80 and 85, or between 85 and 90, or between 90 and 92, or between 92 and 94, or between 94 and 96, or between 96 and 98, or between 98 and 100.

In some embodiments, the microorganism protein nutritional quality score may have an IVDIAAS of at least 75, or at least 80, or at least 85, or at least 86, or at least 87, or at least 88, or at least 89, or at least 90, or at least 91, or at least 92, or at least 93, or at least 94, or at least 95, or at least 96, or at least 97, or at least 98, or at least 99 or at least 100.

In some embodiments, the microorganism protein nutritional quality score may have an IVDIAAS between 75 and 80, or between 80 and 85, or between 85 and 90, or between 90 and 92, or between 92 and 94, or between 94 and 96, or between 96 and 98, or between 98 and 100.

In some embodiments, the microorganism protein nutritional quality score may have an IVDIAAS of at least 100, at least 110, at least 120, at least 130, at least 140, at least 150, at least 160, at least 170, at least 180, at least 190, or at least 200.

In some embodiments, the microorganism protein nutritional quality score may have a IVDIAAS of between 100 and 110, or between 110 and 120, or between 120 and 130, or between 130 and 140, or between 140 and 150, or between 150 and 160, or between 160 and 170, or between 170 and 180, or between 180 and 190, or between 190 and 200.

In some embodiments, the nutritive protein comprises an individual protein sequence that comprises at least 20 amino acids. In some embodiments the individual protein sequence comprises at least 30 amino acids, at least 40 amino acids, at least 50 amino acids, at least 60 amino acids, at least 70 amino acids, at least 80 amino acids, at least 90 amino acids, at least 100 amino acids, at least 110 amino acids, at least 120 amino acids, at least 130 amino acids, at least 140 amino acids, at least 150 amino acids, at least 160 amino acids, at least 170 amino acids, at least 180 amino acids, at least 190 amino acids, at least 200 amino acids, at least 300 amino acids, at least 400 amino acids, at least 500 amino acids, at least 600 amino acids, at least 700 amino acids, at least 800 amino acids, at least 900 amino acids, or at least 1,000 amino acids. In some embodiments the individual protein sequence comprises from 20 to 50 amino acids, from 20 to 75 amino acids, from 20 to 100 amino acids, from 30 to 100 amino acids, from 40 to 100 amino acids, from 50 to 100 amino acids, from 50 to 200 amino acids, from 100 to 200 amino acids, from 200 to 300 amino acids, from 300 to 400 amino acids, or from 400 to 500 amino acids.

In some embodiments, the individual protein sequence comprises at least 50% by weight EAAs. In some embodiments, the individual protein sequence comprises at least 55% by weight EAAs, at least 60% by weight EAAs, at least 65% by weight EAAs, at least 70% by weight EAAs, at least by weight 75% EAAs, at least 80% by weight EAAs, at least 85% by weight EAAs, at least 90% by weight EAAs, or at least 95% by weight EAAs, at least 96% by weight EAAs, at least 97% by weight EAAs, at least 98% by weight EAAs, at least 99% by weight EAAs, or 100% EAAs.

In some embodiments the individual protein sequence comprises from 50 to 100% by weight EAAs, from 60 to 100% by weight EAAs, from 70 to 100% by weight EAAs, from 80 to 90% by weight EAAs, from 60 to 90% by weight EAAs, from 60 to 80% by weight EAAs, from 70 to 90% by weight EAAs, from 60 to 70% by weight EAAs, from 70 to 80% by weight EAAs, from 80 to 90% by weight EAAs, and from 90 to 100% by weight EAAs. In some embodiments the individual protein sequence comprises from 90% to 100% EAAs and CEAAs. In some embodiments the individual protein sequence comprises 100% EAAs and CEAAs.

In some embodiments, the present disclosure provides in silico methods to increase the protein nutritional quality score of a composition.

In some embodiments, the in silico method uses a mixture prediction algorithm to increase the average protein nutritional quality score of a composition by mixing one composition with a lower protein nutritional quality score and one composition with a higher protein nutritional quality score.

In some embodiments, the composition with a higher protein nutritional quality score is identified by an in silico method provided herein.

In some embodiments, the method identifies a species of yeast to increase the protein nutritional quality score of the composition.

In some embodiments, the method identifies a species of bacteria to increase the protein nutritional quality score of the composition.

In some embodiments, the method identifies a species of fungi to increase the protein nutritional quality score of the composition.

In some embodiments, the method identifies a species of plant to increase the protein nutritional quality score of the composition.

In some embodiments, the method identifies an unknown organism to increase the protein nutritional quality score of the composition.

In some embodiments, the method identifies individual proteins to increase the protein nutritional quality score of the composition.

In some embodiments, the average protein nutritional quality score of the composition may have a PDCAAS of at least 0.75, or at least 0.80, or at least 0.85, or at least 0.86, or at least 0.87, or at least 0.88, or at least 0.89, or at least 0.90, or at least 0.91, or at least 0.92, or at least 0.93, or at least 0.94, or at least 0.95, or at least 0.96, or at least 0.97, or at least 0.98, or at least 0.99 or at least 1.

In some embodiments, the average protein nutritional quality score of the composition may have a PDCAAS between 0.75 and 0.80, or between 0.80 and 0.85, or between 0.85 and 0.9, or between 0.90 and 0.92, or between 0.92 and 0.94, or between 0.94 and 0.96, or between 0.96 and 0.98, or between 0.98 and 1.

In some embodiments, the average protein nutritional quality score of the composition may have an IVPDCAAS of at least 0.75, or at least 0.80, or at least 0.85, or at least 0.86, or at least 0.87, or at least 0.88, or at least 0.89, or at least 0.90, or at least 0.91, or at least 0.92, or at least 0.93, or at least 0.94, or at least 0.95, or at least 0.96, or at least 0.97, or at least 0.98, or at least 0.99 or at least 1.

In some embodiments, the average protein nutritional quality score of the composition may have an IVPDCAAS between 0.75 and 0.80, or between 0.80 and 0.85, or between 0.85 and 0.9, or between 0.90 and 0.92, or between 0.92 and 0.94, or between 0.94 and 0.96, or between 0.96 and 0.98, or between 0.98 and 1.

In some embodiments, the average protein nutritional quality score of the composition may have a PDCAAS of at least 1, or at least 1.25, or at least 1.5, or at least 1.75, or at least 2 or at least 2.25, or at least 2.5, or at least 2.75, or at least 3, or at least 3.25, or at least 3.6.

In some embodiments, the average protein nutritional quality score of the composition may have a PDCAAS between 1 and 1.25, or between 1.25 and 1.50, or between 1.50 and 1.75, or between 1.75 and 2, or between 2 and 2.25, or between 2.25 and 2.50, or between 2.50 and 2.75, or between 2.75 and 3, or between 3 and 3.25, or between 3.25 and 3.50, or between 3.50 and 3.6.

In some embodiments, the average protein nutritional quality score of the composition may have an IVPDCAAS of at least 1, or at least 1.25, or at least 1.5, or at least 1.75, or at least 2 or at least 2.25, or at least 2.5, or at least 2.75, or at least 3, or at least 3.25, or at least 3.5, or at least 3.75, or at least 4.

In some embodiments, the average protein nutritional quality score of the composition may have an IVPDCAAS between 1 and 1.25, or between 1.25 and 1.50, or between 1.50 and 1.75, or between 1.75 and 2, or between 2 and 2.25, or between 2.25 and 2.50, or between 2.50 and 2.75, or between 2.75 and 3, or between 3 and 3.25, or between 3.25 and 3.50, or between 3.50 and 3.75, or between 3.75 and 4. In some embodiments, the average protein nutritional quality score of the composition may have a DIAAS of at least 100, at least 110, at least 120, at least 130, at least 140, at least 150, at least 160, at least 170, at least 180, at least 190, or at least 200.

In some embodiments, the average protein nutritional quality score of the composition may have a DIAAS of between 100 and 110, or between 110 and 120, or between 120 and 130, or between 130 and 140, or between 140 and 150, or between 150 and 160, or between 160 and 170, or between 170 and 180, or between 180 and 190, or between 190 and 200.

In some embodiments, the average protein nutritional quality score of the composition may have a DIAAS between 75 and 80, or between 80 and 85, or between 85 and 90, or between 90 and 92, or between 92 and 94, or between 94 and 96, or between 96 and 98, or between 98 and 100.

In some embodiments, the average protein nutritional quality score of the composition may have an IVDIAAS of at least 75, or at least 80, or at least 85, or at least 86, or at least 87, or at least 88, or at least 89, or at least 90, or at least 91, or at least 92, or at least 93, or at least 94, or at least 95, or at least 96, or at least 97, or at least 98, or at least 99 or at least 100.

In some embodiments, the average protein nutritional quality score of the composition may have an IVDIAAS between 75 and 80, or between 80 and 85, or between 85 and 90, or between 90 and 92, or between 92 and 94, or between 94 and 96, or between 96 and 98, or between 98 and 100.

In some embodiments, the average protein nutritional quality score of the composition may have a IVDIAAS of at least 100, at least 110, at least 120, at least 130, at least 140, at least 150, at least 160, at least 170, at least 180, at least 190, or at least 200.

In some embodiments, the average protein nutritional quality score of the composition may have a IVDIAAS of between 100 and 110, or between 110 and 120, or between 120 and 130, or between 130 and 140, or between 140 and 150, or between 150 and 160, or between 160 and 170, or between 170 and 180, or between 180 and 190, or between 190 and 200.

In some embodiments, the composition comprises at least 50% by weight EAAs. In some embodiments, the composition comprises at least 55% by weight EAAs, at least 60% by weight EAAs, at least 65% by weight EAAs, at least 70% by weight EAAs, at least by weight 75% EAAs, at least 80% by weight EAAs, at least 85% by weight EAAs, at least 90% by weight EAAs, or at least 95% by weight EAAs, at least 96% by weight EAAs, at least 97% by weight EAAs, at least 98% by weight EAAs, at least 99% by weight EAAs, or 100% EAAs. In some embodiments, the composition comprises from 50 to 100% by weight EAAs, from 60 to 100% by weight EAAs, from 70 to 100% by weight EAAs, from 80 to 90% by weight EAAs, from 60 to 90% by weight EAAs, from 60 to 80% by weight EAAs, from 70 to 90% by weight EAAs, from 60 to 70% by weight EAAs, from 70 to 80% by weight EAAs, from 80 to 90% by weight EAAs, and from 90 to 100% by weight EAAs. In some embodiments, the composition comprises from 90% to 100% EAAs and CEAAs. In some embodiments, the composition comprises 100% EAAs and CEAAs.

In some embodiments, an algorithmic layer incorporates inputs for downstream processing, baking processes, and mixing. These inputs include, but are not limited to, reference gene index set, CAI method (e.g., Sharp & Li, Nucleic Acids Research (1987), Vol 15:1281-1295, delta, Karlin et al, J. Bacteriology (2001), Vol 183(17):5025-5040), protein copy number function (e.g., exponential), class of organism, APDB information, physicochemical properties, digestibility factors, downstream processing methods (drying time, heating, etc.), proteolysis and enzymes used, baking methods used (heat, time, etc.), and additional ingredients.

### Fermented Foods

Microorganisms can be isolated from a wide variety of locations, including fermented foods. These foods can provide organisms that are ideal for consumption of starch compounds in foods, and often provide flavor and other components in the resulting food product. A great deal of information on microorganisms from fermented foods and their omics studies can be found for example in ODFM data source (Whon et al, Scientific Data (2021), Vol 8(113):1-10).

Food Products Derived from Microbial Ingredients

In some embodiments, the method and/or algorithm of the present disclosure is used to select microbes for fermentation processes to produce microbial protein ingredients for food applications. Microbial ingredients may be formulated into doughs, soups or powders (U.S. 63/428,014) for incorporation into foods. This method increases the protein content of traditional foods (U.S. 63/419,237).

Animal sources tend to have more complete protein and higher PDCAAS than plant proteins (Ismail et al, Animal Frontiers (2020), Vol 10(4):53-63). Microbial proteins present an important opportunity to add complete proteins into foods without the production of animal resources.

Foods containing Microbial Protein (MP), also called Single Cell Protein (SCP) could significantly improve protein availability for the world's population (Ritala et al, Frontiers in Microbiology (2017), Vol 8), while reducing the environmental impact of meat production (Humpenoder et al, Nature (2022), Vol 605:90-96). MP, besides being high in protein, also contains fats, carbohydrates, vitamins, and minerals (Sheth & Patel, Food Microbiology Based Entrepreneurship (2023) pp 133-152) and tends to be rich in amino acids lysine and methionine which are often lacking in plant-based protein sources. Foods containing MP can be an important source of dietary fiber (Salazer-Lopez et al, Bioengineering (2022), Vol 9(11):623). Yeast can be an important source of B vitamins as well. Microbial proteins may represent a healthier alternative to meat proteins, potentially reducing toxins in the gut, improving gut microbiota and gut health, and reducing the risk of colorectal cancer (Farsi et al, European Journal of Nutrition (2023)).

In some embodiments, the method and/or algorithm of the present disclosure is used to select microbes that can be used in foods containing MP that can be useful in companion and farm animal health. Foods high in MP can provide benefits to companion animal health similar to those found in Humans. Use of MP in animal feeds can contribute to animal health and reduce greenhouse gas emissions (Shreck et al, Journal of Animal Science (2021), Vol 99(7): skab147). MP is also useful in aquaculture (Jones et al, Current Opinion in Biotechnology (2020), Vol 61:189-197).

Animal Food Products

In some embodiments, the methods of the disclosure selects an organism to produce protein ingredients for use in a companion animal food product. In some embodiments, the organism improves the protein quality of the food product. In some embodiments, the methods of the disclosure selects an organism to produce protein ingredients for use in a farm animal food product. In some embodiments, the organism improves the protein quality of the food product.

Providing high quality protein for animals has posed a significant challenge to ensure sustained animal health. For example, insufficient or poor protein quality can aggravate the age-associated loss of lean body mass and may contribute to earlier mortality in companion animals. (See Laflamme, Top Companion Anim Med., 23(3):154-7 (2008)). Additionally, the use of certain food products from animals, such as feather meal products, in the food system may result in human and animal exposure to arsenic due to the use of arsenic-based antibiotics in the poultry industry (See Nachman et al. Sci Total Environ 15; 417-418 (2012)). Moreover, supplementing animal diets with high quality protein has benefits for both animal health and environmental health.

The term “farm animal” can include livestock raised for use and/or profit. The term “farm animal” can include, but are not limited to, fish, cattle, sheep, pigs, goats, horses, donkeys, mules, and poultry (e.g. chickens, ducks, turkeys, and geese).

The term “companion animal” can include pet animals kept primarily for a person's company or entertainment. The term companion animal can include, but are not limited to, a dog, a cat, or a bird, such as a parrot.

In some embodiments, the companion animal food product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of protein from the selected organism.

In some embodiments, the companion animal food product comprises from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of protein from the selected organism.

In some embodiments, the farm animal food product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of protein from the selected organism.

In some embodiments, the farm animal food product comprises from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of protein from the selected organism.

Human Food Products

In some embodiments, the methods of the disclosure selects an organism to produce protein ingredients for use in a human food product. In some embodiments, the organism improves the protein quality of the food product.

Protein in an important component of the human diet and supports growth and well-being. Insufficient protein can lead to low growth and a weekend immune system. Therefore, supplementing human food products with protein can support growth and well-being.

In some embodiments, the human food product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of protein from the selected organism.

In some embodiments, the human food product comprises from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of protein from the selected organism.

### Muscle Health

In some embodiments, the methods of the disclosure select an organism to produce protein ingredients for use in a human food product. In some embodiments, the organism improves leucine content in a food product designed to support increased muscle.

Skeletal muscle is the largest organ in the human body, representing ˜40% of the total body weight, which stores energy in the form of proteins (amino acids). The skeletal muscle exhibits plasticity in response to the environment; proper exercise combined with adequate nutrition leads to muscle hypertrophy. Central to muscle growth is proper nutrition, including a diet right in protein. Leucine has been shown to stimulate muscle growth. (See Kamei, Nutrients (2020), Vol 12(1):261).

In some embodiments, the food product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of leucine.

In some embodiments, the food product comprises from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of leucine.

Brain Health

In some embodiments, the methods of the disclosure select an organism to produce protein ingredients for use in a human food product. In some embodiments, the organism improves tryptophan content in a food product designed to support brain health.

Tryptophan is an essential component of the diet, plays a key role in protein synthesis, and is a precursor of biologically active compounds such as serotonin. Studies have shown that demonstrated a relationship between the amount of serotonin synthesized in the brain and the amount of tryptophan supplied to the body with diet. Additionally, tryptophan availability to the brain is low, especially in subjects with depression. (See Keszthelyi Am J Clin Nutr. (2012) 95:603-08 and Kaluzna-Czaplinksa, Critical Reviews in Food Science & Nutrition (2019), Vol 59(1):72-88). Consequently, increased diet tryptophan may support brain health.

In some embodiments, the food product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of tryptophan.

In some embodiments, the food product comprises from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of tryptophan.

### Pregnancy Health

In some embodiments, the methods of the disclosure select an organism to produce protein ingredients for use in a human food product. In some embodiments, the organism improves lysine and/or phenylalanine content in a food product designed to support pregnancy health.

During pregnancy, amino acids are important biomolecules that play essential roles in fetal growth and development. Imbalanced amino acid intake during gestation may produce long-term morphological or functional changes in offspring, for example, developmental programming that increases the risk of developing hypertension in later life. Moreover, studies have shown that lysine requirements during late gestation increase by 27% and that there is a 40% higher requirement for phenylalanine during late gestation. (See Payne et al. J. Nutr. (2018) 148:94-99 and Ennis et al. Am. J. Clin. Nutr. (2020) 111:351-359). Consequently, supplementing diet with lysine and/or phenylalanine could support pregnancy health.

In some embodiments, the food product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of lysine.

In some embodiments, the food product comprises from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of lysine.

In some embodiments, the food product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of phenylalanine.

In some embodiments, the food product comprises from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of phenylalanine.

### Elderly Health

In some embodiments, the methods of the disclosure select an organism to produce protein ingredients for use in a human food product. In some embodiments, the organism improves EAA content in a food product designed to support elderly health.

As humans age, so do dietary requirements. Studies have identified that diet supplementation of EAA in aging individuals improves quality of life in elderly patients. (See Rondanelli et al, Clinical Nutrition (2011), Vol 30(5):571-577). Therefore, supplementing food products with EAA for aging individuals may improve quality of life.

In some embodiments, the food product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of EAA.

In some embodiments, the food product comprises from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of EAA.

### Epilepsy

In some embodiments, the methods of the disclosure select an organism to produce protein ingredients for use in a human food product. In some embodiments, the organism improves ketogenic amino acid and/or D-leucine content in a food product designed to reduce seizures.

The ketogenic diet (KD) has been identified as a useful therapy for antiepileptic drug-resistant epilepsy. One possible strategy for enhancing the efficacy of the KD therapy is modifying the diet with the ketogenic amino acids, leucine and lysine, as opposed to glucogenic amino acids (See Takeuchi et al., Front. Neurosci. (2021) Vol. 15:637288). Moreover, studies have shown that diet supplementation with D-leucine effectively terminated seizures, even at low doses (See Hartman et al., Neurobiol Dis. (2015) Vol. 82:46-53).

In some embodiments, a food product comprises ketogenic amino acids. In some embodiments, the food product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of ketogenic acids.

In some embodiments, the food product comprises from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of ketogenic amino acids.

In some embodiments, the food product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of D-leucine.

In some embodiments, the food product comprises from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of D-leucine.

### Diabetes

In some embodiments, the methods of the disclosure select an organism to produce protein ingredients for use in a human food product. In some embodiments, the organism decreases BCAA content in a food product designed to support diabetic health.

Increased circulating levels of BCAAs have been linked with increased insulin resistance, increased occurrence of metabolic syndrome and type 2 diabetes (T2DM). Therefore, decreasing EAA in food products for diabetic individuals may improve quality of life.

In some embodiments, the food product comprises less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, about 42%, about 43%, about 44%, about 45%, about 46%, about 47%, about 48%, about 49%, about 50%, about 51%, less than about 52%, less than about 53%, less than about 54%, less than about 55%, less than about 56%, less than about 57%, less than about 58%, less than about 59%, less than about 60%, less than about 61%, less than about 62%, less than about 63%, less than about 64%, less than about 65%, less than about 66%, less than about 67%, less than about 68%, less than about 69%, less than about 70%, less than about 71%, less than about 72%, less than about 73%, less than about 74%, less than about 75%, less than about 76%, less than about 77%, less than about 78%, less than about 79%, less than about 80%, less than about 81%, less than about 82%, less than about 83%, less than about 84%, less than about 85%, less than about 86%, less than about 87%, less than about 88%, less than about 89%, less than about 90%, less than about 91%, less than about 92%, less than about 93%, less than about 94%, less than about 95%, less than about 96%, less than about 97%, less than about 98%, less than about 99%, or less than about 100% of BCAA.

### Cancer

In some embodiments, the methods of the disclosure select an organism to produce protein ingredients for use in a human food product. In some embodiments, the organism improves BCAA content in a food product designed to support cancer patients.

Nutrition for cancer patients is crucial to monitor because the cancer may preferentially consume certain nutrients, such as BCAA, and subsequently starve healthy cells. (See Lee & Blanton, Nutrition & Health (2023), Vol 0(0)). Therefore, increasing BCAA in food products for cancer patients may improve quality of life.

In some embodiments, the food product comprises about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of BCAA.

In some embodiments, the food product comprises from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10%, from about 10% to about 11%, from about 11% to about 12%, from about 12% to about 13%, from about 13% to about 14%, from about 14% to about 15%, from about 15% to about 16%, from about 16% to about 17%, from about 17% to about 18%, from about 18% to about 19%, from about 19% to about 20%, from about 20% to about 21%, from about 21% to about 22%, from about 22% to about 23%, from about 23% to about 24%, from about 24% to about 25%, from about 25% to about 26%, from about 26% to about 27%, from about 27% to about 28%, from about 28% to about 29%, from about 29% to about 30%, from about 30% to about 31%, from about 31% to about 32%, from about 32% to about 33%, from about 33% to about 34%, from about 34% to about 35%, from about 35% to about 36%, from about 36% to about 37%, from about 37% to about 38%, from about 38% to about 39%, from about 39% to about 40%, from about 40% to about 41%, from about 41% to about 42%, from about 42% to about 43%, from about 43% to about 44%, from about 44% to about 45%, from about 45% to about 46%, from about 46% to about 47%, from about 47% to about 48%, from about 48% to about 49%, from about 49% to about 50%, from about 50% to about 51%, from about 51% to about 52%, from about 52% to about 53%, from about 53% to about 54%, from about 54% to about 55%, from about 55% to about 56%, from about 56% to about 57%, from about 57% to about 58%, from about 58% to about 59%, from about 59% to about 60%, from about 60% to about 61%, from about 61% to about 62%, from about 62% to about 63%, from about 63% to about 64%, from about 64% to about 65%, from about 65% to about 66%, from about 66% to about 67%, from about 67% to about 68%, from about 68% to about 68%, from about 69% to about 70%, from about 70% to about 71%, from about 71% to about 72%, from about 72% to about 73%, from about 73% to about 74%, from about 74% to about 75%, from about 75% to about 76%, from about 76% to about 77%, from about 77% to about 78%, from about 78% to about 79%, from about 79% to about 80%, from about 80% to about 81%, from about 81% to about 82%, from about 82% to about 83%, from about 83% to about 84%, from about 84% to about 85%, from about 85% to about 86%, from about 86% to about 87%, from about 87% to about 88%, from about 88% to about 89%, from about 89% to about 90%, from about 90% to about 91%, from about 91% to about 92%, from about 92% to about 93%, from about 93% to about 94%, from about 94% to about 95%, from about 95% to about 96%, from about 96% to about 97%, from about 97% to about 98%, from about 98% to about 99%, or from about 99% to about 100% of BCAA.

The present disclosure is further illustrated by reference to the following Examples. However, it should be noted that these Examples, like the embodiments described above, are illustrative and are not to be construed as restricting the scope of the disclosure in any way.

### EXAMPLES

### Example 1: Calculation of Expression-Adjusted Proteome (EAP)

### Purpose

The purpose of this example was to determine if the PDCAAS pipeline provides an accurate estimate of the true protein copy number in the cell.

### Methods

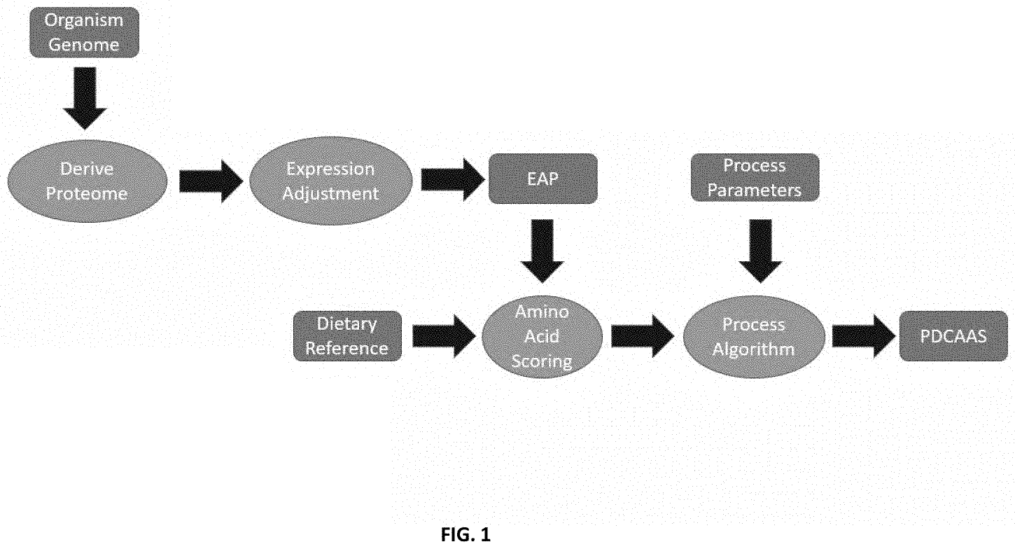

The PDCAAS pipeline first calculated an expression-adjusted proteome and then proceeded to molecular weight calculations, digestibility, and finally PDCAAS estimation. An overview of the entire pipeline is shown in FIG. 1.

First, the relative ratios of various proteins were adjusted, and hence amino acids, based on expression levels. Within the genome, sections coding for proteins (Coding DNA Sequences, CDS) are first transcribed into mRNA, and then translated into proteins by ribosomes. A three-letter nucleotide code (the codon) specifies which amino acid is used. A transfer RNA (tRNA) connects the 3-letter code to the amino acid.

During protein translation, more abundant proteins use codons that represent greater abundance of tRNAs, allowing for higher levels of those proteins to be made. Codon usage has generally been found to correlate well with protein expression levels (Wang et al, Genome Research (2005), Vol 15(8):1118-1126). It has been found that the Codon Adaptation Index (CAI) can be used to predict higher or lower expression of proteins.

Here, the CAI index was used, as well as related methods, to predict the expression of proteins within a genome. This allowed for prediction of the relative amino acid levels and percentages of each amino acid expected to be present in each proteome starting only with a genome.

The method of CAI was first examined by Sharp and Li (Sharp & Li, Nucleic Acids Research (1987), Vol 15:1281-1295). It has been shown that the CAI has a good correlation with actual protein abundance measurements.

In this method, a standard reference set of known highly expressed proteins (ribosomal proteins) was used and then a CAI for adjusting the relative protein expression was calculated. One may also use, alternatively, the method of “delta” or the method of Karlin (Karlin et al, J. Bacteriology (2001), Vol 183(17):5025-5040), which provides similar results.

Another method that may be used to calculate CAI is machine learning (Ferreira et al, Journal of Molecular Biology (2021), Vol 433(22), 167267).

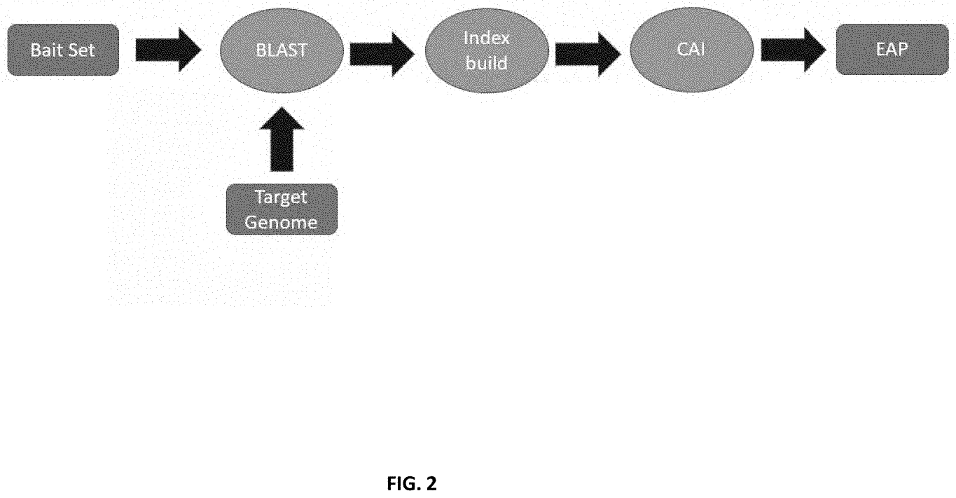

A computational pipeline was constructed which:
1) Starts with a Bait Set of highly expressed proteins, relevant to the class of organisms (e.g., bacteria, eukaryotes);
2) Uses the BLAST algorithm to find the equivalent genes in the new organism of interest; 3) Calculates a CAI index using the reference set from the organism;
4) Calculates the CAI of all remaining proteins in the organism; and
5) Calculates the adjusted totals and percentages of each amino acid in the organism (FIG. 2).

In addition, proteomics data may be used to derive protein copy number data for the organism. This data can be used to create weighting tables for organisms or classes of organisms to further improve the accuracy of expression calculations.

In this manner, an “expression-adjusted proteome” (EAP) is created that more accurately reflects the amino acid composition expected when the organism is grown during production. The EAP is then fed into the PDCAAS calculations in Example 2.

Organisms used in the above may be identified by a variety of means, including but not limited to 1) retrieval from NCBI or other public databanks, 2) identification by sequencing. Novel organisms may be identified and sequenced using a variety of technologies including Illumina, PacBio, and Nanopore sequencing. These sequence data can then be assembled using a variety of methods known in the art. Non-limiting examples of assembly programs useful for this work include CANU (Koren et al, Genome Research (2017) Vol 27:722-736), and Raven (Vaser & Sikic, Nature Computational Science (2021), Vol 1:332-336). Organisms may then be annotated using programs such as Prokka (Seeman, BioInformatics (2014), Vol 30(14):2068-2069) for bacterial annotation, Maker (Cantarel et al, Genome Research (2008), Vol 18:188-196) for eukaryotic annotation, or any other annotation programs including custom-designed pipelines. The resulting annotation provides the CDS (Coding DNA Sequence) sequences needed for translation.

Verification of Calculations Using E. coli

Utilizing the Biopython CodonAdaptationIndex module, the accuracy of the results were verified using a custom re-implementation of the Sharp and Li method in Python. The protein copy number was estimated as an exponential function of the CAI.

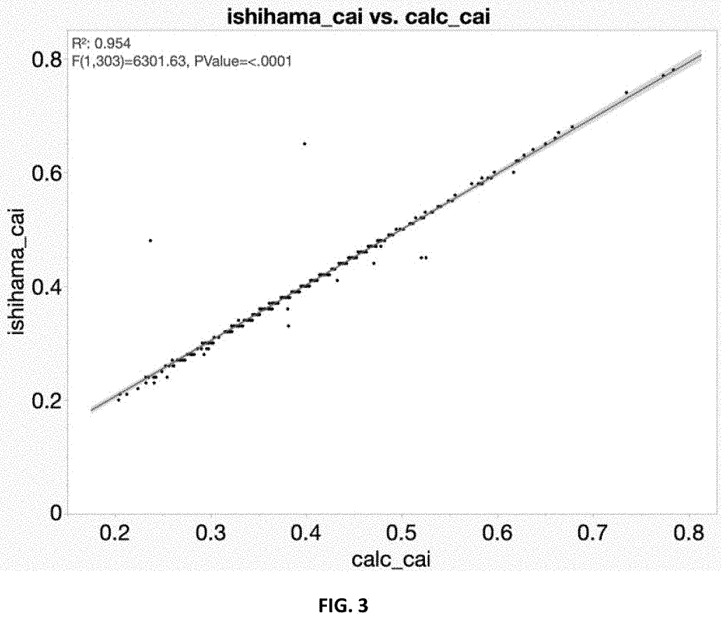

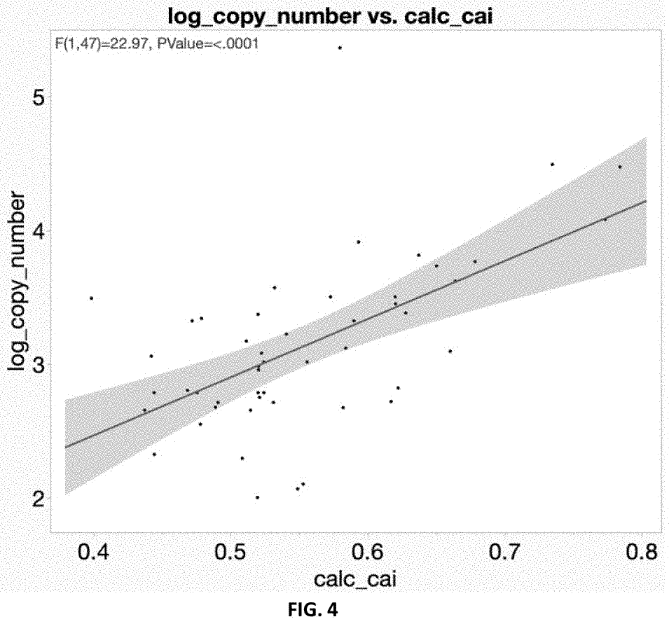

The accuracy of the CAI calculations was verified in the pipeline using E. coli (FIG. 3). The CAI calculations and protein copy number estimates matched well with the data from (Ishihama et al, BMC Genomics (2007), Vol 9(102):1-17). It was also generally observed that genes follow expected patterns, for instance, with ribosomal genes being called as highly expressed. FIG. 3 shows CAI calculations from the PDCAAS pipeline vs. Ishihama (R²=0.954, F-test p-value<0.0001). FIG. 4 shows a comparison of calculated CAI data with protein expression estimates (log copy number) from Ishihama (F-test p-value<0.0001). Similar to the Ishihama paper, a Spearman's rho correlation coefficient of 0.56 (p-value<0.0001) for this data was calculated. This showed that using an exponential function of CAI can give a good estimate of the true protein copy number in the cell.

### Extension to Other Organisms

Next, a pipeline was constructed to run additional organisms, such as yeast. A method was also developed to automatically find reference genes in a new organism. In this manner, any variety of organisms, such as non-conventional yeast, bacteria, or even plants can be run through the pipeline. The system is capable of downloading proteomes from public sources such as Uniprot, Genbank, or NCBI automatically for processing to create adjusted proteomes for a wide variety of species.

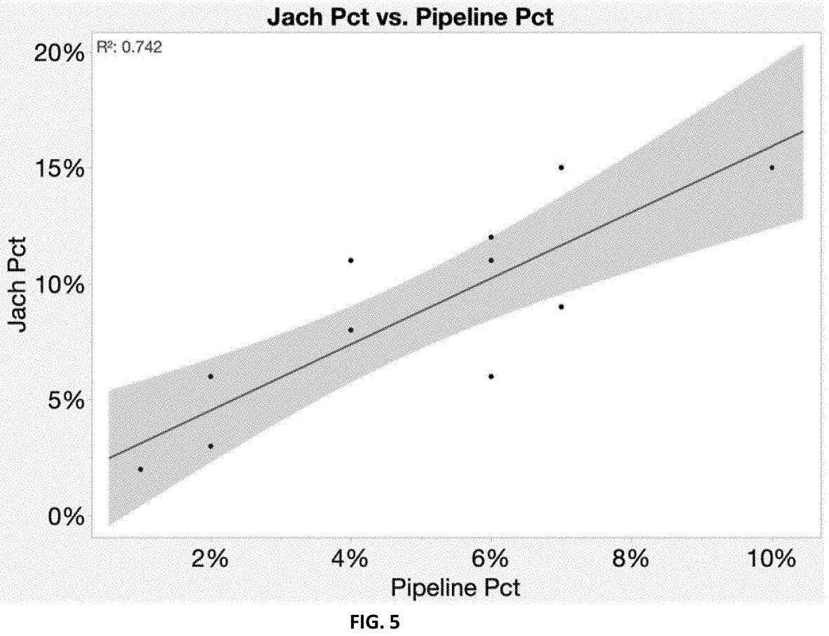

The resulting EAP and percentage amino acids were compared to amino acid percentages from dried Brewer's yeast (FIG. 5). FIG. 5 compares the amino acid percentages for Saccharomyces cerevisiae reported in (Jach et al, Metabolites, Vol 12(1):63) to those calculated by the pipeline (R²=0.742, F-test p-value=0.0002).

### Results and Conclusion

This example demonstrated an accurate correspondence between amino acid profiles calculated by the pipeline and those known in the literature. The resulting EAP data is useful for estimation and analysis of putative proteomes for nutritional value and for subsequent calculation of downstream properties, such as PDCAAS.

### Example 2: In Silico PDCAAS Calculation

### Purpose

The purpose of this example was to estimate protein quality using the EAP. PDCAAS (Protein Digestibility Corrected Amino Acid Score) is a method used widely in the industry to determine protein quality.

### Methods

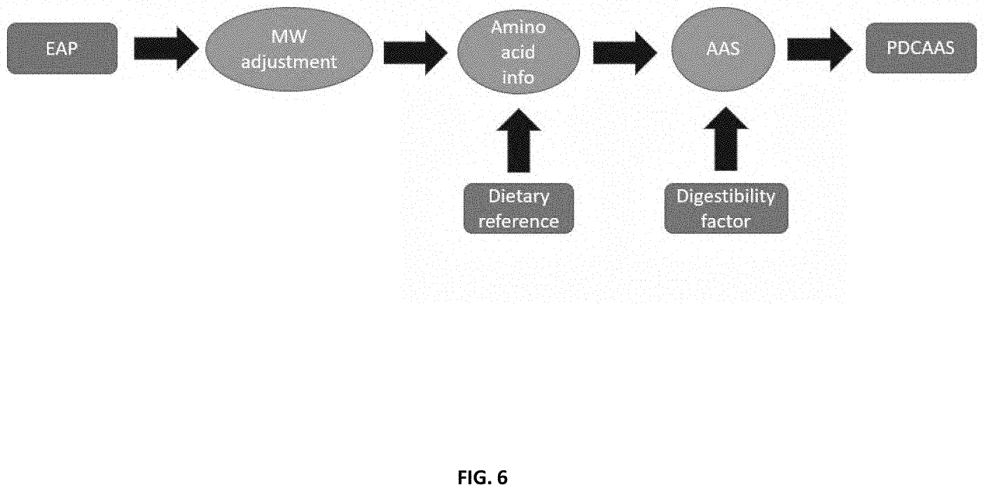

An algorithm was developed, which consists of:
1) A calculation of molecular-weight adjustments for EAP to obtain total amino acid content and percent by weight;
2) A comparison to a standard dietary reference;
3) A computation of AAS (Amino Acid Score) as compared to the reference;
4) An adjustment by digestibility factor; and
5) A PDCAAS calculation (FIG. 6).

The most limiting amino acid was scored compared to the dietary reference and stored as LRAAS (Least Represented Amino Acid Score).

From this, a PDCAAS score of 1.42 (typically rounded down to 1) was calculated for Saccharomyces cerevisiae. Both the unadjusted score (unadjusted PDCAAS or pdcaas_unadj) for ranking of organisms and the “rounded down” score were stored, which could have been reported. In this manner, organisms with a PDCAAS>1 can be ranked. An unadjusted PDCAAS of 1.43 for Lactobacillus sanfrancicensis (PDCAAS=1) was also calculated. In addition, the PDCAAS for >600 Gluten and Gliadin proteins from the GluPro-v1 database were calculated (GluPro v1.0 database: Gluten protein sequence database, The University of Manchester Library), known to typically have low PDCAAS (see: “PDCAAS—What's this all about”, Merieux NutriSciences). This analysis showed PDCAAS scores ranging from ˜0.15 to ˜0.3, consistent with expectations (see Example 15, “Use of the algorithm on individual proteins”).

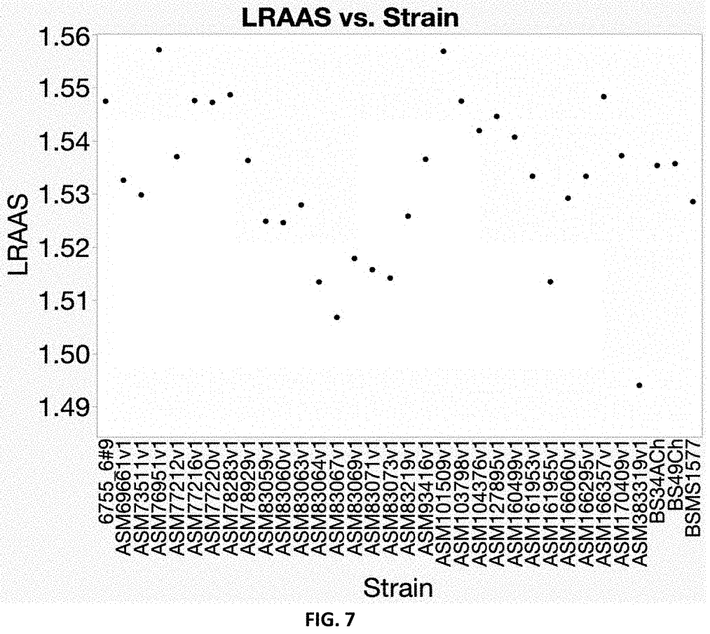

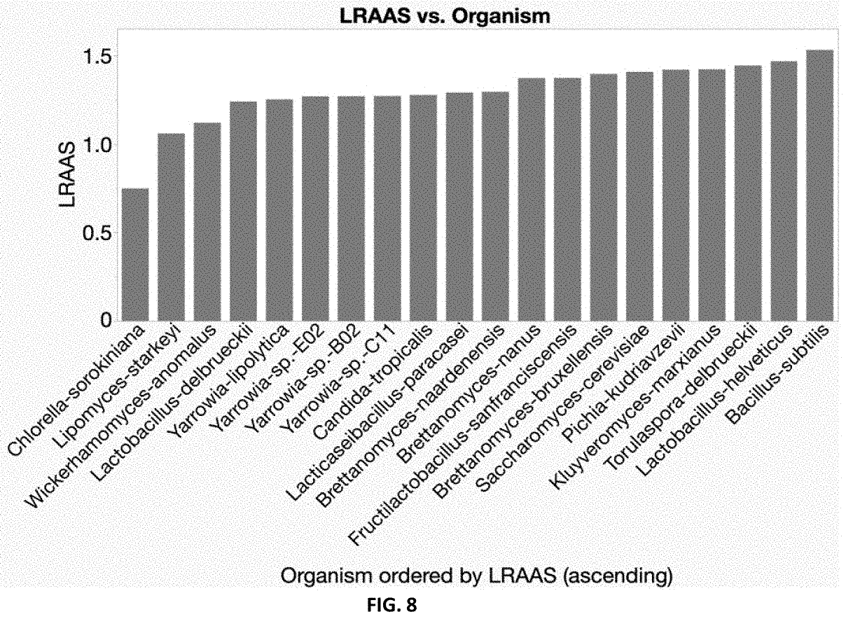

The PDCAAS scores for Bacillus species were further analyzed. These organisms were expected to have a high PDCAAS score and have been shown to upgrade the PDCAAS of Pea protein flour (Batbayar, Thesis, University of Saskatchewan (2022)). FIG. 7 shows the distribution of LRAAS scores for several strains of Bacillus. As expected, these organisms have high LRAAS scores, with unadjusted PDCAAS scores ranging from ˜1 to 1.26 (PDCAAS=1).

These scores allow for calculation of PDCAAS in silico for many organisms, ranging from bacteria to lower eukaryotes such as yeast, through higher plants. These scores allow for determination of ideal organisms or ideal mixtures of organisms used to create protein for optimal nutritional quality.

Characterization of Results from the Pipeline

To provide some initial characterization of results from the pipeline, the calculated PDCAAS scores with known PDCAAS data from the literature were compared. These results are presented in Table 1, showing good correspondence between predictions from the pipeline and literature.

TABLE 1
Comparison of PDCAAS pipeline results to Literature

| | PDCAAS | Expected | |
|---|---|---|---|
| Organism/Protein | Predicted | Value | Reference |
| Bovine Serum | 0.84 | ~1 | |
| Albumin | | | |
| Casein | 0.96 | ~1 | Hoffman & Falvo, Journal of |
| | | | Sports Science & Medicine |
| | | | (2004), Vol 3(3):118-130 |
| Saccharomyces | 1 | 0.9-1 | Zeng et al, Foods (2022), Vol |
| cerevisiae | | | 11(21):3326 |
| Chlorella | 0.64 | 0.64 | Wang et al, Foods (2020), Vol |
| sorokiniana | | | 9(11):1531 |

### Ranking of Organisms

The PDCAAS pipeline was utilized to make predictions for numerous yeast and bacteria which typically do not have published PDCAAS information. The data was obtained from NCBI datasets web site and run through the pipeline. The LRAAS score was used to help distinguish the strains. It was also possible to examine the amino acid which was least represented. In this manner, an organism may be selected with the maximum overall amino acid score and digestibility, and organisms can be selected whose least present amino acid may be complementary to another organism or protein product, such that they can be mixed to provide even higher levels of nutritional amino acid availability (see mixture algorithm, Example 10).

### Subsetting, Parallelization and Cloud Computing

The system presented herein can be run on a typical Linux-based computer, such as an Apple Macintosh computer, or can be run on any typical virtual machine running the Linux operating system. The system can be run on cloud computing systems employing these virtual machines, including but not limited to, Microsoft Azure, Amazon Web Services (AWS) or Google Cloud Platform (GCP).

To handle larger proteomes, subsets of proteomes can be used as a robust surrogate for PDCAAS calculations (see Example 14, “Algorithm performance on a Plant genome”). This can be performed using the make_subsets tool.

To handle larger proteomes, or larger sets of many proteomes, the system can be scaled via parallelization. In the simplest case, the “embarrassingly parallel” method can be used as follows: A given proteome can be split into multiple subsets; each subset can be run on a virtual machine using a Batch job launching and management system, such as AWS Batch or Microsoft Azure Batch; the results of the jobs can be collated for a final PDCAAS calculation which averages over the results from each job.

### Results and Conclusion

This example demonstrated an accurate correspondence between the final PDCAAS calculations and those known in the literature.

### Example 3: In Silico DIAAS Calculation

### Purpose

The purpose of this experiment was to determine if the method can estimate protein quality using the EAP from Example 1. DIAAS (Protein Digestibility Corrected Amino Acid Score) is a method used widely in the industry to determine protein quality.

### Methods

The DIAAS calculation provided a superior analysis compared to PDCAAS, which can generally underestimate the value of high-quality proteins and overestimate the value of low-quality proteins (Wolfe et al, Nutr. Rev. (2016), Vol 74(9):584-99). DIAAS takes into account the differing digestibility of different amino acids, and can obtain scores of >100, whereas the PDCAAS value is typically truncated at 1. These characteristics made the DIAAS a superior measure for identifying ultra-high quality protein sources.

The AAS Table, as described in Example 2, was used and appropriate reference sources to calculate an in silico DIAAS as follows:

DIAAS=100*lowest value of the digestible indispensable amino acid reference

For DIAAS scores, protein claims are as follows:
<75%, no protein quality claim
75-99%, good protein quality claim
≥100%, excellent or high protein quality claim

### Results and Conclusion

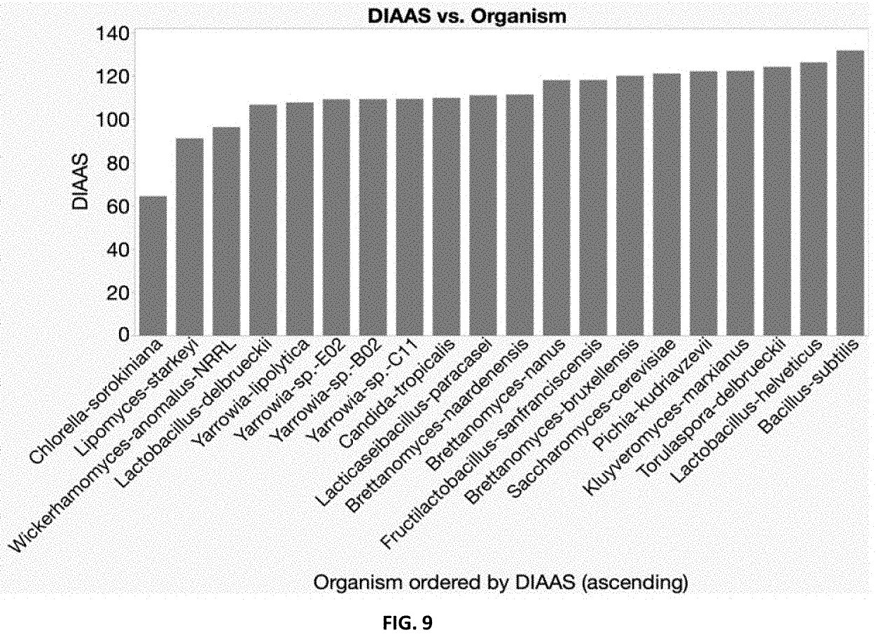

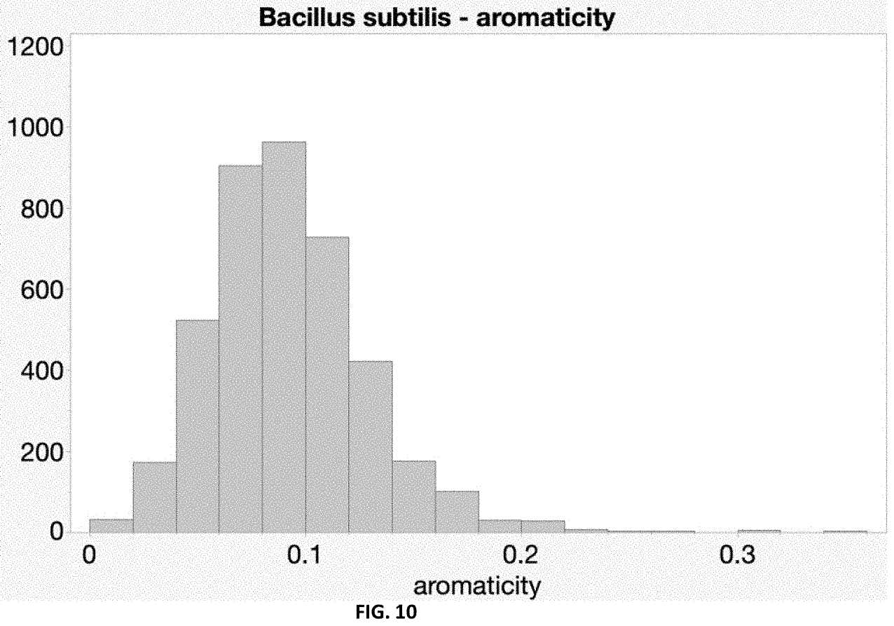

The results of this example demonstrated that the algorithm could scan several organisms, or mixtures thereof, to obtain extremely high quality DIAAS scores. DIAAS scores of >100 are desired. Based on laboratory data, scores of individual amino acids can be further adjusted based on their apparent digestibility. FIG. 9 shows an example of in silico DIAAS scores calculated for various organisms. High scores were generally obtained for key species of yeast or bacillus and a low score is seen for Chlorella, as expected.

### Example 4: Physicochemical Properties of Proteins

### Purpose

The purpose of this example was to determine if the algorithm of Example 1 can report other key characteristics of the EAP, including nitrogen content, branched-chain amino acids (BCAA) etc. These characteristics could then be used to determine ideal protein nutritional quality, as well as be used as inputs for machine learning algorithms to improve components of the algorithm.

### Methods

The algorithm from Example 1 also provided a comprehensive report for the analysis of the organism's proteome. The system also has the ability to produce a variety of graphs comparing multiple organisms supplied in a batch mode. The system was capable of providing both averaged values of various calculations over the EAP, and also full distributions of values for all proteins in the proteome. Therefore, the system also searched proteomes for individual proteins of value. The ProtParam module (See web.expasy.org/protparam/) was used to calculate a variety of parameters on individual proteins including Gravy score, Isoelectric point, etc. ProtLearn (See github.com/tadorfer/protlearn) is an alternative package for determining protein properties, similar to ProtParam.

Taken together, the expression adjustments, AAS scores, ProtParam data, and other values reported by the system comprised an Adjusted Proteome DataBank (APDB) which forms the basis for all downstream algorithms. It provided a platform upon which machine learning can be performed to derive further analyses described in subsequent examples. As mentioned previously, the system can draw from a wide variety of public data sources, as well as store custom in-house sequenced & assembled genomes/proteomes, thus allowing for the storage of a large number of expression-adjusted and AAS-calculated proteomes within the APDB.

FIG. 10 shows the aromaticity distribution for all proteins in one organism (Lobry et al, Nucleic Acids Research (1994), Vol 22:3174-3180) using the ProtParam module implemented above. Aromaticity can be used to understand various protein properties such as aggregation, which is important in food ingredients (See Tartaglia et al, Protein Science (2004), Vol 13(7):1939-1941).

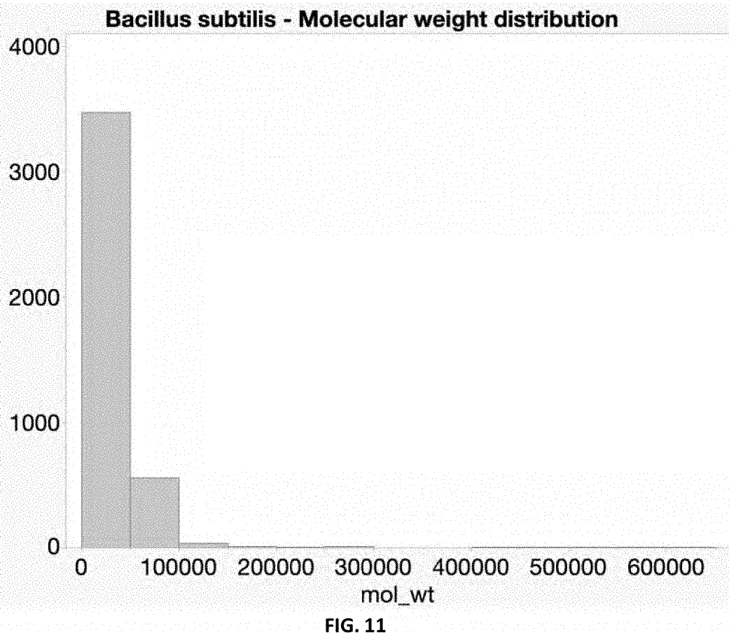

FIG. 11 shows the distribution of molecular weights for all proteins in one organism (Bacillus subtilis) using the ProtParam module implemented above. In general organisms with proteomes shifted to higher molecular weights will show less digestibility. Fermentation of cereal grains, and processing of food products, can have impacts on particle size and molecular weight, and hence digestibility and texture, of food products (Li et al, Food Research International (2017), Vol 92:88-94) (Chima et al, Foods (2022), Vol 11, 2846).

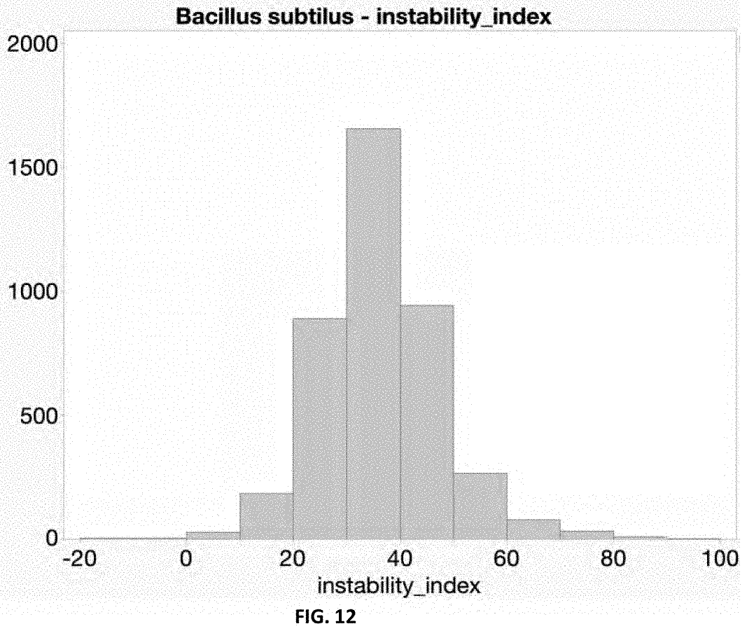

FIG. 12 shows the instability index (Guruprasad et al, Protein Engineering Design & Selection (1990), Vol 4(2):155-161) for one organism, Bacillus subtilis, using the ProtParam module implemented above. This method uses the occurrence pattern of dipeptides to predict stability of proteins. Protein instability can be used to predict emulsion stability (Elizalde et al, Journal of Food Science (1991), Vol 56(1):116-120) and shelf stability (Barnett & Kim, Food Storage Stability (1997), Ch. 3:75-87) of foods.

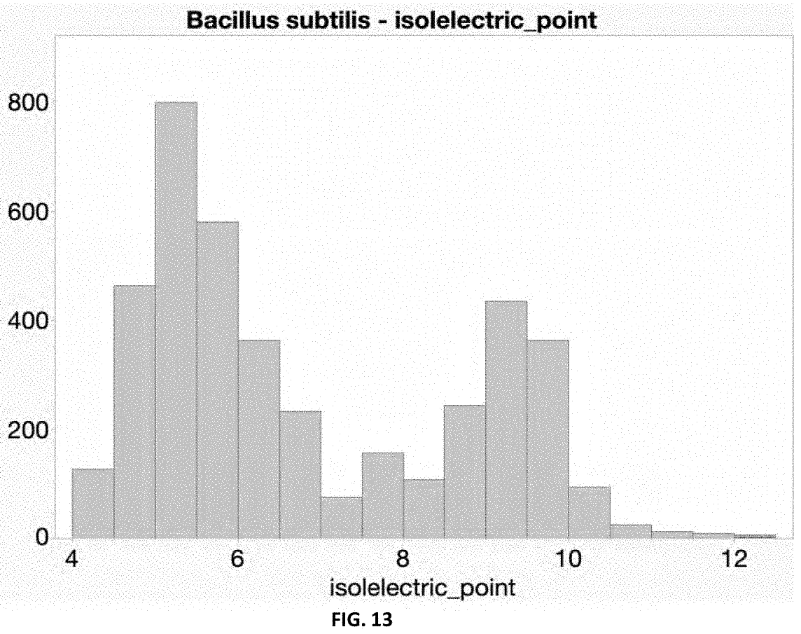

FIG. 13 shows the isoelectric point distribution for one organism, Bacillus subtilis, using the ProtParam module implemented above. Isolelectric point of proteins can influence digestibility and the formation of starch-protein-lipid complexes (Lin et al, LWT (2020), Vol 134).

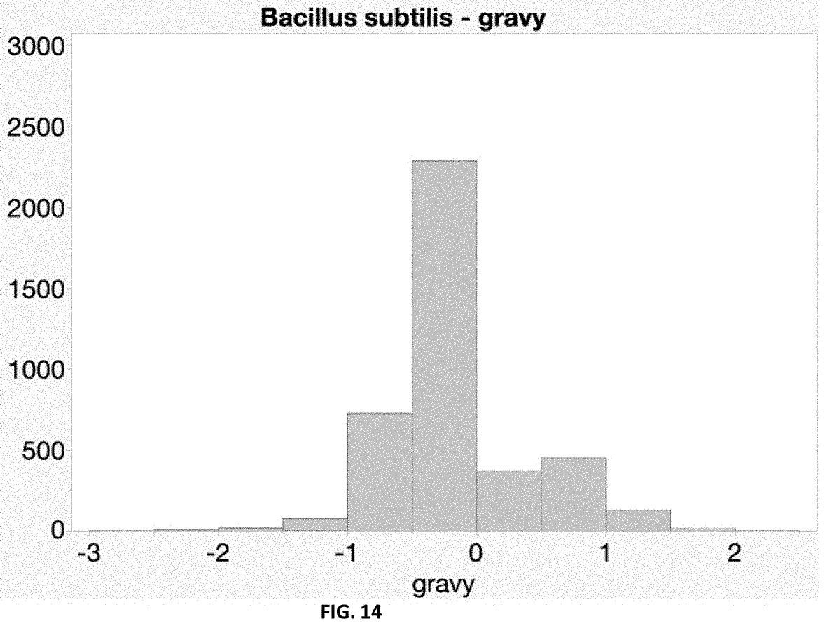

FIG. 14 shows the Gravy score distribution for one organism, Bacillus subtilis, using the ProtParam module implemented above. The gravy score follows the method of Kyte and Doolittle and indicates the hydrophobicity of proteins. Hydrophobicity plays important roles in food structure, emulsifying capacity, stability, and fat binding capacity (Nakai, Journal of Agricultural and Food Chemistry (1983), Vol 31(4):676-683) and foaming characteristics (Townsend & Nakai, Journal of Food Science (1983), Vol 48(2):588-594).

The Nitrogen content of a proteome can also be calculated using a table of Nitrogen contents for amino acids by mass (See scienceofagriculture.org/nitrogen/protein/averages.html).

The BCAA content of a proteome can be calculated by totaling the levels of branched-chain amino acids, Leucine, Isoleucine, and Valine.

The properties of this example can be correlated to protein solubility, an important component of food properties when using alternative protein (See Grossman & McClements, Food Hydrocolloids (2023), Vol 137, 108416). This knowledge can then guide food processing methods to maximize proper mixing of protein ingredients and improve texture and protein delivery in foods.

### Results and Conclusion

The results of this example demonstrated that the protein properties can be used as additional inputs to machine learning algorithms to enhance the accuracy of various components in the algorithm and to provide further selection criteria for organisms in functional food ingredients.

### Example 5: Calculation of Digestibility Factors

### Purpose

The purpose of this example was to determine if the PDCAAS system from Example 1 can calculate digestibility factors.

### Methods

Digestibility factors were supplied via a table using values from the literature (table: digest_ref). For example, the digestibility of mycoprotein, a commonly used fungal protein, is given as 0.86 (Edwards & Cummings, Proceedings of the Nutrition Society (2010), Vol. 69). Digestibility factors for microalgae may vary anywhere from 47-57% (Janczyk et al, Animal Feed Science & Technology (2007), Vol 132(1-2):163-169). Some organisms, such as microalgae, possess an especially rigid cell wall which affects digestibility, and this can also be accounted for in the digestibility factor. Digestibility for yeast is given as 85-86% in (Mateo & Stein, Canadian Journal of Animal Science (2007), Vol 87(3):381-383).

Digestibility factors also varied based on processing techniques used, organism, and type of study. For example, spray drying and steam explosion can enhance digestibility. Digestibility factors can also be supplied via lab-derived values based on in vitro digestibility studies.

Digestibility factors were also estimated using machine learning algorithms which examine the properties of proteomes. One key factor for proteome digestibility is the number of beta sheets and alpha helices present (Yu, British Journal of Nutrition (2005), Vol 94:655-665) (Zhao et al, Italian Journal of Animal Science (2022), Vol 21(1):507-521). High levels of beta sheets are associated with low digestibility (Carbonaro et al, Amino Acids (2012), Vol 43:911-921). Digestibility is positively correlated with number of alpha helices and the alpha helix/beta-sheet ratio (ABR) (Bal et al, Asian-Australas Journal of Animal Science (2016), Vol 29(8):1159-1165).

Next, the algorithm outlined in Example 1 was used to predict digestibility factors for whole microbial proteomes. Bi-directional Long Short Term Memories (biLSTM) are machine learning architectures useful for analyzing data such as words, sentences, or protein grammars (See baeldung.com/cs/bidirectional-vs-unidirectional-lstm). In this method, a recurrent neural network is constructed with gates that help control how much input is used at each step and how much input is forgotten, improving issues with exploding gradients in training. In addition, the system learns from both previous and upcoming sequence information, which improves contextual understanding. PARROT is a framework for implementing biLSTMs (Griffith & Holehouse, ELife (2021), Vol 10:e70576).

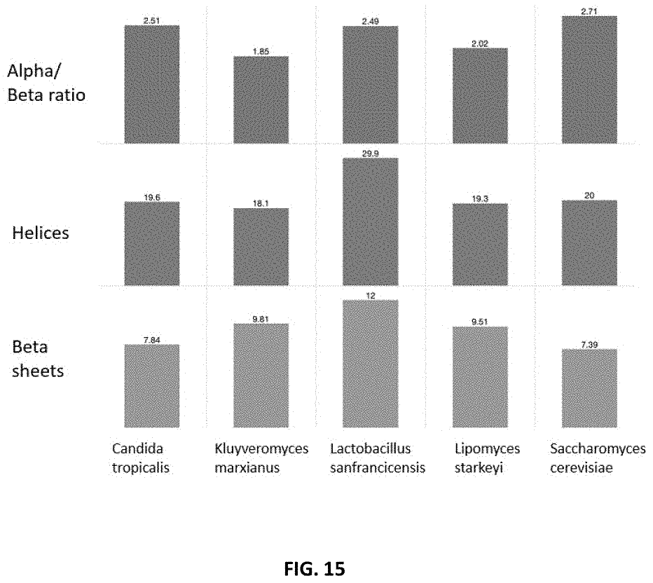

A machine learning model (biLSTM) was trained using PARROT to recognize alpha helix and beta sheet structures in proteins. The system was trained using a subset of results from proteomes using JPRED (Drozdetskiy et al, Nucleic Acids Research (2015), Vol 43(W1): W389-394) a protein secondary structure prediction tool. First, a subset of proteomes was examined using JPred to predict alpha helix and beta sheet structures (FIG. 15). This shows that variation in total number of alpha helices, beta sheets, and ABR can be detected.

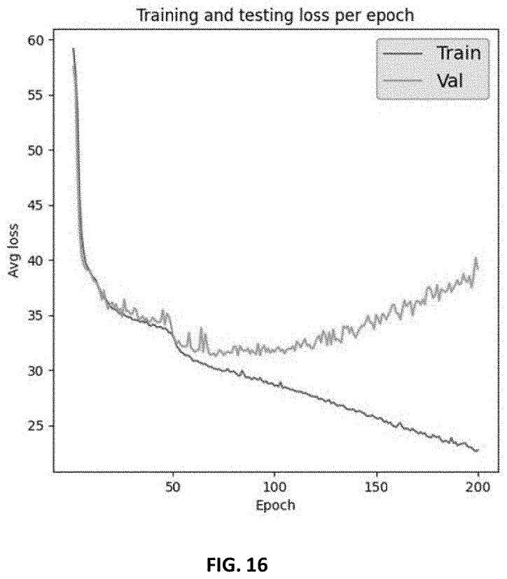

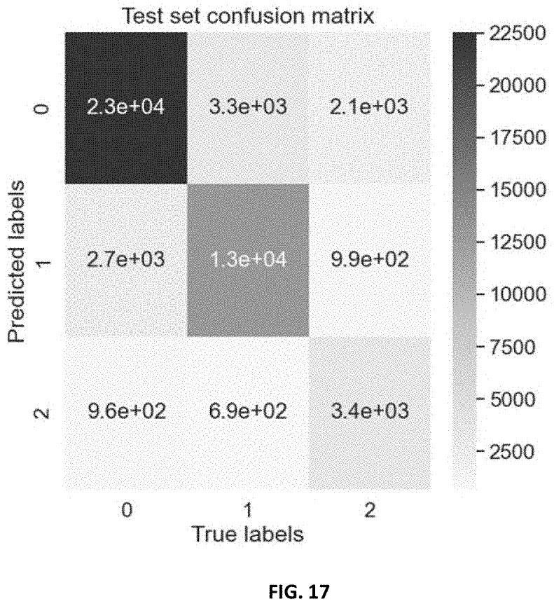

Next, a biLSTM was trained using PARROT and made predictions on a larger dataset consisting of entire proteomes. FIG. 16 and FIG. 17 show the results of the training including the training loss and confusion matrix, respectively. The system was built using 10 layers and run for 200 training epochs. The resulting model obtains an accuracy of 78%, F1 score of 0.78, and MCC of 0.624 (Table 2). The F1 score is the harmonic mean of the precision and recall for a test as described in the art, which a measure of the test's accuracy in a binary classification (See en.wikipedia.org/wiki/F-score). The MCC is the Matthews Correlation Coefficient of the true and predicted values (Seethedatascientist.com/metrics-matthews-correlation-coefficient/). These values show a good overall correlation of the model with expected results

TABLE 2
Result metrics for biLSTM training

| | |
|---|---|
| Accuracy | 78% |
| F1 Score | 0.78 |
| MCC | 0.625 |

Next the machine learning model was used to make predictions on proteomes of various organisms. These predictions can then be used to estimate digestibility coefficients. For example, Saccharomyces cerevisiae has an ABR of 0.275, compared to Lipomyces starkeyi at 0.226. This allows for a comparison of the comparative ratio of ABRs, and for calculation of an adjusted digestibility coefficient for Lipomyces starkeyi, based on a baseline of 86% digestibility for yeast, as follows:

Adjusted Digestibility=0.86*(0.226/0.275)=70.52%

Other approaches to modeling protein secondary structure in addition to LSTMs can be taken. These include the use of full 3-D prediction algorithms such as AlphaFold, CNNs, and other types of networks. A review of these approaches is provided in (Ismi et al, Computational and Structural Biotechnology Journal (2022), Vol 20:6271-6286).

Large Language Models (LLMs) are statistical distributions over word frequencies. They have been applied extensively to natural language processing and are now being applied successfully to protein sequences. These models are trained on large numbers of protein sequences in public databases. Examples include ProtGPT2 (Ferruz et al, Nature Communications (2022), Vol 13(4348)) and ProGen (Madani et al, Nature Biotechnology (2023)).

Language models have been used to model protein secondary structure (Heizinger et al, BMC BioInformatics (2019), Vol 20:723).

Transformers (Chandra et al, ELife (2023) Vol 12:e82819) are models which encode data into internal representations and then recode them for output. Transformers have been used to predict numerous protein properties. For example, AttSec (Kim & Kwon, Research Square, Preprint) has been used to predict protein secondary structure.

An LSTM, LLM, Transformer, or similar model could be used to directly fit a proteome sequence to a set of observed digestibility values, bypassing calculation of secondary structure.

### Results and Conclusion

The results of this example demonstrated that the method can be used to create new PDCAAS values for organisms using the more refined method for estimating digestibility. For example, by applying the Adjusted Digestibility calculated using machine learning on secondary structure above, the PDCAAS score for Lipomyces starkeyi changes from 0.97 to 0.8 using this method. Furthermore, the results of this example indicate this method can be used to create more refined estimates of PDCAAS for organisms using their proteomes, by making in silico estimates of digestibility, and is hence applicable even to organisms for which digestibility information is not available.

### Example 6: PDCAAS & Euclidean Distance Analysis

### Purpose

The purpose of this example was to determine if the PDCAAS system from Example 1 can also be used to compare the entire amino acid profile of the proteins of an organism, after expression adjustment, to the amino acid profiles of common foods such as eggs, chicken, milk, etc.

### Methods

The system can also be used to compare the entire amino acid profile of an organism, after expression adjustment, to the amino acid profiles of common foods such as eggs, chicken, milk, etc. This allowed for the selection of microbial organisms for protein ingredients most closely matching certain desired profiles.

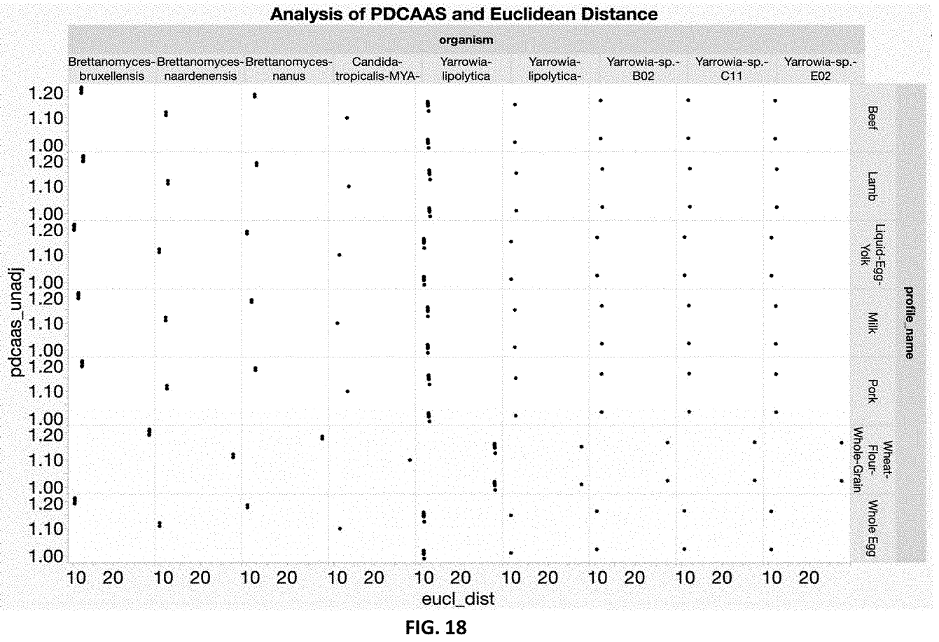

FIG. 18 shows the Euclidean distance of various microorganisms compared to different amino acid profiles on the X axis. The unadjusted PDCAAS is shown on the Y axis. The organisms selected by the algorithm were far away from Wheat, a low nutritional source. For example, they were much closer to either beef, lamb, whole egg, or milk, which are excellent protein sources containing a balanced amino acid profile. As one example, Brettanomyces bruxellensis had a high PDCAAS and is very close to liquid egg yolk. In another example, a cluster of Candidas can be seen which have a high PDCAAS and are close to Milk profile.

### Results and Conclusion

The results of this example demonstrated that the method identified organisms with amino acid profiles similar to beef, lamb, whole egg, and milk.

### Example 7: Fermentation of Protein Product & Measurement of Amino Acid Profiles (AAP)

### Purpose

The purpose of this example is to grow microorganisms with a desired PDCAAS, physiochemical properties, and/or digestibility properties, produce a protein powder, and to determine the AAP.

The samples may be typically prepared by a Shake Flask fermentation method (See Example 8, “Measurement of IVPDCAAS for an Organism”, or a BioReactor fermentation method).

### Methods—BioReactor Fermentation

Samples are prepared from a fermentation broth by growing the microorganism on a carbon source such as dextrose, sucrose or complex starches like grain meals, flour, malted barley etc. The fermentation broth comprises of other nutrients like a nitrogen source, phosphate source, and minerals such as sodium, potassium, magnesium, iron, calcium, manganese, and other trace elements, like cobalt, nickel, selenium, etc. and micronutrients like thiamine, biotin, riboflavin etc., as required for the growth of the microorganism. The microorganism media can also be derived from complex nutritional sources such as peptone, yeast extract, corn steep liquor, extracts of other industrial products etc. Fermentation is carried out at a temperature, pressure and pH that allows for maximum microbial growth. The amount of oxygen is regulated by air flow, reactor headspace, and agitation and is contingent on the organism's tolerance to oxygen concentrations and shear rates. Fermentations are carried out in a bioreactor system until the desired biomass is obtained. At the end of fermentation, the biomass is washed to remove impurities and salts. Biomass is further purified to remove cell wall, DNA and RNA as well as excess water, and subsequently dried to obtain a powder product that is enriched in protein (Yuan et al, Ultrasonics Sonochemistry (2022), Vol 86:106012).

### Methods—Amino Acid Profiling

Amino acid profiles are measured using standard methods. Typically, amino acid composition is measured by AOAC 994.12. Standard vendors include Aemtek, and Medallion Labs. Briefly, following HCl acid hydrolysis, there is performic acid oxidation for methionine and cysteine analysis, and base hydrolysis for tryptophan analysis, with ion-exchange chromatography followed by post-column derivatization with ninhydrin and detection by ultraviolet-visible spectrophotometry, as described in, (“Amino Acid Analysis Protocols”, Packer & Williams, Vol 159, Methods in Molecular Biology, Humana Press).

Amino acid profile results provide information on total protein, total nitrogen content, and percent of amino acids present in the sample. The measurement of total protein and nitrogen is performed using the Dumas method preferably, or alternatively the Kjeldahl method. Dry matter content and total moisture content are determined by oven drying at 180° C. for 1 hr or through an instrument, like moisture analyzer that measures the rate of change in mass upon drying.

### Results and Conclusion

The results of this experiment will demonstrate that organisms selected for with a desired PDCAAS, physiochemical properties, and/or digestibility properties can be fermented to produce a protein powder. The measured AAP of these protein powders are expected to correspond to the predicted AAP.

### Example 8: Measurement of IVPDCAAS for an Organism

### Purpose

The purpose of this experiment is to determine if the method can estimate in vitro PDCAAS (IVPDCAAS).

### Methods

The IVPDCAAS is measured for organisms using standard methods such as those offered by Medallion labs.

### Method—Shake Flask Fermentation

Aseptic shake flask fermentations were performed for production of biomass for PDCAAS analysis using a working media of Yeast Extract, Malt Extract, Peptone, and Dextrose at pH 6.2. The yeast strain of interest was revived in 250 mL baffled DeLong flasks from frozen glycerol stocks in the same working media at 50 mL working volume and allowed to grow overnight at 30° C. and 120 rpm. 4 L baffled flasks were used at 2 L working volume and inoculated with the overnight cultures at 10% v/v inoculation ratio. These were left to shake at 30° C. and 250 rpm and samples were taken periodically to monitor glucose concentration via YSI and biomass concentration via OD600. Fermentation concluded when glucose concentration was zero and biomass had reached a constant value.

Flasks were harvested by combining fermentation media into 1 L centrifuge containers which were centrifuged at 2000 rpm for 5 minutes. The supernatant was discarded, and the cell pellet was resuspended in DI water and centrifuged at 2000 rpm for 5 minutes. This step was repeated with a final centrifugation performed at 5000 rpm for 10 minutes. The supernatant was discarded and the cell pellets were combined, frozen at −80° C., stored for 1 week, and sent on dry ice using overnight shipping to Medallion Labs for PDCAAS analysis.

### Method—In Vitro Digestion & IVPDCAAS Analysis

Samples as prepared above are put through a Human digestion simulation (in vitro PDCAAS analysis) as follows:
1) Breakdown of proteins into amino acids;
2) Reaction with Ninhydrin; and
3) Digestibility determination.

This report then provides several pieces of data including: PDCAAS, amino acid profile, sample moisture, and total protein.

This approach can also be used to compare IVDIAAS results. IVDIAAS is measured by the INFOGEST method. Briefly, protein contents are put through a series of simulated digestion steps using predefined reagents, and protein and amino acid contents are measured before and after digestion. A protein conversion is performed based on Nitrogen content along with Degree of Hydrolysis analysis, followed by True Ileal Digestibility calculation. Finally, this is used to compare to a reference standard, resulting in an IVDIAAS calculation.

To demonstrate the use of the algorithm in Yeast, the method was used on several other yeast species, in addition to Saccharomyces cerevisiae. An organism predicted to be high in PDCAAS (Kluyveromyces marxianus) was selected for Shake Flask Fermentation & PDCAAS analysis.

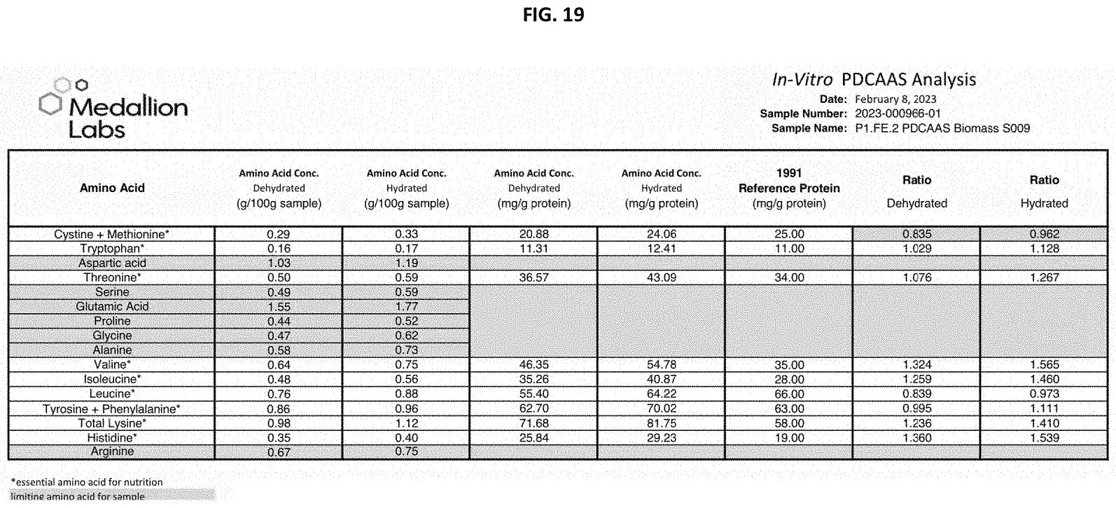

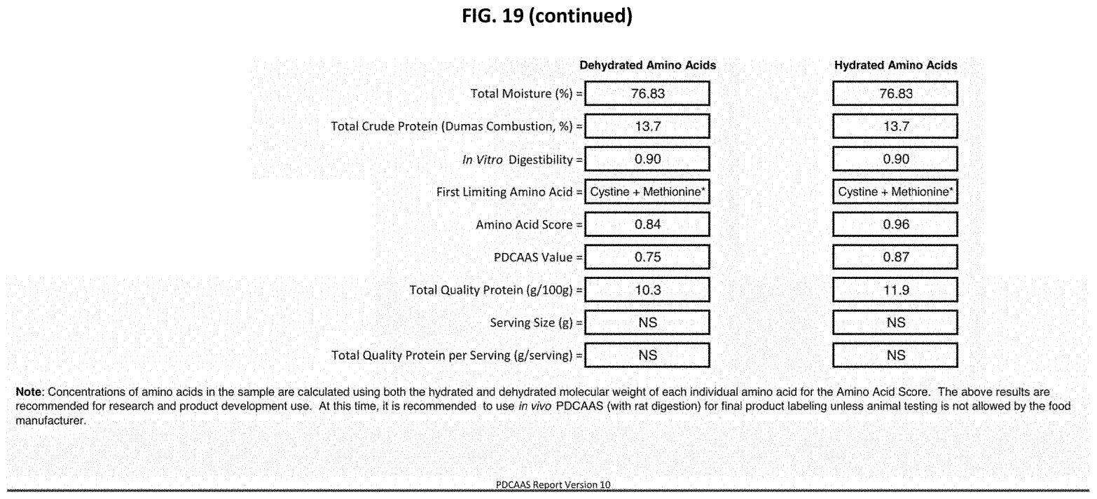

The organism Kluyveromyces marxianus CBS397 was submitted for PDCAAS analysis using Medallion Labs in vitro PDCAAS analysis (Sample 2023-000966-01 P1.FE.2 PDCAAS Biomass 5009 Feb. 8, 2023, Report #70361). FIG. 19 shows the results of the PDCAAS analysis.

The results of the analysis show the most limiting amino acid as M+C, which is correctly predicted by the pipeline.

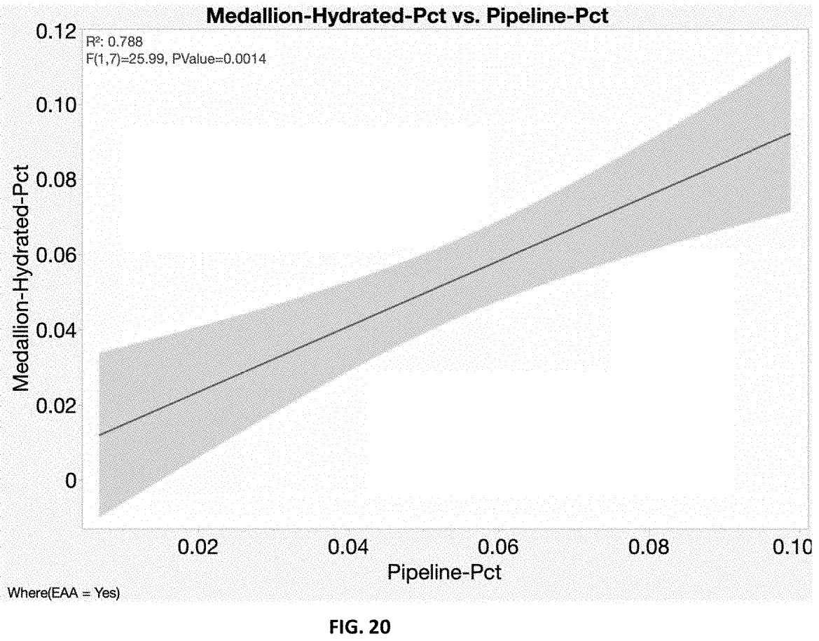

The amino acid analysis comparison is shown in FIG. 20. The comparison of EAA for the pipeline prediction vs. relative percentage of amino acids reported by Medallion gives and R2 of 0.788 and significant F-test of p<0.0014.

The PDCAAS from the pipeline, when adjusted by the PAL (see Example 9, “Process Algorithm Layer”) value using “low” processing as performed here, is 1.22*0.7=0.85. This is quite close to the value reported by Medallion of PDCAAS=0.87.

### Results and Conclusion

This example demonstrates the method provides an accurate correspondence between the ISPDCAAS calculations and the laboratory measured IVPDCAAS.

### Example 9: Process Algorithm Layer—Learning of Relationships for in Silico and In Vitro PDCAAS and DIAAS to Capture Downstream Processing & Other Properties

### Purpose

The purpose of this experiment is to determine if the algorithm from Example 1 can adjust in silico measurements to predict an even higher accuracy of protein nutritional quality.

### Methods

Based on the predicted ISPDCAAS (or ISDIAAS) and in vitro data, a relationship can be learned to adjust the in silico measurements to be of even higher accuracy. This helps consider the variety of alterations that can occur to the proteome during processing of the sample.

Sources of potential error in the algorithm include the expression adjustment, which may fail to take into account the complexities of biology in varying organisms or scaling factors in protein copy number related to codon usage. These differences can be approached by creating custom adjustment factors for different organisms or by choice of scaling function (e.g. exponential function).

Another source of potential error could come from the use of downstream techniques to dry, ground, process, store, or treat the protein material. In addition, measurements taken from further downstream products, such as for example baked goods derived from the addition of protein powder, may introduce further changes during the baking process.

Fermentation methods and parameters used can alter PDCAAS (Batbayar, Thesis, 2022).

Downstream Processing (DSP) can affect digestibility and other food ingredient characteristics in multiple ways (Gu et al, Food Reviews International (2022)).

As such, the concept of a Process Algorithm Layer (PAL) is introduced. This module adjusts PDCAAS and other derived scores based on alterations in fermentation conditions, downstream processing, formulation, storage or other aspects of food production and delivery.

An algorithmic layer using the APDB from previous examples to predict in vitro AAS, PDCAAS, and DIAAS scores can be developed. Note that AAS is the most directly relevant information here, as PDCAAS and DIAAS simply involve multiplication by a standardized digestibility factor and comparison to a reference, and PDCAAS is focused only on the most limiting amino acid. Thus, since maximizing the value of the full overall AAS composition in protein is desired, optimizing the accuracy of the algorithm to predict the AAS table is focused on.

These adjustments can be approached using a variety of bioinformatics and machine learning methods. The choice of index set for CAI calculations can be adjusted, as can the method of CAI calculation and the function for protein copy number determination. One can also use machine learning algorithms to correct for scores based on class of organism and types of downstream processing.

In vitro data, including PDCAAS and Amino acid profile, can be fed to a machine learning algorithm. The algorithm may adjust various parameters including the CAI weighting algorithm used, type of exponential function, etc. The algorithm seeks to optimize the match between in silico predictions and in vitro data.

### Parameters Available for Use in Learning Layer:

Reference gene index set
CAI method (e.g. Sharp & Li, delta, Karlin, Machine Learning)
Protein copy number function (e.g. exponential)
Class of organism
APDB information
Digestibility factors
Downstream processing methods including drying time, heating, etc.
   Proteolysis and enzymes used
Baking methods used include heat, time, etc.
   Additional ingredients

An algorithmic layer, Process Algorithm Layer (PAL), is introduced that incorporates inputs for downstream processing and baking processes and mixing. This allows for prediction of protein quality properties at the end of a process. By using the many pieces of information in the APDB, the algorithm may more accurately predict final AAS scores. For example, the average and standard deviation of protein molecular weights in an organism might help better predict its final AAS distribution after proteolysis treatment and downstream processing.

In a typical example, an Artificial Neural Network (ANN) is trained to predict final scores for AAS based on inputs including the APDB and downstream process parameters.

As a simple non-limiting example three levels of processing are investigated, termed as “high”, “medium”, and “low”, in decreasing order of the level of downstream processing performed on the food ingredient. These were then assigned relative processing factors of 0.9, 0.8, and 0.7 respectively, reflecting the effect on digestibility of different processing methods.

More complex versions of the PAL may use machine learning approaches. For example, a linear regression could be performed to match predicted digestibility values with observed values. Alternatively, a neural network could be used to match predicted digestibility values to observed values. A language model or biLSTM may be trained to use the organism proteome and associated processing parameters to produce predicted digestibility values with improved match to observed values.

Additional machine learning methods for correlation are also listed here and can easily be applied by one skilled in the art to provide alternatives for the PAL.

### Alternative Machine Learning Methods for PAL:

Linear regression
Polynomial regression
Decision tree
Random forest
ANN (Artificial Neural Network)
CNN (Convolutional Neural Network)
Language Model
biLSTM

Note that when mixtures of protein products and other ingredients are used in a process, it is straightforward to calculate the final expected protein content, AAS, PDCAAS, and IVDIAAS of the final product by using a weighted, digestibility-adjusted formula combining the mixed ingredients (see Example 10, “Mixture prediction algorithm”).

Dimensionality reduction algorithms can also be used in this context to find the most relevant parameters from the APDB which predict protein quality in downstream products.

In addition to predicting properties after downstream processing, this algorithm layer can also be used to predict final food properties after baking.

A machine learning approach is taken to predict DIAAS in food products (Malvar et al, Springer Nature (2021), arXiv:2211.00625). This system takes a variety of information on Human foods such as fiber content, protein levels, and amino acid contents, and attempts to make predictions of DIAAS values from this using a variety of machine learning methods. The system does not make predictions from first principles using genomes and is focused on currently known Human foods. It does not make predictions about new microbial protein ingredients.

The PAL layer described in this example will be provided with additional information such as fermentation growth conditions, fermentation parameters, downstream processing methods, protein values from physicochemical calculations, fiber content, biomass composition, baking times and parameters, additional ingredients added to the food product recipe, food recipe parameters, and any other information as desired.

This will enable the PAL layer to make estimations of the amino acid content and PDCAAS (or DIAAS) of the microbial protein ingredient, and of the final, cooked food product, based on analysis of a genome or proteome.

### Results and Conclusion

The results of this example will demonstrate that the algorithm from Example 1 can adjust in silico measurements to predict an even higher accuracy of protein nutritional quality.

### Example 10: Mixture Prediction Algorithm

### Purpose

The purpose of this experiment was to determine if the algorithm from Example 1 can compute and store the PDCAAS scores from multiple organisms in a database and allow for the selection of ideal mixtures of protein.

### Methods

A given organism was selected that is deficient in one amino acid and can be adjusted by mixture. For example, Organism A may be deficient in amino acid A. Organism B has a surplus of amino acid A. The algorithm recommends a mixture of X % of Organism A and Y % of Organism B, resulting in a particular final predicted AAS table.

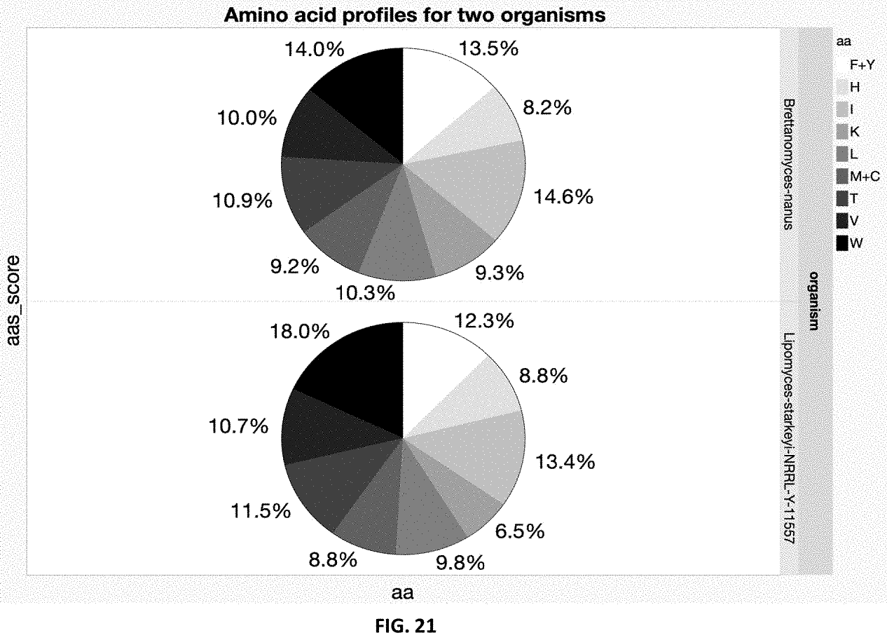

As an illustration of this approach, consider Brettanomyces nasus (Accession Number: GCA_011074865.2) and Lipomyces starkeyi (Accession Number: GCA_001661325.1). The algorithm calculated that Brettanomyces has a limiting amino acid of Histidine (H) and an unadjusted PDCAAS of 1.19. Lipomyces has a limiting amino acid of Lysine (K) with a higher level of Histidine, and an unadjusted PDCAAS of 0.97. FIG. 21 shows the profiles of the two organisms in terms of percentage of their respective EAA.

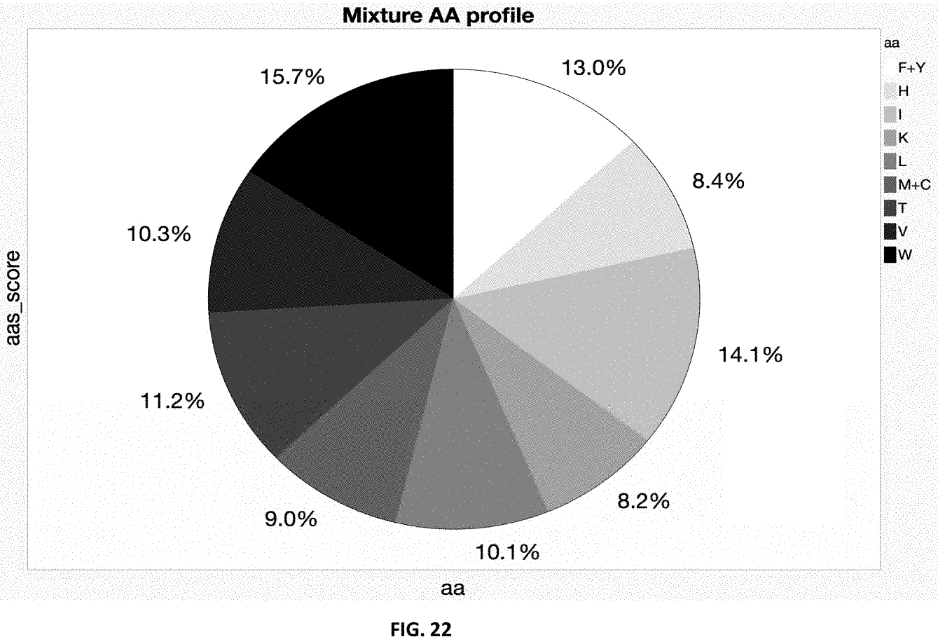

The mixture algorithm (tool: make_mixture) was next run and calculated a mixture of protein comprising 60% Brettanomyces protein powder and 40% Lipomyces protein powder. This resulting profile is shown in FIG. 22. This mixture increased the Histidine and Lysine contents with only minor changes to other more predominant amino acid categories, thus providing a more balanced amino acid and nutritional profile. In addition, this mixture was predicted to provide an overall PDCAAS of 1 (unadjusted PDCAAS=0.6*1.19+0.4*0.97=1.1).

### Results and Conclusion

The results of this example demonstrated that the mixture algorithm can be used to predict optimal mixtures of microbial protein ingredients to refine and optimize overall nutrition. In addition, results show this algorithm can be used to estimate the upgrading properties expected from microbial fermentations, in which a lower nutrition food source, such as grain or food waste, is nutritionally upgraded in a fermentation process, in which the microbe contributes a higher overall PDCAAS and superior amino acid profile, creating a higher nutrition ingredient.

### Example 11: Algorithm Performance on Additional Yeast

### Purpose

The purpose of this experiment is to use the method to identify species of yeast, and growth conditions for the yeast, with a desired PDCAAS, physiochemical properties, and/or digestibility properties.

### Methods

To demonstrate the use of the algorithm in Yeast, the method was used on several other yeast species, in addition to Saccharomyces cerevisiae. An organism predicted to be high in PDCAAS (Kluyveromyces marxianus) was selected for PDCAAS analysis (see Example 8, “Measurement of IVPDCAAS for an Organism”) giving expected results.

As a next step, several additional yeast will be selected and tested for IVPDCAAS, AAP, and comparison to pipeline results.

This data will be combined with PAL algorithm simulations to identify the effect of growth conditions and other parameters on nutritional properties of resulting ingredients.

### Results and Conclusion

The results of this experiment will demonstrate that the algorithm identifies species of yeast, and growth conditions for yeast, that have desired PDCAAS, physiochemical properties, and/or digestibility properties.

### Example 12: Algorithm Performance on Additional Bacteria

### Purpose

The purpose of this experiment is to determine if the method can identify species of bacteria, and growth conditions for the species of bacteria, with desired PDCAAS, physiochemical properties, and/or digestibility properties.

### Methods

The method is used on a series of prokaryotic organisms and matching in vitro data is obtained. Several different growth parameters, such as growth duration, temperature, atmospheric nitrogen, oxygen, and carbon dioxide, are varied as the bacteria are grown. A good correlation of the algorithm with in vitro data will be shown across a large number of bacterial species.

### Results and Conclusion

The results of this experiment will demonstrate that the algorithm identifies species of bacteria, and growth parameters for the bacteria, that have desired PDCAAS, physiochemical properties, and/or digestibility properties.

### Example 13: Algorithm Performance on Organisms Identified in Fermented Foods

### Purpose

The purpose of this experiment was to determine if the method could identify organisms from commonly consumed fermented foods.

### Methods

The algorithm was applied to organisms identified in fermented foods. A sourdough starter was used as an example of a common fermented food. Sourdough starters are highly diverse and contain a complex mixture of organisms (Landis et al, ELife (2021), Vol 10:e61644).

The BioSkryb Resolve™ technology (www.bioskryb.com) was used for flow sorting to sort samples from sourdough starter into high, medium, and low complexity samples. In this technology, flow sorting is used to produce progressively sorted liquid materials, which reduces the complexity of multiple species within each liquid fraction. This enables low complexity samples to be sequenced using modern genome sequencing platforms, such as the Illumina platform, reducing background.

Two samples were provided: “S004” contains a sample that was streaked out on plates and grown first to reduce complexity and “Sourdough” represents the more complex sourdough starter mixture.

Multiple organisms were identified by flow sorting and sequencing, including Lactobacillus species and some species of Archaea. “H” represents the higher complexity sample, “M” is medium sorting, and “Low” represents low complexity or maximum sorting. The organisms were sequencing using BioSkryb's PTA (Primary Template-Directed Amplification) technology which helps provide superior uniformity of DNA amplification (as compared to traditional technique, MDA, Multiple Displacement Amplification). The amplified material was then sequenced using Illumina technology and assembled using typical genome assembly software. Typically, genomes could be assembled to between 73% and 99% completeness.

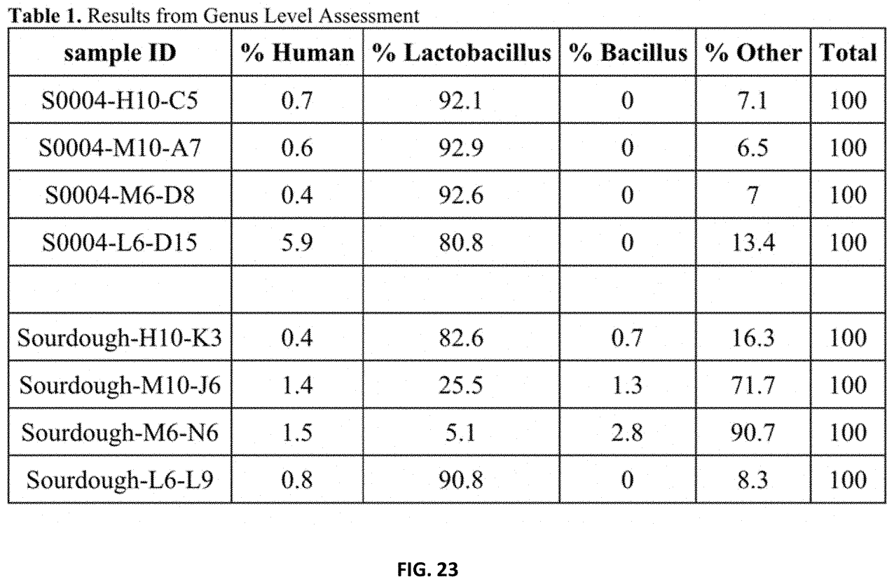

Next, overall taxonomic diversity in the samples using Kraken was assessed (Wood & Salzberg, Genome Biology (2014), Vol 15(R46)). The results of this assessment are shown in FIG. 23. Sample S004 is primarily Lactobacillus, as expected. The Sourdough samples contained in many cases a predominance of Lactobacillus, Bacillus and other species.

To further identify organisms at the species level, identification of the predominant organism in the sourdough starter was determined. The assembly of this organism was identified as Lacticaseibacillus paracasei, a Lactobacillus organism in the family Lactobacillaceae This organism has been identified in sourdough starters (Shen et al, Molecules (2022), Vol 27(11), 3510).

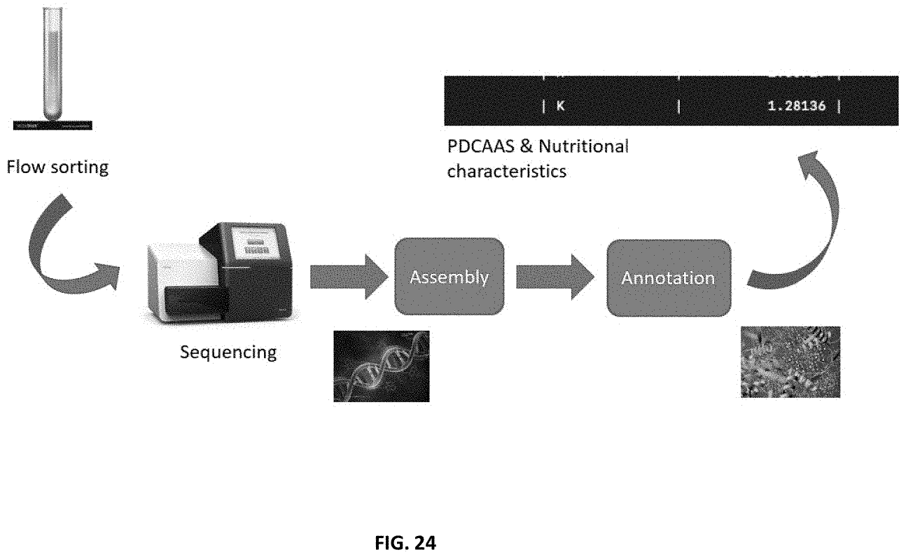

Next, the algorithm as described in Example 1 was applied on the identified organism. FIG. 24 shows an overview of the workflow. A first limiting amino acid of Lysine (K) and an unadjusted PDCAAS score of 1.28 (PDCAAS=1) were determined.

### Results and Conclusion

This example demonstrates that organisms can be identified from fermented foods by sequencing and then analyzed by the nutritional algorithm presented herein to provide estimates of PDCAAS, digestibility, and other characteristics. This workflow could also be used to identify unknown organisms from the environment to apply the algorithm to study their nutritional properties.

### Example 14: Use of Algorithm on a Plant Genome

### Purpose

The purpose of this experiment is to use the method to identify plant species with a desired PDCAAS, physiochemical properties, and/or digestibility properties.

### Methods

To demonstrate that the algorithm can also be used on plant genomes, the Soy genome, which is known to have a PDCAAS of 1, is analyzed (Qin et al, Journal of Agriculture and Food Research (2022), Vol 7, 100265). To calculate the PDCAAS and demonstrate robustness of the algorithm, the Subsetting tool (tool: make_subsets) is used to process a subset of the transcripts and proteins of the soybean genome (Glycine max). A predicted PDCAAS of 1 is obtained from the pipeline, in line with expectations.

### Results and Conclusion

The result of this experiment demonstrates that the algorithm robustly predicts a PDCAAS value for plant species. The method further identifies ideal physiochemical properties, and/or digestibility properties from the plant species.

### Example 15: Use of the Algorithm on Individual Proteins

### Purpose

The purpose of this experiment was to determine if the method can determine an accurate PDCAAS score on individual proteins.

### Methods

The algorithm can also be used to compute scores on a variety of individual proteins (peptide sequences). These can be drawn from any number of public sources such as NCBI or Uniprot, or from the annotation of a novel sequenced genome. One can also use the algorithm to design synthetic sequences with ideal protein properties.

These sequences can be also overexpressed in organisms using a variety of methods. This approach imparts additional protein quality benefit to protein products and foods derived using these organisms. The algorithm was used on several chosen or designed protein sequences and determined the predicted properties to demonstrate function of the algorithm on single peptides.

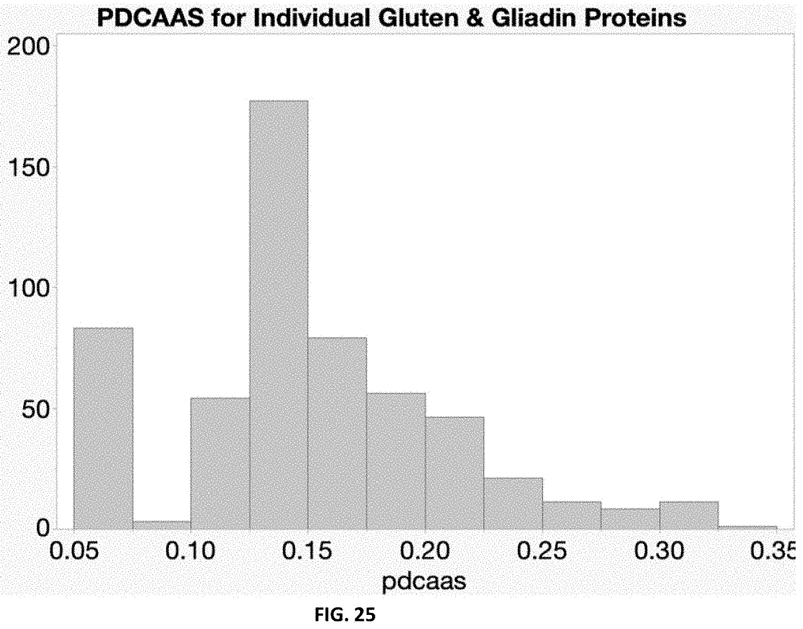

As one example, the algorithm was used on a large number of gluten and gliadin proteins. These proteins are expected to have low PDCAAS. FIG. 25 shows the distribution of PDCAAS scores predicted for these proteins by the algorithm, being low as expected.

Next, the PDCAAS for other individual proteins was analyzed. Bovine Serum Albumin (BSA), expected to be high, gives a predicted PDCAAS of ˜0.84. Casein is expected to be near 1, and the algorithm confirms this, giving Cow Casein a PDCAAS of 0.91. Collagens are expected to be low due to their repetitive nature and are found to be in the 0.4-0.5 range by the algorithm.

### Results and Conclusion

The results of this example showed that the method can be applied to individual proteins or sets of proteins to provide accurate PDCAAS scores. In addition, the method can be used to analyze the nutritional properties of synthetically designed proteins or proteomes.

### Example 16: Use of Algorithm on Proteomics Data

### Purpose

The result of this example is to determine if the method can use proteomics data to identify an organism with a desired PDCAAS.

### Methods

In addition to working from a genome, the algorithm could be run based on proteomics dataset, such as shotgun proteomics. In this case, the abundance values for proteins are given by values provided by the proteomics data, such as pct_nsaf (see below), instead of using the EAP expression adjustment module.

Proteomics, or shotgun proteomics, seeks to identify the proteome complement of whole cells by first fractionating proteins, then separating them with liquid chromatography and subsequently performing analysis with mass spectrometry (Meyer, Methods in Molecular Biology Book Series (2021)). Software then analyzes the mass and charge of peptide fragments to reconstruct the protein sequence and estimate their abundance. One of the most common methods to look at abundance is to analyze the total spectral counts for each peptide/protein and then calculate a percentage of the Normalized Spectral Abundance Factor (NSAF) giving pct_nsaf, which is a measure of the relative abundance of a protein in comparison to the overall protein complement of the sample. Other methods include the spectral index, exponentially modified protein abundance index (emPAI) and distributed normalized spectral abundance factor (dNSAF) (McIlwain et al, BMC BioInformatics (2012), Vol 13(308)).

In the case of proteomics data as an input data set for the algorithm, the EAP module is bypassed for expression adjustment and the protein sequences and abundances (pct_nsaf) are used directly, followed by the amino acid scoring and PDCAAS modules for final estimation of PDCAAS and other properties determined by the algorithm.

### Results and Conclusion

The results of this example demonstrate that the method may be applied to identify organisms with desired PDCAAS using proteomics data.

### Example 17: Production & Characterization of Optimized Protein Product

### Purpose

The purpose of this example is to demonstrate that the method can identify an organism that produces an ideal protein product.

### Methods

Production and characterization of protein products is carried out using organisms recommended by the algorithm. An organism is fermented and protein product is produced, as in Example 7, “Fermentation of protein product & Measurement of amino acid profiles”. One or more final protein products is prepared. The AAP, IVPDCAAS, and IVDIAAS of the protein product is measured. A good correspondence between the algorithm predictions and the test results will be shown. This resulting product will be shown to have a PDCAAS of 1 or greater and DIAAAS>100 with a good balance of essential amino acids, providing excellent nutrition and high protein content.

### Results and Conclusion

The results of this example will demonstrate that the method may identify an organism that produces an ideal protein product with a PDCAAS of 1 or greater, a DIAAAS>100, and a good balance of essential amino acids.

### Example 18: Use of Algorithm to Select an Organism to Produce a Protein Product that Improves the Protein Nutrition Quality of a Food Product

### Purpose

The purpose of this example is to determine if the methods of the disclosure can identify an organism that can produce a protein product that enhances the protein quality of a food product.

### Methods

The algorithm from Example 1 is used to identify and select an organism with high protein nutrition quality, physiochemical properties, and/or digestibility properties, as described in Examples 2, 11, 12, and 14-16. The organism is then fermented as described in Example 7. To achieve a protein product with enhanced protein nutrition quality, physiochemical properties, and/or digestibility properties, the method selects a mix of organisms, as described in Example 10. The resulting protein product can then be used with a variety of flours to increase the overall protein nutrition quality of a subsequent food product. For example, the protein product can be combined with a flour to create a baked product. The PDCAAS and DIAAS of the food product is then determined, revealing a PDCAAS of 1 or greater, a DIAAAS>100, and a good balance of essential amino acids.

### Results and Conclusion

The results of this example will demonstrate that the methods of the disclosure may identify an organism that can produce a protein product that enhances the protein quality of a food product.

### Example 19: Selection and Mixture of Amino Acid Profiles Important for Nutrition, Human Health & Food Ingredient Properties

### Purpose

The purpose of this example is to show that the algorithm presented herein can be used to select an organism, or mixture of organisms, to provide amino acid profiles that are supportive of various diets or health interventions.

### Methods

The sections below discuss various types of amino acid profiles used in different dietary interventions. The algorithms described above for amino acid calculations, along with the Mixture algorithm, can be used to achieve these desired profiles. Organisms that are especially high in any given amino acid can be beneficial, either alone or in combination as a mixture, for Human health. Amino acid scores for specific EAA for multiple organisms are computed by the pipeline. Using the pipeline presented herein, organisms are selected that are exceptionally high or low in various amino acids and recommend them for blending or mixture into food ingredients with customized properties for Human diets.

Alterations in EAA mixture can affect energy homeostasis (Xiao & Guo, Molecular Metabolism (2022) Vol 57:101393). Deprivations of particular amino acids in a diet can lead to a variety of impacts. For example, Isoleucine deprivation is predicted to stimulate fat loss (Du et al, Amino Acids (2012), Vol 43:725-734). By querying the nutritional database (APDB), Yarrowia sp. E02, B02, and C11 are predicted to have low levels of Isoleucine. Thus, selection of microbial organisms can produce food ingredients geared toward weight loss.

### Muscle Health

Amino acids influence the creation, health, and maintenance of muscle (Kamei, Nutrients (2020), Vol 12(1):261). Leucine can improve muscle growth. An examination of the data shows that Lacticaseibacillus paracasei is high in Leucine. Thus, this organism could be selected to improve Leucine content in a food ingredient product designed to support increased muscle.

The algorithm from Example 1 is used to identify and select an organism with high protein nutrition quality, physiochemical properties, digestibility properties, and high leucine, as described in Examples 2, 11, 12, and 14-16. The organism is then fermented as described in Example 7. To achieve a protein product with enhanced protein nutrition quality, physiochemical properties, and/or digestibility properties, the method selects a mix of organisms, as described in Example 10. The resulting protein product can then be used with a variety of flours to increase the overall protein nutrition quality of a subsequent animal food product. The PDCAAS and DIAAS of the food product is then determined, revealing a PDCAAS of 1 or greater, a DIAAAS>100, and high leucine.

### Brain Health

Tryptophan (Trp) (W) is an amino acid associated with treatment of depression and sleep disorders, as well as health of the brain-gut axis (Kaluzna-Czaplinksa, Critical Reviews in Food Science & Nutrition (2019), Vol 59(1):72-88). The algorithm from Example 1 can identify organisms that are high in Tryptophan. Searching the nutritional database (APDB) one organism especially high in Tryptophan is Lipomyces starkeyi. By cross-referencing the nutritional database presented herein, the predicted PDCAAS score of this organism is 0.97. Therefore, this organism could be an excellent source of Tryptophan.

The algorithm from Example 1 is used to identify and select an organism with high protein nutrition quality, physiochemical properties, digestibility properties, and high tryptophan, as described in Examples 2, 11, 12, and 14-16. The organism is then fermented as described in Example 7. To achieve a protein product with enhanced protein nutrition quality, physiochemical properties, and/or digestibility properties, the method selects a mix of organisms, as described in Example 10. The resulting protein product can then be used with a variety of flours to increase the overall protein nutrition quality of a subsequent animal food product. The PDCAAS and DIAAS of the food product is then determined, revealing a PDCAAS of 1 or greater, a DIAAAS>100, and high tryptophan.

### Pregnancy Health

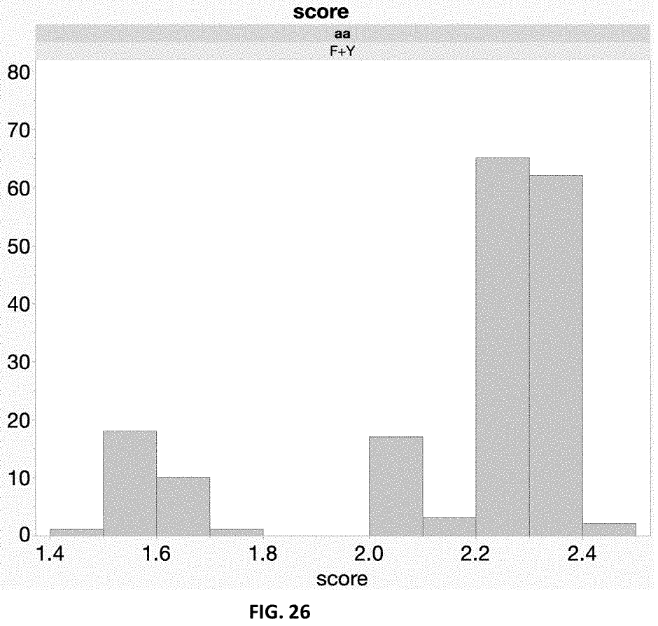

The correct balance of amino acids is critical in nutrition during pregnancy and neonatal development (Hsu & Tain, Nutrients (2020), Vol 12(6):1763). Pregnant women have higher nutritional requirements, and there is a lack of specific amino acid guidelines for pregnancy (Elango & Ball, Advances in Nutrition (2016), Vol 7(4): 839S-844S). It has been reported that Lysine requirements are higher in pregnancy, as well as phenylalanine (Phe) (F). FIG. 26 shows the distribution of F+Y scores in the APDB database, showing a wide distribution of levels of these amino acids in various organisms. One non-limiting example organism with high F+Y score is Wickerhamomyces anomalus NRRL-Y-366-8, which is predicted to have a PDCAAS of 0.96. Thus, blending of appropriate microbial protein ingredients could be used to provide optimal nutrition in pregnancy. In addition, proper amino acid supplementation during pregnancy may help prevent hypertension.

To create a food product, the algorithm from Example 1 is used to identify and select an organism with high protein nutrition quality, physiochemical properties, digestibility properties, and high lysine and/or phenylalanine, as described in Examples 2, 11, 12, and 14-16. The organism is then fermented as described in Example 7. To achieve a protein product with enhanced protein nutrition quality, physiochemical properties, and/or digestibility properties, the method selects a mix of organisms, as described in Example 10. The resulting protein product can then be used with a variety of flours to increase the overall protein nutrition quality of a subsequent animal food product. The PDCAAS and DIAAS of the food product is then determined, revealing a PDCAAS of 1 or greater, a DIAAAS>100, and high lysine and/or phenylalanine.

### Elderly Health

Nutritional supplementation with EAA can improve quality of life in the elderly (Rondanelli et al, Clinical Nutrition (2011), Vol 30(5):571-577). This includes improvement in depressive symptoms, muscle strength, activity, and overall nutrition. Long-term supplementation of EAA for the elderly can help reverse age-related muscle deterioration (sarcopenia). In particular, use of BCAA and Leucine may be important in stimulating muscle development (Fujita & Volpi, The Journal of Nutrition (2006), Vol 136(1): 227S-280S). Providing appropriate food ingredients high in EAA may support elderly health, mood, and muscle maintenance.

The algorithm from Example 1 is used to identify and select an organism with high protein nutrition quality, physiochemical properties, digestibility properties, and high EAA, as described in Examples 2, 11, 12, and 14-16. The organism is then fermented as described in Example 7. To achieve a protein product with enhanced protein nutrition quality, physiochemical properties, and/or digestibility properties, the method selects a mix of organisms, as described in Example 10. The resulting protein product can then be used with a variety of flours to increase the overall protein nutrition quality of a subsequent animal food product. The PDCAAS and DIAAS of the food product is then determined, revealing a PDCAAS of 1 or greater, a DIAAAS>100, and high EAA.

### Epilepsy

The algorithm from Example 1 is used to identify and select an organism with high protein nutrition quality, physiochemical properties, digestibility properties, and high ketogenic amino acids, as described in Examples 2, 11, 12, and 14-16. The organism is then fermented as described in Example 7. To achieve a protein product with enhanced protein nutrition quality, physiochemical properties, and/or digestibility properties, the method selects a mix of organisms, as described in Example 10. The resulting protein product can then be used with a variety of flours to increase the overall protein nutrition quality of a subsequent animal food product. The PDCAAS and DIAAS of the food product is then determined, revealing a PDCAAS of 1 or greater, a DIAAAS>100, and high ketogenic amino acids.

### Diabetes

Patients with diabetes may benefit from diets reduced in BCAA (Dutta & Khandelwal, The Journal of Clinical Endocrinology & Metabolism (2023), Vol 108(2): e27-28).

The algorithm from Example 1 is used to identify and select an organism with high protein nutrition quality, physiochemical properties, digestibility properties, and low BCAA, as described in Examples 2, 11, 12, and 14-16. The organism is then fermented as described in Example 7. To achieve a protein product with enhanced protein nutrition quality, physiochemical properties, and/or digestibility properties, the method selects a mix of organisms, as described in Example 10. The resulting protein product can then be used with a variety of flours to increase the overall protein nutrition quality of a subsequent animal food product. The PDCAAS and DIAAS of the food product is then determined, revealing a PDCAAS of 1 or greater, a DIAAAS>100, and low BCAA.

### Cancer

There is a complex interplay between interventions in cancer, designed to either starve a tumor, or to improve muscle wasting in cancer patients due to treatment. Results of numerous studies of BCAA supplementation in cancer have been controversial (Lee & Blanton, Nutrition & Health (2023), Vol 0(0)). However, it appears that BCAA supplementation for severely malnourished patients or those in whom the cancer has been destroyed may be beneficial.

The algorithm from Example 1 is used to identify and select an organism with high protein nutrition quality, physiochemical properties, digestibility properties, and high BCAA, as described in Examples 2, 11, 12, and 14-16. The organism is then fermented as described in Example 7. To achieve a protein product with enhanced protein nutrition quality, physiochemical properties, and/or digestibility properties, the method selects a mix of organisms, as described in Example 10. The resulting protein product can then be used with a variety of flours to increase the overall protein nutrition quality of a subsequent animal food product. The PDCAAS and DIAAS of the food product is then determined, revealing a PDCAAS of 1 or greater, a DIAAAS>100, and high BCAA.

### Methionine & Cysteine in Plant Protein

Many plant proteins are deficient in Methionine, and Cysteine which is synthesized from Methionine (Whitcomb et al, Frontiers in Plant Science (2020), Vol 11(1118)). A review of the nutritional database presented herein shows organisms high in Methionine & Cysteine including numerous strains of Bacillus subtilis, Yarrowia lipolytica, and Brettanomyces bruxellensis. Attempts to increase methionine content in plants require transgenic organisms and have met with limited success. However, the approach presented herein, using microbial fermentation, enhances methionine and cysteine content of food ingredients naturally.

The algorithm from Example 1 is used to identify and select an organism with high protein nutrition quality, physiochemical properties, digestibility properties, and high methionine and/or cysteine, as described in Examples 2, 11, 12, and 14-16. The organism is then fermented as described in Example 7. To achieve a protein product with enhanced protein nutrition quality, physiochemical properties, and/or digestibility properties, the method selects a mix of organisms, as described in Example 10. The resulting protein product can then be used with a variety of flours to increase the overall protein nutrition quality of a subsequent food product. The PDCAAS and DIAAS of the animal food product is then determined, revealing a PDCAAS of 1 or greater, a DIAAAS>100, and high methionine and/or cysteine.

### Lysine and Histidine

Lysine is typically the most limiting amino acid in cereal foods (Poutanen et al, Nutrition Reviews (2022) Vol 80(6):1648-1663). Fermentation of cereals can be an excellent way to enhance Lysine content. A review of the nutritional database presented herein shows Fructilactobacillus sanfrancicensis and Lactobacillus helveticus as non-limiting examples of microorganisms high in Lysine.

Lysine and Histidine, as well as other amino acids, may experience loss during baking. Thus, selection of microorganisms and choice of appropriate downstream processing techniques may be used to maximize presence of certain amino acids after baking or cooking loss.

The algorithm from Example 1 is used to identify and select an organism with high protein nutrition quality, physiochemical properties, digestibility properties, and high lysine and/or histidine, as described in Examples 2, 11, 12, and 14-16. The organism is then fermented as described in Example 7. To achieve a protein product with enhanced protein nutrition quality, physiochemical properties, and/or digestibility properties, the method selects a mix of organisms, as described in Example 10. The resulting protein product can then be used with a variety of flours to increase the overall protein nutrition quality of a subsequent animal food product. The PDCAAS and DIAAS of the food product is then determined, revealing a PDCAAS of 1 or greater, a DIAAAS>100, and high lysine and/or histidine.

### Results and Conclusion

This example shows that the algorithm presented herein can be used to find organisms used to create ideal mixtures with amino acid profiles suited for a variety of dietary and health interventions.

### Example 20: Use of Algorithm to Select an Organism to Produce a Protein Product that Improves the Protein Nutrition Quality of a Companion Animal Food Product

### Purpose

The purpose of this example is to determine if the methods of the disclosure can identify an organism that can produce a protein product that enhances the protein quality of a companion animal food product.

### Methods

In addition to Human foods, the algorithm presented herein can be used to select organisms suitable for adding protein to companion animal foods. The algorithm from Example 1 is used to identify and select an organism with high protein nutrition quality, physiochemical properties, and/or digestibility properties, as described in Examples 2, 11, 12, and 14-16. The organism is then fermented as described in Example 7. To achieve a protein product with enhanced protein nutrition quality, physiochemical properties, and/or digestibility properties, the method selects a mix of organisms, as described in Example 10. The resulting protein product can then be used with a variety of flours to increase the overall protein nutrition quality of a subsequent companion animal food product. The PDCAAS and DIAAS of the companion animal food product is then determined, revealing a PDCAAS of 1 or greater, a DIAAAS>100, and a good balance of essential amino acids.

### Results and Conclusion

The results of this example demonstrate the methods of the disclosure may identify an organism that can produce a protein product that enhances the protein quality of a companion animal food product.

### Example 21: Use of Algorithm to Select an Organism to Produce a Protein Product that Improves the Protein Nutrition Quality of a Farm Animal Food Product

### Purpose

The purpose of this example is to determine if the methods of the disclosure can identify an organism that can produce a protein product that enhances the protein quality of an animal food product. Such approaches may find application in farm animals or aquaculture. Such approaches are also expected to provide environmental benefits such as reduced use of traditional food sources and reduced greenhouse gas emissions.

### Methods

In addition to Human foods, the algorithm presented herein can be used to select organisms suitable for adding protein to animal foods. The algorithm from Example 1 is used to identify and select an organism with high protein nutrition quality, physiochemical properties, and/or digestibility properties, as described in Examples 2, 11, 12, and 14-16. The organism is then fermented as described in Example 7. To achieve a protein product with enhanced protein nutrition quality, physiochemical properties, and/or digestibility properties, the method selects a mix of organisms, as described in Example 10. The resulting protein product can then be used with a variety of flours to increase the overall protein nutrition quality of a subsequent animal food product. The PDCAAS and DIAAS of the animal food product is then determined, revealing a PDCAAS of 1 or greater, a DIAAAS>100, and a good balance of essential amino acids.

### Results and Conclusion

The results of this example demonstrate the methods of the disclosure may identify an organism that can produce a protein product that enhances the protein quality of a farm animal food product.

### NUMBERED EMBODIMENTS

Notwithstanding the appended claims, the disclosure sets forth the following numbered embodiments:

Embodiment 1. An in silico method for selecting an organism as a source of protein, the method comprising:
a. accessing a genomic library comprising genomic information;
b. creating an adjusted relative abundance proteomic library from the genomic library;
c. creating a functionally-characterized proteomic library from the adjusted relative abundance proteomic library;
d. supplying a computational algorithm with data from the functionally characterized proteomic library;
e. computing a protein nutritional quality score with the computational algorithm; and
f. selecting an organism as a source of protein from the genomic library, wherein the computational algorithm selects the organism based on its computed protein nutritional quality scores being above a desired threshold.

Embodiment 2. The method of embodiment 1, wherein the computational algorithm comprises one or more computational algorithms.

Embodiment 3. The method of embodiments 1 to 2, wherein one of the one or more computational algorithms is a machine learning algorithm.

Embodiment 4. The method of embodiments 1 to 3, wherein the machine learning algorithm further computes protein digestibility factors.

Embodiment 5. The method of embodiments 1 to 4, wherein the protein digestibility factor is an alpha helix/beta-sheet ratio.

Embodiment 6. The method of embodiments 1 to 5, wherein the machine learning algorithm improves the accuracy of computing the digestibility factors.

Embodiment 7. The method of embodiments 1 to 6, wherein the protein nutritional quality score is a protein expression estimation score, a protein molecular weight calculation score, and/or an amino acid analysis score.

Embodiment 8. The method of embodiments 1 to 7, wherein the genomic library comprises a plurality of nucleotide sequences from a single organism.

Embodiment 9. The method of embodiments 1 to 8, wherein the genomic library comprises a plurality of nucleotide sequences from a plurality of organisms.

Embodiment 10. The method of embodiments 1 to 9, wherein the genomic library comprises at least one partial whole genome nucleotide sequence of an organism.

Embodiment 11. The method of embodiments 1 to 10, wherein the genomic library comprises a plurality of partial whole genome nucleotide sequences of a plurality of organisms.

Embodiment 12. The method of embodiments 1 to 11, wherein the genomic library comprises a plurality of complete whole genome nucleotide sequences of a plurality of organisms.

Embodiment 13. The method of embodiments 1 to 12, wherein the genomic library is from a public genomic database.

Embodiment 14. The method of embodiments 1 to 13, wherein the genomic library comprises a genomic sequence from a prokaryote.

Embodiment 15. The method of embodiments 1 to 14, wherein the genomic library comprises a genomic sequence from a eukaryote.

Embodiment 16. The method of embodiments 1 to 15, wherein the genomic library comprises a genomic sequence from an unknown organism.

Embodiment 17. The method of embodiments 1 to 16, wherein the genomic library comprises a genomic sequence obtained from de novo sequencing.

Embodiment 18. The method of embodiments 1 to 17, wherein the genomic library comprises a genomic sequence obtained from isolation sequencing.

Embodiment 19. The method of embodiments 1 to 18, wherein creating the adjusted relative abundance proteomic library comprises direct translation of the genomic library.

Embodiment 20. The method of embodiments 1 to 19, wherein creating the adjusted relative abundance proteomic library comprises direct translation of the microbial genomic library, and subsequent characterization of relative protein abundance.

Embodiment 21. The method of embodiments 1 to 20, wherein creating the adjusted relative abundance proteomic library comprises calculation of a codon adaptation index parameter for each protein in the library.

Embodiment 22. The method of embodiments 1 to 21, wherein creating the adjusted relative abundance proteomic library comprises calculation of a delta factor parameter comprising the Euclidean distance between each protein and the average ribosomal protein for each protein in the library.

Embodiment 23. The method of embodiments 1 to 22, wherein creating the adjusted relative abundance proteomic library comprises mass spectrometry based shotgun proteomics.

Embodiment 24. The method of embodiments 1 to 23, wherein creating the functionally characterized proteomic library comprises calculating one or more functional attributes of the library.

Embodiment 25. The method of embodiments 1 to 24, wherein creating the functionally characterized proteomic library comprises calculating one or more functional attributes selected from the group consisting of: overall amino acid composition, essential amino acid composition, non-essential amino acid composition, most limiting amino acid, and estimated nitrogen content.

Embodiment 26. The method of embodiments 1 to 25, wherein one or more modules of the computational algorithm may utilize a machine learning method selected from the group consisting of linear regression, kernel ridge regression, logistic regression, neural networks, support vector machines, decision trees, hidden Markov models, Bayesian networks, a Gram-Schmidt process, reinforcement-based learning, self-supervised learning, cluster-based learning, hierarchical clustering, language models, bi-directional Long-Short-Term-Memory and genetic algorithms.

Embodiment 27. The method of embodiments 1 to 26, wherein the protein nutritional quality score is a Protein Digestibility Corrected Amino Acid Score (PDCAAS).

Embodiment 28. The method of embodiments 1 to 27, wherein the protein nutritional quality score is a Digestible Indispensable Amino Acid Score (DIAAS).

Embodiment 29. The method of embodiments 1 to 28, wherein the protein nutritional quality score is an in vitro Protein Digestibility Corrected Amino Acid Score (IVPDCAAS).

Embodiment 30. The method of embodiments 1 to 29, wherein the protein nutritional quality score is an in vitro Digestible Indispensable Amino Acid Score (IVDIAAS).

Embodiment 31. The method of embodiments 1 to 30, wherein the desired threshold of the IVDIAAS score is at least 100.

Embodiment 32. The method of embodiments 1 to 31, wherein the desired threshold of the protein nutritional quality score is PDCAAS of at least 0.75.

Embodiment 33. The method of embodiments 1 to 32, wherein the desired threshold of the protein nutritional quality score is DIAAS of at least 75.

Embodiment 34. The method of embodiments 1 to 33, wherein the desired threshold of the protein nutritional quality score is IVPDCAAS of at least 0.75.

Embodiment 35. The method of embodiments 1 to 34, wherein the desired threshold of the protein nutritional quality score is IVDIAAS of at least 75.

Embodiment 36. The method of embodiments 1 to 35, wherein the protein nutritional quality score is a PDCAAS, DIAAS, IVPDCAAS, IVDIAAS, or any combination thereof.

Embodiment 37. The method of embodiments 1 to 36, wherein the desired threshold of the protein nutritional quality score is PDCAAS, IVPDCAAS, DIAAS, IVDIAAS, or any combination thereof, each with a score of at least 0.75 and 75, respectively.

Embodiment 38. The method of embodiments 1 to 37, wherein the protein nutritional quality score is a Euclidean distance metric.

Embodiment 39. The method of embodiments 1 to 38, wherein the desired threshold of the Euclidean distance is less than 0.1 from a target amino acid distribution.

Embodiment 40. The method of embodiments 1 to 39, wherein the target amino acid distribution is 60% essential amino acids and 40% non-essential amino acids.

Embodiment 41. The method of embodiments 1 to 40, wherein the target amino acid distribution is 70% essential amino acids and 30% non-essential amino acids.

Embodiment 42. The method of embodiments 1 to 41, wherein the target amino acid distribution is an amino acid distribution of proteins from milk.

Embodiment 43. The method of embodiments 1 to 42, wherein the target amino acid distribution is an amino acid distribution of proteins from egg.

Embodiment 44. The method of embodiments 1 to 43, wherein the target amino acid distribution is an amino acid distribution of proteins from beef.

Embodiment 45. The method of embodiments 1 to 44, wherein the selected organism comprises a PDCAAS of at least 0.75.

Embodiment 46. The method of embodiments 1 to 45, wherein the selected organism comprises a DIAAS of at least 75.

Embodiment 47. The method of embodiments 1 to 46, wherein the selected organism comprises a Euclidean distance less than 0.1 from a target amino acid distribution.

Embodiment 48. The method of embodiments 1 to 47, wherein the target amino acid distribution is 60% essential amino acids and 40% non-essential amino acids.

Embodiment 49. The method of embodiments 1 to 48, wherein the target amino acid distribution is 70% essential amino acids and 30% non-essential amino acids.

Embodiment 50. The method of embodiments 1 to 49, wherein the target amino acid distribution is an amino acid distribution of proteins from milk.

Embodiment 51. The method of embodiments 1 to 50, wherein the target amino acid distribution is an amino acid distribution of proteins from egg.

Embodiment 52. The method of embodiments 1 to 51, wherein the target amino acid distribution is an amino acid distribution of proteins from beef.

Embodiment 53. The method of embodiments 1 to 52, wherein the selected organism is fermented to produce a protein ingredient.

Embodiment 54. The method of embodiments 1 to 53, wherein the protein ingredient is used to improve the protein nutritional quality of a food product.

Embodiment 55. The method of embodiments 1 to 54, wherein the food product is a human food product.

Embodiment 56. The method of embodiments 1 to 55, wherein the human food product improves muscle health, brain health, pregnancy health, elderly health, epilepsy, diabetes, or cancer.

Embodiment 57. The method of embodiments 1 to 56, wherein the food product is a companion animal food product.

Embodiment 58. The method of embodiments 1 to 57, wherein the food product is a farm animal food product.

Embodiment 59. An in silico method for determining an organism's protein nutritional quality from a genomic library, comprising:
a. accessing a genomic library;
b. creating an adjusted relative abundance proteomic library from the genomic library;
c. creating a functionally-characterized proteomic library from the adjusted relative abundance proteomic library; and
d. supplying a computational algorithm with data from the functionally characterized proteomic library, wherein the computational algorithm computes a protein nutritional quality score for an organism from the genomic library.

Embodiment 60. The method of embodiment 59, wherein the computation algorithm is a machine learning algorithm.

Embodiment 61. The method of embodiments 59 to 60, wherein the machine learning algorithm further computes protein digestibility factors.

Embodiment 62. The method of embodiments 59 to 61, wherein the protein digestibility factor is an alpha helix/beta-sheet ratio.

Embodiment 63. The method of embodiments 59 to 62, wherein the machine learning algorithm improves the accuracy of computing the digestibility factors.

Embodiment 64. The method of embodiments 59 to 63, wherein the organism protein nutritional quality score is a protein expression estimation score, a protein molecular weight calculation score, and/or an amino acid analysis score.

Embodiment 65. The method of embodiments 59 to 64, wherein the genomic library comprises a plurality of nucleotide sequences from a single microorganism.

Embodiment 66. The method of embodiments 59 to 65, wherein the genomic library comprises a plurality of nucleotide sequences from a plurality of organisms.

Embodiment 67. The method of embodiments 59 to 66, wherein the genomic library comprises at least one partial whole genome nucleotide sequence of an organism.

Embodiment 68. The method of embodiments 1 to 67, wherein the genomic library comprises a plurality of partial whole genome nucleotide sequences of a plurality of organisms.

Embodiment 69. The method of embodiments 1 to 68, wherein the genomic library comprises at least one complete whole genome nucleotide sequence of an organism.

Embodiment 70. The method of embodiments 1 to 69, wherein the genomic library comprises a plurality of complete whole genome nucleotide sequences of a plurality of organisms.

Embodiment 71. The method of embodiments 1 to 70, wherein the genomic library is from a public genomic database.

Embodiment 72. The method of embodiments 1 to 71, wherein the genomic library comprises a genomic sequence from a prokaryote.

Embodiment 73. The method of embodiments 1 to 72, wherein the genomic library comprises a genomic sequence from a eukaryote.

Embodiment 74. The method of embodiments 1 to 73, wherein the eukaryote is a higher plant.

Embodiment 75. The method of embodiments 1 to 74, wherein the genomic library comprises a genomic sequence from an unknown organism.

Embodiment 76. The method of embodiments 1 to 75, wherein the genomic library comprises a genomic sequence obtained from de novo sequencing.

Embodiment 77. The method of embodiments 1 to 76, wherein the genomic library comprises a genomic sequence obtained from isolation sequencing.

Embodiment 78. The method of embodiments 1 to 77, wherein creating the adjusted relative abundance proteomic library comprises direct translation of the genomic library.

Embodiment 79. The method of embodiments 1 to 78, wherein creating the adjusted relative abundance proteomic library comprises direct translation of the genomic library, and subsequent characterization of relative protein abundance.

Embodiment 80. The method of embodiments 1 to 79, wherein creating the adjusted relative abundance proteomic library comprises calculation of a codon adaptation index parameter for each protein in the library.

Embodiment 81. The method of embodiments 1 to 80, wherein creating the adjusted relative abundance proteomic library comprises calculation of a delta factor parameter comprising the Euclidean distance between each protein and the average ribosomal protein for each protein in the library.

Embodiment 82. The method of embodiments 1 to 81, wherein creating the adjusted relative abundance proteomic library comprises mass spectrometry-based shotgun proteomics.

Embodiment 83. The method of embodiments 1 to 82, wherein creating the functionally characterized proteomic library comprises calculating one or more functional attributes of the library.

Embodiment 84. The method of embodiments 1 to 83, wherein creating the functionally characterized proteomic library comprises calculating one or more functional attributes selected from the group consisting of: overall amino acid composition, essential amino acid composition, non-essential amino acid composition, most limiting amino acid, and estimated nitrogen content.

Embodiment 85. The method of embodiments 1 to 84, wherein one or more modules of the computational algorithm may utilize a machine learning method selected from the group consisting of linear regression, kernel ridge regression, logistic regression, neural networks, support vector machines, decision trees, hidden Markov models, Bayesian networks, a Gram-Schmidt process, reinforcement-based learning, self-supervised learning, cluster-based learning, hierarchical clustering, language models, bi-directional Long-Short-Term-Memory and genetic algorithms.

Embodiment 86. The method of embodiments 1 to 85, wherein the organism protein nutritional quality score is a Protein Digestibility Corrected Amino Acid Score (PDCAAS).

Embodiment 87. The method of embodiments 1 to 86, wherein the organism protein nutritional quality score is a Digestible Indispensable Amino Acid Score (DIAAS).

Embodiment 88. The method of embodiments 1 to 87, wherein the organism protein nutritional quality score is an in vitro Protein Digestibility Corrected Amino Acid Score (IVPDCAAS).

Embodiment 89. The method of embodiments 1 to 88, wherein the organism protein nutritional quality score is an in vitro Digestible Indispensable Amino Acid Score (IVDIAAS).

Embodiment 90. The method of embodiments 1 to 89, wherein the IVDIAAS score is at least 100.

Embodiment 91. The method of embodiments 1 to 90, wherein the organism protein nutritional quality score is PDCAAS of at least 0.75.

Embodiment 92. The method of embodiments 1 to 91, wherein the organism protein nutritional quality score is DIAAS of at least 75.

Embodiment 93. The method of embodiments 1 to 92, wherein the organism protein nutritional quality score is IVPDCAAS of at least 0.75.

Embodiment 94. The method of embodiments 1 to 93, wherein the organism protein nutritional quality score is IVDIAAS of at least 75.

Embodiment 95. The method of embodiments 1 to 94, wherein the organism protein nutritional quality score is a PDCAAS, DIAAS, IVPDCAAS, IVDIAAS, or any combination thereof.

Embodiment 96. The method of embodiments 1 to 95, wherein the organism protein nutritional quality score is PDCAAS, IVPDCAAS, DIAAS, IVDIAAS, or any combination thereof, each with a score of at least 0.75 and 75, respectively.

Embodiment 97. The method of embodiments 1 to 96, wherein the organism protein nutritional quality score is a Euclidean distance metric.

Embodiment 98. The method of embodiments 1 to 97, wherein the Euclidean distance is less than 0.1 from a target amino acid distribution.

Embodiment 99. The method of embodiments 1 to 98, wherein the target amino acid distribution is 60% essential amino acids and 40% non-essential amino acids.

Embodiment 100. The method of embodiments 1 to 99, wherein the target amino acid distribution is 70% essential amino acids and 30% non-essential amino acids.

Embodiment 101. The method of embodiments 1 to 100, wherein the target amino acid distribution is an amino acid distribution of proteins from milk

Embodiment 102. The method of embodiments 1 to 101, wherein the target amino acid distribution is an amino acid distribution of proteins from egg.

Embodiment 103. The method of embodiments 1 to 102, wherein the target amino acid distribution is an amino acid distribution of proteins from beef.

Embodiment 104. A processor-readable non-transitory medium storing code representing instructions to be executed by a processor, the code comprising code to cause the processor to:
a. access a microbial genomic library;
b. create an adjusted relative abundance microbial proteomic library from the microbial genomic library;
c. create a functionally characterized microbial proteomic library from the adjusted relative abundance microbial proteomic library;
d. supply a computational algorithm with data from the functionally characterized microbial proteomic library,
wherein the computational algorithm computes a protein nutritional quality score for a microorganism from the microbial genomic library.

Embodiment 105. An in silico method for determining an organism's protein nutritional quality from a genomic library, comprising:
a. accessing a genomic library;
b. creating an adjusted relative abundance proteomic library from the genomic library;
c. creating a functionally characterized proteomic library from the adjusted relative abundance proteomic library; and
d. supplying a machine learning model with data from the functionally characterized proteomic library, wherein the machine learning model computes a protein nutritional quality score for an organism from the genomic library.

Embodiment 106. The method of embodiment 105, wherein the organism is a prokaryote, and the genomic library is a prokaryotic genomic library.

Embodiment 107. The method of embodiments 105 to 106, wherein the organism is a eukaryote, and the genomic library is a eukaryotic genomic library.

Embodiment 108. The method of embodiments 105 to 107, wherein the organism is a yeast, and the genomic library is a yeast genomic library.

Embodiment 109. The method of embodiments 105 to 108, wherein the organism is a plant, and the genomic library is a plant genomic library.

Embodiment 110. A processor-readable non-transitory medium storing code representing instructions to be executed by a processor, the code comprising code to cause the processor to:
a. access a genomic library;
b. create an adjusted relative abundance proteomic library from the genomic library;
c. create a functionally characterized proteomic library from the adjusted relative abundance proteomic library;
d. supply a machine learning model with data from the functionally characterized proteomic library; and
e. determine, utilizing the machine learning model, a protein nutritional quality score for an organism from the genomic library.

Embodiment 111. The processor-readable non-transitory medium of embodiment 110, wherein the organism is a prokaryote, and the genomic library is a prokaryotic genomic library.

Embodiment 112. The processor-readable non-transitory medium of embodiments 110 to 111, wherein the organism is a eukaryote, and the genomic library is a eukaryotic genomic library.

Embodiment 113. The processor-readable non-transitory medium of embodiments 110 to 112, wherein the organism is a yeast, and the genomic library is a yeast genomic library.

Embodiment 114. The processor-readable non-transitory medium of embodiments 110 to 113, wherein the organism is a plant, and the genomic library is a plant genomic library.

Embodiment 115. An in silico method for determining an organism's protein nutritional quality from a proteomic library, comprising:
a. accessing a proteomic library;
b. optionally creating an adjusted relative abundance proteomic library from the proteomic library;
c. creating a functionally characterized proteomic library from the adjusted relative abundance proteomic library; and
d. supplying a computational algorithm with data from the functionally characterized proteomic library, wherein the computational algorithm computes a protein nutritional quality score for an organism from the proteomic library.

Embodiment 116. The method of embodiment 115, wherein the organism is a prokaryote, and the proteomic library is a prokaryotic proteomic library.

Embodiment 117. The method of embodiments 115 to 116, wherein the organism is a eukaryote, and the proteomic library is a eukaryotic proteomic library.

Embodiment 118. The method of embodiments 115 to 117, wherein the organism is a yeast, and the proteomic library is a yeast proteomic library.

Embodiment 119. The method of embodiments 115 to 118, wherein the organism is a plant, and the proteomic library is a plant proteomic library.

Embodiment 120. The method of embodiments 115 to 119, wherein the proteomic library comprises one or more protein amino acid sequences.

Embodiment 121. A processor-readable non-transitory medium storing code representing instructions to be executed by a processor, the code comprising code to cause the processor to:
a. access a proteomic library;
b. create an adjusted relative abundance proteomic library from the proteomic library;
c. create a functionally characterized proteomic library from the adjusted relative abundance proteomic library;
d. supply a computational algorithm with data from the functionally characterized proteomic library,
wherein the computational algorithm computes a protein nutritional quality score for an organism from the proteomic library.

Embodiment 122. The processor-readable non-transitory medium of embodiment 121, wherein the organism is a prokaryote, and the proteomic library is a prokaryotic proteomic library.

Embodiment 123. The processor-readable non-transitory medium of embodiments 121 to 122, wherein the organism is a eukaryote, and the proteomic library is a eukaryotic proteomic library

Embodiment 124. The processor-readable non-transitory medium of embodiments 121 to 123, wherein the organism is a yeast, and the proteomic library is a yeast proteomic library.

Embodiment 125. The processor-readable non-transitory medium of embodiments 121 to 124, wherein the organism is a plant, and the proteomic library is a plant proteomic library.

Embodiment 126. The processor-readable non-transitory medium of embodiments 121 to 125, wherein the proteomic library comprises one or more protein amino acid sequences.

Embodiment 127. An in silico method for determining a microbial organism's protein nutritional quality from a microbial genomic library, comprising:
a. accessing a microbial genomic library;
b. creating an adjusted relative abundance microbial proteomic library from the microbial genomic library;
c. creating a functionally-characterized microbial proteomic library from the adjusted relative abundance microbial proteomic library; and
d. supplying a machine learning model with data from the functionally characterized microbial proteomic library,
wherein the machine learning model computes a protein nutritional quality score for a microorganism from the microbial genomic library; and,
wherein the method uses a mixture prediction algorithm to increase the average protein nutritional quality score of a composition by mixing one composition with a lower protein nutritional quality score with one or more compositions to improve the amino acid balance.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not, be taken as an acknowledgement or any form of suggestion that they constitute valid prior art or form part of the common general knowledge in any country in the world.

### PATENT CITATIONS

1. U.S. Pat. No. 9,700,071B2, “Nutritive fragments, proteins, and methods”
2. U.S. 63/428,014, “Methods of using protein ingredients to make doughs, baked products, soups, beverages and snacks”
3. U.S. 63/419,237, “Carrier and filler food compositions with high protein and low lipid content”

### NON-PATENT CITATIONS

1. Altschul, Gish, Miller, Myers & Lipman, “Basic Local Alignment Search Tool”, Journal of Molecular Biology (1990), Vol 215(3):403-410
2. Bai, Qin, Sun & Long, “Relationship Between Molecular Structure Characteristics of Feed Proteins and Protein in vitro Digestibility and Solubility”, Amino Acids, August 2016, Vol 29(8):1159-1165
3. Barnett & Kim, “Protein Instability”, Food Storage Stability (1997), Ch. 3:75-87
4. Batbayar, “Optimized Solid State and Submerged Fermentation of Pea Protein Enriched Flour to Compare the Effects on Protein Quality and Functional Properties”, Thesis, University of Saskatchewan, January 2022
5. Cantarel, Korf, Robb, Parra, Ross, Moore, Holt, Alvarado & Yandell, “MAKER: an Easy-to-use Annotation Pipeline Designed for Emerging Model Organism Genomes”, Genome Research (2008), Vol 18:188-196
6. Carbonaro, Maselli & Nucara, “Relationship between Digestibility and Secondary Structure of Raw and Thermally treated Legume Proteins: a Fourier Transform Infrared (FT-IR) Spectroscopic Study”, August 2012, Vol 43:911-921
7. Chandra, Tunnerman & Lofstedt, “Transformer-based Deep Learning for Predicting Protein Properties in the Life Sciences”, ELife (2023), Vol 12:e82819
8. Chima, Mathews, Morgan, Johnson & Buiten, “Physicochemical Characterization of Interactions between Blueberry Polyphenols and Food Proteins from Dairy and Plant sources”, Foods, September 2022, Vol 11, 2846
9. Drozdetskiy, Cole, Procter & Barton, “JPred4, a Protein Secondary Structure Prediction Server”, Nucleic Acids Research, July 2015, Vol 43(W1): W389-394
10. Du, Meng, Zhang & Guo, “Isoleucine or Valine Deprivation Stimulates Fat Loss via Increasing Energy Expenditure and Regulating Lipid Metabolism in WAT”, Amino Acids (2012), Vol 43:725-734
11. Dutta & Khandelwal, “Lifestyle Interventions Reduce the Risk of Type 2 Diabetes Through Decreasing Branched-Chain Amino Acids: Newer Insights”, The Journal of Clinical Endocrinology & Metabolism (2023), Vol 108(2): e27-28
12. Edwards & Cummings, “The Protein Quality of Mycoprotein”, Proceedings of the Nutrition Society, May 2010, Vol. 69
13. Elango & Ball, “Protein and Amino acid Requirements during Pregnancy”, Advances in Nutrition (2016), Vol 7(4): 839S-844S
14. Elizalde, Pilosof & Bartholomai, “Prediction of Emulsion Instability from Emulsion Composition and Physiochemical Properties of Proteins”, Journal of Food Science, January 1991, Vol 56(1):116-120
15. Farsi, Gallegos, Koutsidis, Nelson, Finnigan, Cheung, Munoz-Munoz & Commane, “Substituting Meat for Mycoprotein reduces Genotoxicity and Increases the Abundance of Beneficial Microbes in the Gut: Mycomeat, a Randomized Crossover Control Trail”, European Journal of Nutrition (2023), doi.org/10.1007/s00394-023-03088-x
16. Ferreira, Ventorim, Almeida, Silveira & Silveira, “Protein Abundance Prediction through Machine Learning Methods”, Journal of Molecular Biology, November 2021, Vol 433(22), 167267
17. Ferruz, Schmidt & Hocker, “ProtGPT2 is a Deep Unsupervised Language Model for Protein Design”, Nature Communications (2022), Vol 13(4348)
18. Fujita & Volpi, “Amino Acids and Muscle Loss with Aging”, The Journal of Nutrition (2006), Vol 136(1): 227S-280S
19. Griffith & Holehouse, “PARROT is a Flexible Recurrent Neural Network Framework for Analysis of Large Protein Datasets”, ELife, September 2021, Vol 10:e70576
20. Grossman & McClements, “Current Insights into Protein Solubility: A Review of its Importance for Alternative Proteins”, Food Hydrocolloids, April 2023, Vol 137, 108416
21. Gu, Bk, Wu, Lu, Nawaz, Barrow, Dunshea & Suleria, “Impact of Processing and Storage on Protein Digestibility and Bioavailability of Legumes”, Food Reviews International (2022)
22. Guruprasad, Reddy & Pandit, “Correlation between Stability of a Protein and its Dipeptide Composition: a Novel Approach for Predicting in vivo Stability of a Protein from its Primary Sequence”, Protein Engineering Design & Selection, December 1990, Vol 4(2):155-161
23. Gu, Bk, Wu, Lu, Nawaz, Barrow, Dunshea & Suleria, “Impact of Processing and Storage on Protein Digestibility and Bioavailability of Legumes”, Food Reviews International (2022)
24. Hoffman & Falvo, “Protein—Which is best?”, Journal of Sports Science & Medicine, September 2004, Vol 3(3):118-130
25. Hsu & Tain, “Amino Acids and Developmental Origins of Hypertension”, Nutrients (2020), Vol 12(6):1763
26. Humpenoder, Bodirsky, Weindl, Lotze-Campen, Linder & Popp, “Projected Environmental Benefits of Replacing Beef with Microbial Protein”, Nature, May 2022, Vol 605:90-96
27. Ishihama, Schmidt, Rappsilber, Mann, Hartl, Kerner & Frishman, “Protein Abundance Profiling of the E. coli Cytosol”, BMC Genomics, February 2007, Vol 9(102):1-17
28. Ismail, Senarante-Lenagala, Stube & Brackenridge, “Protein Demand: Review of Plant and Animal Proteins used in Alternative Protein Product Development and Production”, Animal Frontiers, October 2020, Vol 10(4):53-63
29. Ismi, Pulungan & Afiahayati, “Deep Learning for Protein Secondary Structure Prediction: pre and post-AlphaFold”, Computational and Structural Biotechnology Journal (2022), Vol 20:6271-6286
30. Jach, Serefko, Ziaja & Kieliszek, “Yeast Protein as an Easily Accessible Food Source”, Metabolites, January 2022, Vol 12(1):63
31. Jahn, Rekdal & Sommer, “Microbial Foods for Improving Human and Planetary Health”, Cell (2023), Vol 183(3):469-478
32. Janczyk, Franke & Souffrant, “Nutritional Value of Chlorella vulgaris: Effects of Ultrasonication and Electroporation on Digestibility in Rats”, Animal Feed Science & Technology, January 2007, Vol 132(1-2):163-169
33. Jeyakumar & Lawrence, “Microbial Fermentation for Reduction of Antinutritional Factors”, Current Developments in Biotechnology & BioEngineering (2022), Ch 10:239-260.
34. Jones, Karpol, Friedman, Maru & Tracy, “Recent Advances in Single Cell Protein use as a Feed Ingredient in Aquaculture”, Current Opinion in Biotechnology (2020), Vol 61:189-197
35. Kaluzna-Czaplinksa, Gatarek, Chirumbolo, Chartrand & Bjorklund, “How Important is Tryptophan in Human health?”, Critical Reviews in Food Science & Nutrition (2019), Vol 59(1):72-88
36. Kamei, Hatazawa, Uchitomi, Yoshimura & Miura, “Regulation of Skeletal Muscle Function by Amino Acids”, Nutrients (2020), Vol 12(1):261
37. Karlin, Mrazek, Campbell & Kaiser, “Characterizations of Highly Expressed Genes of Four Fast-growing Bacteria”, J. Bacteriology, September 2001, Vol 183(17)
38. Kim & Kwon, AttSec: Protein Secondary Structure Prediction by Capturing Local Patterns from Attention Map, Research Square (2023), Preprint
39. Koren, Walenz, Berlin, Miller, Bergman & Phillippy, “Canu: Scalable and Accurate Long-read Assembly via Adaptive k-mer Weighting and Repeat Separation”, Genome Research (2017), Vol 27:722-736
40. Kuforiji & Aboaba, “The use of Candida Tropicalis as a Source of Single Cell Protein”, International Journal of Biomedical and Health Sciences (2009), Vol 5(1):7-14
41. Landis, Oliverio, McKenney, Nichols, Kfoury, Biango-Daniels, Shell, Madden, Shapiro, Sakunala, Drake, Robbat, Booker, Dunn, Fierer & Wolfe, “The diversity and function of sourdough starter microbiomes”, eLife, January 2021, Vol 10:e61644
42. Lee & Blanton, “The Effect of Branched-chain Amino Acid Supplementation on Cancer Treatment”, Nutrition & Health (2023), Vol 0(0).
43. Li, Liu, Zou, He, Xu, Zhou & Li, “In vitro Protein Digestibility of Pork Products is Affected by the Method of Processing”, Food Research International, February 2017, Vol 92:88-94
44. Lin, Yang, Chi & Ma, “Effect of Protein Types on Structure and Digestibility of Starch-Protein-Lipids Complexes”, LWT, December 2020, Vol 134
45. Lobry & Gautier, “Hydrophobicity, Expressivity, and Aromaticity are the Major Trends of Amino-Acid Usage in 999 Escherichia coli Chromosome-encoded Genes”, Nucleic Acids Research, August 1994, Vol 22:3174-3180
46. Madani, Krause, Greene, et al, “Large Language Models Generate Functional Protein Sequences Across Diverse Families”, Nature Biotechnology (2023)
47. Malvar, Bhagavathula, Balaguer, Sharma & Chandra, “Machine Learning can Guide Experimental Approaches for Protein Digestibility Estimations”, Springer Nature (2021), arXiv:2211.00625
48. Mateo & Stein, “Apparent and Standardized Ileal Digestibility of Amino Acids in Yeast Extract and Spray Dried Plasma Protein by Weanling Pigs”, Canadian Journal of Animal Science, May 2007, Vol 87(3):381-383
49. McIlwain, Mathews, Bereman, Rubel, MacCoss & Noble, “Estimating Relative Abundances of Proteins from Shotgun Proteomics Data”, BMC BioInformatics, November 2012, Vol 13(308)
50. Meyer, “Qualitative and Quantitative Shotgun Proteomics Data Analysis from Data-Dependent Acquisition Mass Spectrometry”, Shotgun Proteomics, Methods in Molecular Biology Book Series, March 2021
51. Moura, Savageau & Alves, “Relative Amino Acid Signatures of Organisms and Environments”, Plos One, October, 2013, Vol 8(10): e77319
52. Nakai, “Structure-function Relationships of Food Proteins: with an Emphasis on the Importance of Protein Hydrophobicity”, Journal of Agricultural and Food Chemistry, July 1983, Vol 31(4):676-683
53. Poutanen, Karlund, Gomez-Gallego, Johannson, Scheers, Marklinder, Eriksen, Silventoinen, Nordlund, Sozer, Hahnineva, Kolehmainen & Landberg, “Grains—a Major Source of Sustainable Protein for Health”, Nutrition Reviews (2022), Vol 80(6):1648-1663
54. Qin, Wang & Luo, “A Review on Plant-based Proteins from Soybean: Health Benefits and Soy Product Development”, Journal of Agriculture and Food Research, March 2022, Vol 7, 100265
55. Ritala, Hakkinen, Toivari & Wiebe, “Single Cell Protein—State of the art, Industrial landscape, and patents 2001-2016”, Frontiers in Microbiology, October 2017, Vol 8
56. Rondanelli, Opizzi, Antoniello, Boschi, ladarola, Pasini, Aquilani & Dioguardi, “Effect of Essential Amino Acid Supplementation on Quality of Life, Amino Acid Profile and Strength in Institutionalized Elderly Patients”, Clinical Nutrition (2011), Vol 30(5):571-577
57. Salazar-Lopez, Barco-Mendoza, Zuniga-Martinez, Dominguez-Avila, Robles-Sanchez, Ochoa & Gonzalez-Aguilar, “Single-Cell Protein Production as a Strategy to Reincorporate Food Waste and Agro By-Products Back into the Processing Chain”, BioEngineering (2022), Vol 9(11):623
58. Samtiya, Aluko & Dhewa, “Plant Food Anti-Nutritional Factors and their Reduction Strategies: an Overview”, Food Production, Processing & Nutrition (2020), Vol 2(6)
59. Schaafsma, “The Protein-Digestibility Corrected Amino Acid Score (PDCAAS)—a Concept for Describing Protein Quality in Foods and Food Ingredients: A Critical Review”, Journal of AOAC International, May 2005, Vol 88(3):988-994
60. Sharp & Li, “The Codon Adaptation Index—a Measure of Directional Synonymous Codon Usage Bias, and its Potential Applications”, Nucleic Acids Research, 1987, Vol 15:1281-1295
61. Shen, Shi, Dong, Yang, Lu, Lu & Wang, “Screening of Sourdough Starter Strains and Improvements in the Quality of Whole Wheat Steamed Bread”, Molecules, May 2022, Vol 27(11), 3510
62. Sheth & Patel, “Production, Economics, and Marketing of Yeast Single Cell Protein”, Food Microbiology Based Entrepreneurship, January 2023, pp 133-152
63. Shreck, Zeltwanger, Bailey, Jennings, Meyer & Cole, “Effects of Protein Supplementation to Steers Consuming Low-quality Forages on Greenhouse Gas Emissions”, Journal of Animal Science (2021), Vol 99(7): skab147
64. Seeman, “Prokka: Rapid Prokaryotic Genome Annotation”, BioInformatics (2014), Vol 30(14):2068-2069
65. Tartaglia, Cavalli, Pellarin & Caflisch, “The role of Aromaticity, Exposed Surface, and Dipole Moment in Determining Protein Aggregation Rates”, Protein Science, July 2004, Vol 13(7):1939-1941
66. Tavano, Neves & Junior, “In vitro versus in vivo Protein Digestibility Techniques for Calculating PDCAAS (Protein-Digestibility Corrected Amino Acid Score) Applied to Chickpea Fractions”, Food Research International, November 2016, Vol 89:756-763
67. Townsend & Nakai, “Relationships between Hydrophobicity and Foaming Characteristics of Food Proteins”, Journal of Food Science, March 1983, Vol 48(2):588-594
68. Vaser & Sikic, “Time- and Memory-efficient Genome Assembly with Raven”, Nature Computational Science (2021), Vol 1:332-336
69. Wang, Tibbetts, Berrue, McGinn, MacQuarrie, Puttaswamy, Patelakis, Schmidt, Melanson, Mackenzie, “A Rat Study to Evaluate the Protein Quality of Three Green Microalgal Species and the Impact of Mechanical Cell Wall Disruption”, Foods, November 2020, Vol 9(11):1531
70. Wang, Prince & Marcotte, “Mass Spectrometry of the M. smegmatis Proteome: Protein Expression Levels Correlate with Function, Operons, and Codon bias”, Genome Research, May 2005, Vol 15(8):1118-1126
71. Whitcomb, Rakpenthai, Bruckner, Fischer, Parmar, Erban, Kopka, Hawkesford & Hoefgen, “Cysteine and Methionine Biosynthetic Enzymes have Distinct Effects on Seed Nutritional Quality and on Molecular Phenotypes Associated with Accumulation of a Methionine-Rich Seed Storage Protein in Rice”, Frontiers in Plant Science (2020), Vol 11(1118)
72. Whon, Ahn, Yang, Kim, Kim, Kim, Hong, Jung, Choi, Lee & Roh, “ODFM, an Omics Data Resource from Microorganisms Associated with Fermented Foods”, Scientific Data, April 2021, Vol 8(113):1-10
73. Wolfe, Rutherfurd, Kim & Moughan, “Protein Quality as Determined by the Digestible Indispensable Amino Acid Score: Evaluation of Factors Underlying the Calculation”, Nutrition Reviews, September 2016, Vol 74(9):584-99
74. Wood & Salzberg, “Kraken: Ultrafast Metagenomic Sequence Classification using Exact Alignments”, Genome Biology, March 2014, Vol 15(R46)
75. Xiao & Guo, “Impacts of Essential Amino Acids on Energy Balance”, Molecular Metabolism (2022), Vol 57:101393
76. Yu, “Protein Secondary Structures (a-helix and b-sheet) at a Cellular level and Protein Fractions in Relation to Rumen Degradation Behaviors of Protein: a New Approach”, British Journal of Nutrition, 2005, Vol 94:655-665
77. Yuan, He, Zhang & Ma, “Ultrasound-assisted Enzymatic Hydrolysis of Yeast Beta-glucan Catalyzed by Beta-glucanase: Chemical and Microstructural Analysis”, Ultrasonics Sonochemistry (2022), Vol 86:106012
78. Zeng, Chen, Zhang, Li, Wang & Sun, “Nutritional Value and Physicochemical Characteristics of Alternative Protein for Meat and Dairy—A Review”, Foods, October 2022, Vol 11(21):3326
79. Zhao, Zhu, Qin, Pan, Sun, Bao, Hasham & Farouk, “Physicochemical Properties of Dietary Protein as Predictors for Digestibility or Releasing Percentage of Amino Acids in Monogastrics under in-vitro Conditions”, Italian Journal of Animal Science, March 2022, Vol 21(1):507-521

What is claimed is:

1. An in silico method for selecting an organism as a source of protein, the method comprising:
a. accessing a genomic library comprising genomic information;
b. creating an adjusted relative abundance proteomic library from the genomic library;
c. creating a functionally-characterized proteomic library from the adjusted relative abundance proteomic library;
d. supplying a computational algorithm with data from the functionally characterized proteomic library;
e. computing a protein nutritional quality score with the computational algorithm; and
f. selecting an organism as a source of protein from the genomic library, wherein the computational algorithm selects the organism based on its computed protein nutritional quality scores being above a desired threshold.

2. The method of claim 1, wherein the computational algorithm comprises one or more computational algorithms.

3. The method of claim 2, wherein one of the one or more computational algorithms is a machine learning algorithm.

4. The method of claim 3, wherein the machine learning algorithm further computes protein digestibility factors.

5. The method of claim 4, wherein the protein digestibility factor is an alpha helix/beta-sheet ratio.

6. The method of claim 3, wherein the machine learning algorithm improves the accuracy of computing the digestibility factors.

7. The method of claim 1, wherein the protein nutritional quality score is a protein expression estimation score, a protein molecular weight calculation score, and/or an amino acid analysis score.

8. The method of claim 1, wherein the genomic library comprises a plurality of nucleotide sequences from a single organism.

9. The method of claim 1, wherein the genomic library comprises a plurality of nucleotide sequences from a plurality of organisms.

10. The method of claim 1, wherein the genomic library comprises at least one partial whole genome nucleotide sequence of an organism.

11. The method of claim 1, wherein the genomic library comprises a plurality of partial whole genome nucleotide sequences of a plurality of organisms.

12. The method of claim 1, wherein the genomic library comprises a plurality of complete whole genome nucleotide sequences of a plurality of organisms.

13. The method of claim 1, wherein the genomic library is from a public genomic database.

14. The method of claim 1, wherein the genomic library comprises a genomic sequence from a prokaryote.

15. The method of claim 1, wherein the genomic library comprises a genomic sequence from a eukaryote.

16. The method of claim 1, wherein the genomic library comprises a genomic sequence from an unknown organism.

17. The method of claim 1, wherein the genomic library comprises a genomic sequence obtained from de novo sequencing.

18. The method of claim 1, wherein the genomic library comprises a genomic sequence obtained from isolation sequencing.

19. The method of claim 1, wherein creating the adjusted relative abundance proteomic library comprises direct translation of the genomic library.

20. The method of claim 1, wherein creating the adjusted relative abundance proteomic library comprises direct translation of the microbial genomic library, and subsequent characterization of relative protein abundance.

21. The method of claim 1, wherein creating the adjusted relative abundance proteomic library comprises calculation of a codon adaptation index parameter for each protein in the library.

22. The method of claim 1, wherein creating the adjusted relative abundance proteomic library comprises calculation of a delta factor parameter comprising the Euclidean distance between each protein and the average ribosomal protein for each protein in the library.

23. The method of claim 1, wherein creating the adjusted relative abundance proteomic library comprises mass spectrometry based shotgun proteomics.

24. The method of claim 1, wherein creating the functionally characterized proteomic library comprises calculating one or more functional attributes of the library.

25. The method of claim 1, wherein creating the functionally characterized proteomic library comprises calculating one or more functional attributes selected from the group consisting of: overall amino acid composition, essential amino acid composition, non-essential amino acid composition, most limiting amino acid, and estimated nitrogen content.

26. The method of claim 1, wherein one or more modules of the computational algorithm may utilize a machine learning method selected from the group consisting of linear regression, kernel ridge regression, logistic regression, neural networks, support vector machines, decision trees, hidden Markov models, Bayesian networks, a Gram-Schmidt process, reinforcement-based learning, self-supervised learning, cluster-based learning, hierarchical clustering, language models, bi-directional Long-Short-Term-Memory and genetic algorithms.

27. The method of claim 1, wherein the protein nutritional quality score is a Protein Digestibility Corrected Amino Acid Score (PDCAAS).

28. The method of claim 1, wherein the protein nutritional quality score is a Digestible Indispensable Amino Acid Score (DIAAS).

29. The method of claim 1 wherein the protein nutritional quality score is an in vitro Protein Digestibility Corrected Amino Acid Score (IVPDCAAS).

30. The method of claim 1, wherein the protein nutritional quality score is an in vitro Digestible Indispensable Amino Acid Score (IVDIAAS).

31. The method of claim 30, wherein the desired threshold of the IVDIAAS score is at least 100.

32. The method of claim 1, wherein the desired threshold of the protein nutritional quality score is PDCAAS of at least 0.75.

33. The method of claim 1, wherein the desired threshold of the protein nutritional quality score is DIAAS of at least 75.

34. The method of claim 1, wherein the desired threshold of the protein nutritional quality score is IVPDCAAS of at least 0.75.

35. The method of claim 1, wherein the desired threshold of the protein nutritional quality score is IVDIAAS of at least 75.

36. The method of claim 1, wherein the protein nutritional quality score is a PDCAAS, DIAAS, IVPDCAAS, IVDIAAS, or any combination thereof.

37. The method of claim 1, wherein the desired threshold of the protein nutritional quality score is PDCAAS, IVPDCAAS, DIAAS, IVDIAAS, or any combination thereof, each with a score of at least 0.75 and 75, respectively.

38. The method of claim 1, wherein the protein nutritional quality score is a Euclidean distance metric.

39. The method of claim 38, wherein the desired threshold of the Euclidean distance is less than 0.1 from a target amino acid distribution.

40. The method of claim 39, wherein the target amino acid distribution is 60% essential amino acids and 40% non-essential amino acids.

41. The method of claim 39, wherein the target amino acid distribution is 70% essential amino acids and 30% non-essential amino acids.

42. The method of claim 39, wherein the target amino acid distribution is an amino acid distribution of proteins from milk.

43. The method of claim 39, wherein the target amino acid distribution is an amino acid distribution of proteins from egg.

44. The method of claim 39, wherein the target amino acid distribution is an amino acid distribution of proteins from beef.

45. The method of claim 32, wherein the selected organism comprises a PDCAAS of at least 0.75.

46. The method of claim 33, wherein the selected organism comprises a DIAAS of at least 75.

47. The method of claim 38, wherein the selected organism comprises a Euclidean distance less than 0.1 from a target amino acid distribution.

48. The method of claim 47, wherein the target amino acid distribution is 60% essential amino acids and 40% non-essential amino acids.

49. The method of claim 47, wherein the target amino acid distribution is 70% essential amino acids and 30% non-essential amino acids.

50. The method of claim 47, wherein the target amino acid distribution is an amino acid distribution of proteins from milk.

51. The method of claim 47, wherein the target amino acid distribution is an amino acid distribution of proteins from egg.

52. The method of claim 47, wherein the target amino acid distribution is an amino acid distribution of proteins from beef.

53. The method of claim 1, wherein the selected organism is fermented to produce a protein ingredient.

54. The method of claim 53, wherein the protein ingredient is used to improve the protein nutritional quality of a food product.

55. The method of claim 54, wherein the food product is a human food product.

56. The method of claim 55, wherein the human food product improves muscle health, brain health, pregnancy health, elderly health, epilepsy, diabetes, or cancer.

57. The method of claim 54, wherein the food product is a companion animal food product.

58. The method of claim 54, wherein the food product is a farm animal food product.

59. An in silico method for determining an organism's protein nutritional quality from a genomic library, comprising:
a. accessing a genomic library;
b. creating an adjusted relative abundance proteomic library from the genomic library;
c. creating a functionally-characterized proteomic library from the adjusted relative abundance proteomic library; and
d. supplying a computational algorithm with data from the functionally characterized proteomic library,
wherein the computational algorithm computes a protein nutritional quality score for an organism from the genomic library.

60. The method of claim 59, wherein the computation algorithm is a machine learning algorithm.

61. The method of claim 60, wherein the machine learning algorithm further computes protein digestibility factors.

62. The method of claim 61, wherein the protein digestibility factor is an alpha helix/beta-sheet ratio.

63. The method of claim 61, wherein the machine learning algorithm improves the accuracy of computing the digestibility factors.

64. The method of claim 59, wherein the organism protein nutritional quality score is a protein expression estimation score, a protein molecular weight calculation score, and/or an amino acid analysis score.

65. The method of claim 59, wherein the genomic library comprises a plurality of nucleotide sequences from a single microorganism.

66. The method of claim 59, wherein the genomic library comprises a plurality of nucleotide sequences from a plurality of organisms.

67. The method of claim 59, wherein the genomic library comprises at least one partial whole genome nucleotide sequence of an organism.

68. The method of claim 59, wherein the genomic library comprises a plurality of partial whole genome nucleotide sequences of a plurality of organisms.

69. The method of claim 59, wherein the genomic library comprises at least one complete whole genome nucleotide sequence of an organism.

70. The method of claim 59, wherein the genomic library comprises a plurality of complete whole genome nucleotide sequences of a plurality of organisms.

71. The method of claim 59, wherein the genomic library is from a public genomic database.

72. The method of claim 59, wherein the genomic library comprises a genomic sequence from a prokaryote.

73. The method of claim 59, wherein the genomic library comprises a genomic sequence from a eukaryote.

74. The method of claim 73, wherein the eukaryote is a higher plant.

75. The method of claim 59, wherein the genomic library comprises a genomic sequence from an unknown organism.

76. The method of claim 59, wherein the genomic library comprises a genomic sequence obtained from de novo sequencing.

77. The method of claim 59, wherein the genomic library comprises a genomic sequence obtained from isolation sequencing.

78. The method of claim 59, wherein creating the adjusted relative abundance proteomic library comprises direct translation of the genomic library.

79. The method of claim 59, wherein creating the adjusted relative abundance proteomic library comprises direct translation of the genomic library, and subsequent characterization of relative protein abundance.

80. The method of claim 59, wherein creating the adjusted relative abundance proteomic library comprises calculation of a codon adaptation index parameter for each protein in the library.

81. The method of claim 59, wherein creating the adjusted relative abundance proteomic library comprises calculation of a delta factor parameter comprising the Euclidean distance between each protein and the average ribosomal protein for each protein in the library.

82. The method of claim 59, wherein creating the adjusted relative abundance proteomic library comprises mass spectrometry-based shotgun proteomics.

83. The method of claim 59, wherein creating the functionally characterized proteomic library comprises calculating one or more functional attributes of the library.

84. The method of claim 59, wherein creating the functionally characterized proteomic library comprises calculating one or more functional attributes selected from the group consisting of: overall amino acid composition, essential amino acid composition, non-essential amino acid composition, most limiting amino acid, and estimated nitrogen content.

85. The method of claim 59, wherein one or more modules of the computational algorithm may utilize a machine learning method selected from the group consisting of linear regression, kernel ridge regression, logistic regression, neural networks, support vector machines, decision trees, hidden Markov models, Bayesian networks, a Gram-Schmidt process, reinforcement-based learning, self-supervised learning, cluster-based learning, hierarchical clustering, language models, bi-directional Long-Short-Term-Memory and genetic algorithms.

86. The method of claim 59, wherein the organism protein nutritional quality score is a Protein Digestibility Corrected Amino Acid Score (PDCAAS).

87. The method of claim 59, wherein the organism protein nutritional quality score is a Digestible Indispensable Amino Acid Score (DIAAS).

88. The method of claim 59, wherein the organism protein nutritional quality score is an in vitro Protein Digestibility Corrected Amino Acid Score (IVPDCAAS).

89. The method of claim 59, wherein the organism protein nutritional quality score is an in vitro Digestible Indispensable Amino Acid Score (IVDIAAS).

90. The method of claim 89, wherein the IVDIAAS score is at least 100.

91. The method of claim 59, wherein the organism protein nutritional quality score is PDCAAS of at least 0.75.

92. The method of claim 59, wherein the organism protein nutritional quality score is DIAAS of at least 75.

93. The method of claim 59, wherein the organism protein nutritional quality score is IVPDCAAS of at least 0.75.

94. The method of claim 59, wherein the organism protein nutritional quality score is IVDIAAS of at least 75.

95. The method of claim 59, wherein the organism protein nutritional quality score is a PDCAAS, DIAAS, IVPDCAAS, IVDIAAS, or any combination thereof.

96. The method of claim 59, wherein the organism protein nutritional quality score is PDCAAS, IVPDCAAS, DIAAS, IVDIAAS, or any combination thereof, each with a score of at least 0.75 and 75, respectively.

97. The method of claim 59, wherein the organism protein nutritional quality score is a Euclidean distance metric.

98. The method of claim 94, wherein the Euclidean distance is less than 0.1 from a target amino acid distribution.

99. The method of claim 98, wherein the target amino acid distribution is 60% essential amino acids and 40% non-essential amino acids.

100. The method of claim 98, wherein the target amino acid distribution is 70% essential amino acids and 30% non-essential amino acids.

101. The method of claim 98, wherein the target amino acid distribution is an amino acid distribution of proteins from milk

102. The method of claim 98, wherein the target amino acid distribution is an amino acid distribution of proteins from egg.

103. The method of claim 98, wherein the target amino acid distribution is an amino acid distribution of proteins from beef.

104. A processor-readable non-transitory medium storing code representing instructions to be executed by a processor, the code comprising code to cause the processor to:
a. access a microbial genomic library;
b. create an adjusted relative abundance microbial proteomic library from the microbial genomic library;
c. create a functionally characterized microbial proteomic library from the adjusted relative abundance microbial proteomic library; and
d. supply a computational algorithm with data from the functionally characterized microbial proteomic library,
wherein the computational algorithm computes a protein nutritional quality score for a microorganism from the microbial genomic library.

105. An in silico method for determining an organism's protein nutritional quality from a genomic library, comprising:
a. accessing a genomic library;
b. creating an adjusted relative abundance proteomic library from the genomic library;
c. creating a functionally characterized proteomic library from the adjusted relative abundance proteomic library; and
d. supplying a machine learning model with data from the functionally characterized proteomic library,
wherein the machine learning model computes a protein nutritional quality score for an organism from the genomic library.

106. The method of claim 105, wherein the organism is a prokaryote, and the genomic library is a prokaryotic genomic library.

107. The method of claim 105, wherein the organism is a eukaryote, and the genomic library is a eukaryotic genomic library.

108. The method of claim 105, wherein the organism is a yeast, and the genomic library is a yeast genomic library.

109. The method of claim 105, wherein the organism is a plant, and the genomic library is a plant genomic library.

110. A processor-readable non-transitory medium storing code representing instructions to be executed by a processor, the code comprising code to cause the processor to:
a. access a genomic library;
b. create an adjusted relative abundance proteomic library from the genomic library;
c. create a functionally characterized proteomic library from the adjusted relative abundance proteomic library;
d. supply a machine learning model with data from the functionally characterized proteomic library; and
e. determine, utilizing the machine learning model, a protein nutritional quality score for an organism from the genomic library.

111. The processor-readable non-transitory medium of claim 110, wherein the organism is a prokaryote, and the genomic library is a prokaryotic genomic library.

112. The processor-readable non-transitory medium of claim 110, wherein the organism is a eukaryote, and the genomic library is a eukaryotic genomic library.

113. The processor-readable non-transitory medium of claim 110, wherein the organism is a yeast, and the genomic library is a yeast genomic library.

114. The processor-readable non-transitory medium of claim 110, wherein the organism is a plant, and the genomic library is a plant genomic library.

115. An in silico method for determining an organism's protein nutritional quality from a proteomic library, comprising:
a. accessing a proteomic library;
b. optionally creating an adjusted relative abundance proteomic library from the proteomic library;
c. creating a functionally characterized proteomic library from the adjusted relative abundance proteomic library; and
d. supplying a computational algorithm with data from the functionally characterized proteomic library,
wherein the computational algorithm computes a protein nutritional quality score for an organism from the proteomic library.

116. The method of claim 115, wherein the organism is a prokaryote, and the proteomic library is a prokaryotic proteomic library.

117. The method of claim 115, wherein the organism is a eukaryote, and the proteomic library is a eukaryotic proteomic library.

118. The method of claim 115, wherein the organism is a yeast, and the proteomic library is a yeast proteomic library.

119. The method of claim 115, wherein the organism is a plant, and the proteomic library is a plant proteomic library.

120. The method of claim 115, wherein the proteomic library comprises one or more protein amino acid sequences.

121. A processor-readable non-transitory medium storing code representing instructions to be executed by a processor, the code comprising code to cause the processor to:
a. access a proteomic library;
b. create an adjusted relative abundance proteomic library from the proteomic library;
c. create a functionally characterized proteomic library from the adjusted relative abundance proteomic library;
d. supply a computational algorithm with data from the functionally characterized proteomic library,
wherein the computational algorithm computes a protein nutritional quality score for an organism from the proteomic library.

122. The processor-readable non-transitory medium of claim 121, wherein the organism is a prokaryote, and the proteomic library is a prokaryotic proteomic library.

123. The processor-readable non-transitory medium of claim 121, wherein the organism is a eukaryote, and the proteomic library is a eukaryotic proteomic library.

124. The processor-readable non-transitory medium of claim 121, wherein the organism is a yeast, and the proteomic library is a yeast proteomic library.

125. The processor-readable non-transitory medium of claim 121, wherein the organism is a plant, and the proteomic library is a plant proteomic library.

126. The processor-readable non-transitory medium of claim 121, wherein the proteomic library comprises one or more protein amino acid sequences.

127. An in silico method for determining a microbial organism's protein nutritional quality from a microbial genomic library, comprising:
a. accessing a microbial genomic library;
b. creating an adjusted relative abundance microbial proteomic library from the microbial genomic library;
c. creating a functionally-characterized microbial proteomic library from the adjusted relative abundance microbial proteomic library; and
d. supplying a machine learning model with data from the functionally characterized microbial proteomic library,
wherein the machine learning model computes a protein nutritional quality score for a microorganism from the microbial genomic library; and,
wherein the method uses a mixture prediction algorithm to increase the average protein nutritional quality score of a composition by mixing one composition with a lower protein nutritional quality score with one or more compositions to improve the amino acid balance.
